(19) United States
(12) Patent Application Publication
(10) Pub. No.: US 2026/0027243 A1
(43) Pub. Date: Jan. 29, 2026

(54) AMPHIPHILIC COMPOUNDS FOR ATTENUATING NEUROTOXICITY OF AMYLOID-beta OLIGOMERS AND DIAGNOSTIC METHODS

(71) Applicant: THE BOARD OF TRUSTEES OF THE UNIVERSITY OF ILLINOIS, Urbana, IL (US)

(72) Inventors: Liviu M. MIRICA, Champaign, IL (US); Zhengxin YU, Champaign, IL (US)

(73) Assignee: THE BOARD OF TRUSTEES OF THE UNIVERSITY OF ILLINOIS, Urbana, IL (US)

(21) Appl. No.: 18/994,092

(22) PCT Filed: Jul. 10, 2023
(86) PCT No.: PCT/US2023/069870

§ 371 Date: Jan. 14, 2025

Related U.S. Application Data
(60) Provisional application No. 63/389,270, filed on Jul. 14, 2022.

(51) Int. Cl. A61K 51/04; C07D 255/02; C07D 405/10; C07D 405/14; C07D 409/14

(52) U.S. Cl. CPC A61K 51/0482; C07D 255/02; C07D 405/10; C07D 405/14; C07D 409/14

(57) ABSTRACT

Herein is disclosed amphiphilic small molecules with different hydrophobic and hydrophilic fragments that show high M binding affinity to both Aβ plaques and oligomers, and selectively binding Aβ oligomers. These amphiphilic compounds can also label the Aβ species in the brain sections of transgenic AD mice, as shown by immunostaining with an Aβ antibody. Certain amphiphilic compounds were found to alleviate the Cu²⁺-Aβ induced toxicity in cell viability assays. Additionally, two compounds, ZY-15-MT and ZY-15-OMe, were found to disrupt the interactions between Aβ oligomers and human neuroblastoma SH-SY5Y cell membranes. These studies show compounds with amphiphilic properties that target Aβ oligomers and modulate the Aβ oligomer-cell membrane interactions can be effective as small molecule AD therapeutics. Also, the disclosed amphiphilic dicyanomethylene compounds that can emit in the near-infrared region and chelate copper can be used as early diagnostic agents for AD.

### RELATED APPLICATIONS

This application claims priority under 35 U.S.C. § 119(e) (or is related to) to U.S. Provisional Patent Application No. 63/389,270, filed Jul. 14, 2022, which is incorporated herein by reference.

### BACKGROUND OF THE INVENTION

The aggregation of misfolded proteins is commonly observed in different neurodegenerative diseases, including the amyloid-β (Aβ) peptides and tau proteins in Alzheimer's disease (Aβ), or α-synuclein in Parkinson's disease. AD is the most prevalent neurodegenerative disease and affects more than 44 million people worldwide, yet there is still a lack of effective treatments. Although the insoluble amyloid plaques have been considered the main hallmark of AD over the past century, recently the soluble Aβ oligomers were found to be the most neurotoxic species that directly affect synapse loss and neuronal injury. Since Aβ oligomer species appear in the early stages of the disease and are the cause of continuous synaptic damage, they have become attractive targets for AD drug development. Therefore, numerous strategies have been developed to target soluble Aβ oligomers by using various antibodies, polypeptides, natural products, as well as small molecules. Small molecules utilized as therapeutic agents for central nervous system (CNS) disorders have advantages over biologics, including low molecular weight for higher blood-brain barrier (BBB) permeability and simpler structures for easy access at low cost. However, due to the lack of a molecular-level understanding of Aβ oligomers' structures, there is no effective way to design small molecules with high and selective Aβ oligomers binding affinity and to modulate their neurotoxicity.

Also, to prevent the severe loss of cognition, the development of early diagnostic agents for AD can lead to a dramatic improvement of patients' life. Currently, there are three U.S. Food and Drug Administration-approved amyloid PET imaging agents, ¹⁸F-flutemetamol, ¹⁸F-florbetaben, and ¹⁸F-florbetapir, yet these imaging agents cannot be used for early diagnosis of AD. More specifically, these agents can only target Aβ plaques rather than Aβ oligomers, which are the dominant species in the early stages of AD. Moreover, the ¹⁸F radioisotope only has a relatively short half-life time of about 110 min and lengthy synthetic procedures are needed for the covalent incorporation of ¹⁸F in the ¹⁸F-labeled PET imaging tracers. Therefore, the clinical usage of these ¹⁸F-labeled PET tracers is limited to large medical centers.

Accordingly, there is a need to develop compounds with high affinity and selectivity toward Aβ oligomers for both therapeutic and diagnostic applications in the early stages of AD.

### SUMMARY

The misfolded proteins or polypeptides commonly observed in neurodegenerative diseases, including Alzheimer's disease (Aβ), are promising drug targets for developing therapeutic agents. To target the amyloid-β (Aβ) peptide plaques and oligomers, the hallmarks of AD, we have developed amphiphilic small molecules with different hydrophobic and hydrophilic fragments. In vitro fluorescence binding assays demonstrate that these amphiphilic compounds show high binding affinity to both Aβ plaques and oligomers, and six of them exhibit selective binding toward Aβ oligomers. These amphiphilic compounds can also label the Aβ species in the brain sections of transgenic AD mice, as shown by immunostaining with an Aβ antibody. Molecular docking studies were performed to obtain structure-affinity relationships. We have found amphiphilic compounds that can alleviate the Cu²⁺-Aβ induced toxicity in cell viability assays. In addition, confocal fluorescence imaging studies provide evidence that two compounds, ZY-15-MT and ZY-15-OMe, can disrupt the interactions between Aβ oligomers and human neuroblastoma SH-SY5Y cell membranes. Overall, these studies strongly suggest that developing compounds with amphiphilic properties that target Aβ oligomers and modulate the Aβ oligomer-cell membrane interactions can be an effective strategy for the development of small molecule AD therapeutics.

This disclosure also presents a series of novel dicyanoethane-4H-pyran (DCM) derivatives and demonstrates their potential applications as both 64Cu positron emission tomography (PET) and Near-Infrared (NIR) imaging agents for Alzheimer's Disease (Aβ).

Accordingly, this disclosure provides an apparatus for a compound of Formula I:

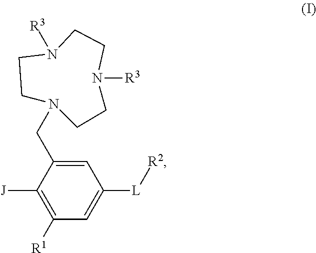

or a salt, metal complex, or combination thereof;
wherein
J is OH, SH, or N(R³)₂;
L is —(CH═CH)ₙ— wherein n is 1, 2, or 0, and L is a direct bond between its two points of attachment when n is 0;
R¹ is H, —OR, or —CH₂N(CH₂CH₂NR³CH₂)₂;
R² is a substituted heterocycle or substituted phenyl; and each R³ is independently —(C₁-C₆)alkyl or H;

In some embodiments, the compounds disclosed herein is a metal complex wherein the metal of the metal complex is copper, a copper ion, or a radioisotope thereof.

This disclosure also provides a method for positron emission tomography (PET) or near infrared (NIR) imaging amyloid-beta oligomers comprising:
a) administering to a subject a composition comprising a compound or metal complex disclosed herein;
b) imaging the brain of the subject for the presence or absence of amyloid-beta oligomers by PET or NIR;
wherein the compound or metal complex selectively binds to the amyloid-beta oligomers when present, thereby providing an early diagnosis for Alzheimer's disease.

Additionally, this disclosure provides a method for reducing the neurotoxicity of amyloid-beta oligomers comprising administering to a subject suffering from an early onset of Alzheimer's disease an effective amount of a composition comprising a compound disclosed herein, wherein the compound selectively binds to amyloid-beta oligomers present in the brain of the subject and reduces the neurotoxicity of amyloid-beta oligomers.

The invention provides novel compounds of Formulas I-V, intermediates for the synthesis of compounds of Formulas I-V, as well as methods of preparing compounds of Formulas I-V. The invention also provides compounds of Formulas I-V that are useful as intermediates for the synthesis of other useful compounds. The invention provides for the use of compounds of Formulas I-V for the manufacture of medicaments useful for the treatment of early stages of Alzheimer's disease in a mammal, such as a human.

The invention provides for the use of the compositions described herein for use in medical diagnostics and medical therapy. The medical therapy can treat the early stages of Alzheimer's disease. The invention also provides for the use of a composition as described herein for the manufacture of a medicament to treat a disease in a mammal, for example, early stages of Alzheimer's disease in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Fluorescence turn-on effects with Aβ₄₂ oligomers and fibrils. (a) ZY-5-OMe, (b) ZY-5-MT (c) ZY-5-DT. circles: compound only; squares: compound +Aβ₄₂ oligomers; triangles: compound +Aβ₄₂ fibrils; [compound]=5 μM; [Aβ₄₂ oligomers]=25 μM; [Aβ₄₂ fibrils]=25 μM.

FIG. 2A-F. Binding constant measurements of ZY-5-OMe (a, b), ZY-5-MT (c, d), ZY-5-DT (e, f) with Aβ₄₂ fibrils (a, c, e), and oligomers (b, d, f).

FIG. 3A-C. Fluorescence microscopy images of 5×FAD mice brain sections co-incubated with amphiphilic compounds (a-c, top), Congo Red (a-c, middle) and merged images (a-c, bottom, along with the Parson's correlation coefficients R). Concentrations: [amphiphilic compounds]=5 μM, [Congo Red]=2.5 μM; scale bar: 125 μm.

FIG. 4A-C. Fluorescence microscopy images of 5×FAD mice brain sections co-incubated with amphiphilic compounds (a-c, top), HJ3.4 (a-c, middle) and merged images (a-c, bottom, along with the Pearson's correlation coefficients R). Concentrations: [amphiphilic compounds]=5 μM, [CF594-HJ 3.4]: 1 μg/mL; scale bar: 125 μm.

FIG. 5. Toxicity of the amphiphilic compounds at different concentrations in mouse neuroblastoma Neuro2A (N2a) cells.

FIG. 6. Cell viability results upon incubation of Neuro2A cells with monomeric Aβ₄₂ in the presence or absence of metal ions and amphiphilic compounds. Concentrations: [Aβ₄₂]=40 μM, [Cu²⁺]=40 μM, [ZY-12-MT]=[ZY-15-OMe]=[ZY-15-MT]=[ZY-17-MT]=[ZY-5-OMe]=[ZY-5-MT]=[ZY-5-DT]=10 μM, [ZY-12-OMe]=5 μM. The error bars represent the standard deviation from five independent experiments, and the statistical analysis was evaluated according to one-way ANOVA (*p<0.05).

FIG. 7A-C. a, b) SH-SY5Y cells were treated with 5 μM Aβ oligomers in the presence or absence of 5 μM ZY-15-MT or 5 μM ZY-15-OMe for 24 h hours before imaging. Red and blue fluorescence indicate the Aβ oligomers and nuclei, respectively. Scar bar, 20 m. c) Three independent experiments were subjected for the statistical analysis and analyzed by one-way ANOVA (*P<0.05, ***P<0.001).

FIG. 8A-F. a, b) SH-SY5Y cells were treated with 5 μM Aβ monomers in the presence or absence of 5 μM Cu²⁺, 5 μM (Cu²⁺+ZY-15-MT) or 5 μM (Cu²⁺+ZY-15-OMe) for 48 h hours before imaging. Red and blue fluorescence indicate the Aβ species and nuclei, respectively. Scar bar, 20 μm. c) Three independent experiments were subjected for the statistical analysis and analyzed by one-way ANOVA (*P<0.05, **P<0.01). d, e) SH-SY5Y cells were treated with 5 μM fibrils in the presence or absence of 5 μM ZY-15-MT or 5 μM ZY-15-OMe for 24 h hours before imaging. Red and blue fluorescence indicate the fibrils and nuclei, respectively. Scar bar, 20 μm. f) Three independent experiments were subjected for the statistical analysis and analyzed by one-way ANOVA.

FIG. 9A-F. Fluorescence turn-on effect of DCM compounds with Aβ₄₂ fibrils and oligomers. [compounds]=5 μM, [Aβ₄₂ fibrils]=25 μM, [Aβ₄₂ oligomers]=2 μM.

FIG. 10. Fluorescence microscopy images of 5×FAD mice brain sections incubated with DCM-OH-1, DCM-OH-1-MT, and DCM-OH-1-DT (left panels), CF594-HJ3.4 (middle panels), and merged images (right panels). Concentrations: [compound]=2 μM [CF594-HJ 3.4]=0.5 μg/mL (Scale bar: 125 μm).

FIG. 11. Fluorescence microscopy images of 5×FAD mice brain sections incubated with DCM-OH-2, DCM-OH-2-MT, and DCM-OH-2-DT (left panels), Thioflavin S (ThS) (middle panels), and merged images (right panels). Concentrations: [compound]=1 μM [ThS]=2. μM (Scale bar: 125 μm).

FIG. 12. Cytotoxicity of DCM compounds at various concentrations in human neuroblastoma cells (SH-SY5Y).

FIG. 13. In vivo accumulation of DCM-OH-2-DT. Fluorescence microscopy images of brain sections from 12-month-old 5×FAD mice administered with DCM-OH-2-DT for 10 days. The brain sections were stained with Thioflavin S (ThS); [ThS]=5 μM, scale bar=125 μm.

FIG. 14A-E. Binding constants of DCM compounds with Aβ₄₂ fibrils.

FIG. 15A-E. Binding constants of DCM compounds with Aβ₄₂ oligomers.

FIG. 16. Data showing Total Radiant Efficiency versus Time for DCM-OH-2-DT.

### DETAILED DESCRIPTION

With the aim of developing therapeutic agents to alleviate the neurotoxicity of Aβ oligomers, we report herein an effective strategy for amphiphilic compounds with different amphiphilicity linking various hydrophobic stilbene derivatives to the hydrophilic triazamacrocycle (Me₂TACN, Chart 1).

The binding affinity of the amphiphilic compounds was measured by in vitro fluorescence saturation assays. These compounds show a high binding affinity to both Aβ fibrils and oligomers in the low micromolar to high nanomolar range. More importantly, some compounds exhibit selective binding affinity toward Aβ oligomers. Molecular docking studies were performed to extract structure-affinity relationships needed for the design of second-generation compounds. Immunostaining assays with an Aβ antibody HJ 3.4 confirms that our compounds are able to label ex vivo Aβ species in the brain sections of transgenic AD mice. Cell toxicity studies demonstrate that certain compounds could rescue mouse neuroblastoma N2a cells from Cu²⁺-Aβ induced toxicity. Finally, the ZY-15-MT and ZY-15-OMe compounds were found to decrease the interaction between Aβ oligomers and human neuroblastoma SH-SY5Y cells as shown via confocal microscopy studies. Overall, these findings provide lead compounds for future optimization and demonstrate an effective strategy for small molecule development.

Chart 1. Molecular structures of the twelve amphiphilic compounds.

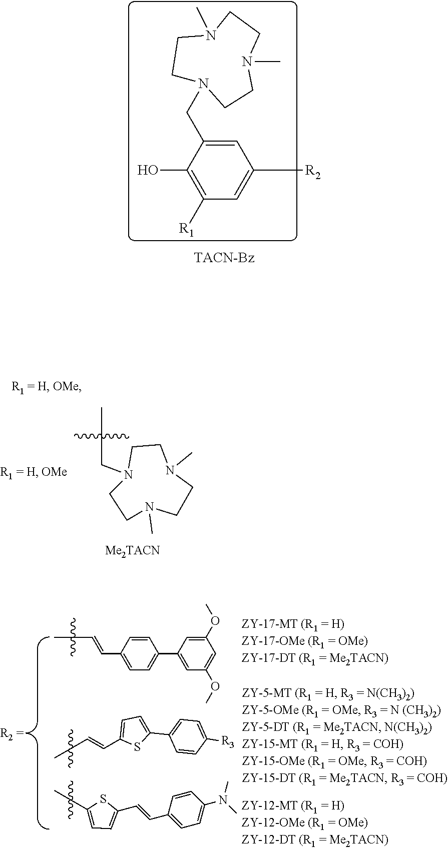

Additional information and data supporting the invention can be found in the following publication by the inventors: Chem. Sci., 2022, 13, 12818 and its Supporting Information; and also, the pre-publication by the inventors DOI 10.26434/chemrxiv-2022-3gxlh-v2, which are incorporated herein by reference in their entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, +10%, +20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

An “effective amount” can also refer to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at a carbon atom or two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents RX where RX is at the ortho-, meta-, orpara-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF3, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as 0, it is bonded to the atom it is substituting by a double bond; for example, a carbon atom substituted with 0 forms a carbonyl group, C═O.

The term “IC₅₀” is generally defined as the concentration required to inhibit a specific biological or biochemical function by half, or to kill 50% of the cells in a designated time period, typically 24 hours.

### Embodiments of the Technology

This disclosure provides a compound of Formula I:

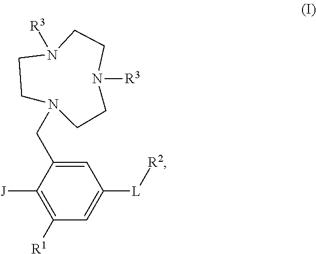

or a salt, metal complex, or combination thereof;
wherein
J is OH, SH, or N(R³)₂;
L is —(CH═CH)ₙ— wherein n is 1, 2, or 0, and L is a direct bond between its two points of attachment when n is 0;
R¹ is H, —OR³, or —CH₂N(CH₂CH₂NR³CH₂)₂;
R² is a substituted heterocycle or substituted phenyl; and each R³ is independently —(C₁-C₆)alkyl or H;

In some embodiments, R² is:

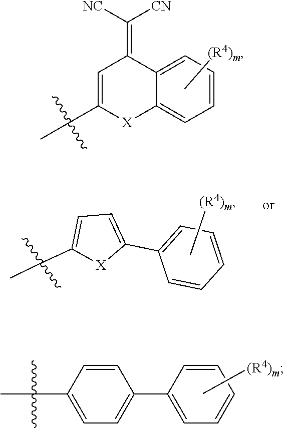

wherein
R⁴ is —C(═O)R³, —N(R³)₂, —OR³, or halo;
X is 0, S, or NR³; and
m is 0, 1, 2, or 3.

In some other embodiments, R² is:

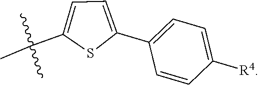

In some embodiments, R⁴ is —C(═O)H or —N(CH₃)₂.

In some embodiments, R² is:

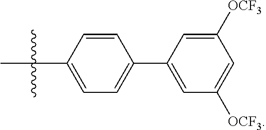 [SMILES: *c1ccc(-c2cc(OC(F)(F)F)cc(OC(F)(F)F)c2)cc1]

In various embodiments, L is —CH═CH—. In various embodiments, L has the E-configuration.

In some embodiments, L is the direct bond and R² is:

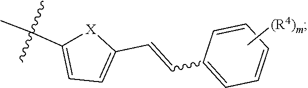

wherein
R⁴ is —C(═O)R³, —N(R³)₂, —OR³, or halo;
X is O, S, or NR³; and
m is 1, 2, or 3.

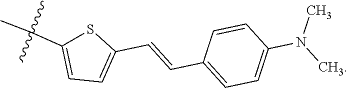 [SMILES: *c1ccc(/C=C/c2ccc(N(C)C)cc2)s1]

In some embodiments, R² is:

In various embodiments, J is OH.

In preferred embodiments, the compound is represented by Formula II:

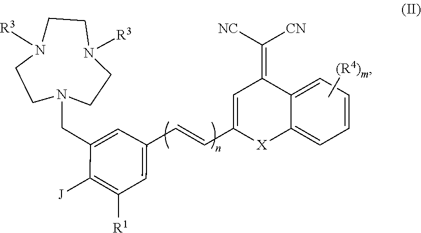

or a salt, metal complex, or combination thereof;
wherein n is 1 or 2.

In other preferred embodiments, the compound is represented by Formula III:

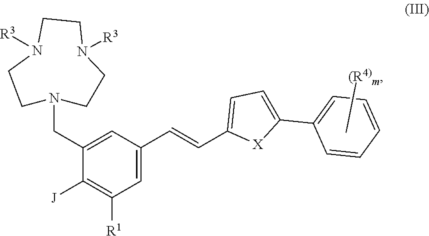

or a salt, metal complex, or combination thereof.

In yet other preferred embodiments, the compound is represented by Formula IV:

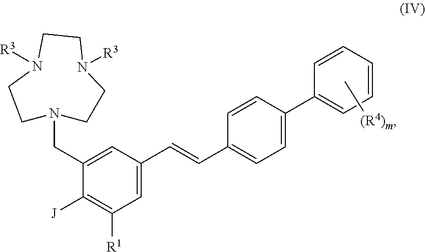

or a salt, metal complex, or combination thereof.

In additional embodiments, the compound is represented by Formula V:

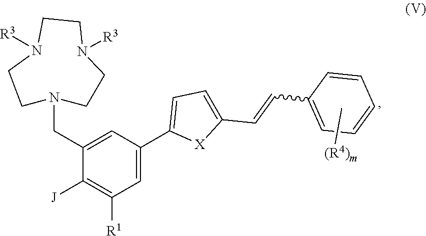

or a salt, metal complex, or combination thereof.

In various embodiments, R¹ is —CH₂N(CH₂CH₂NR³CH₂)₂. i.e.

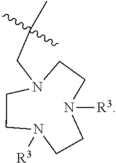

In various embodiments, —(C₁-C₆)alkyl is C₁alkyl, C₂alkyl, C₃alkyl, C₄alkyl, C₅alkyl, or C₆alkyl.

In some embodiments, the compound is: (E)-2-(2-(3-((4,7-dimethyl-1,4,7-triazonan-1-yl)methyl)-4-hydroxystyryl)-4H-chromen-4-ylidene)malononitrile (DCM-OH-1-MT); 2-(2-((1E,3E)-4-(3-((4,7-dimethyl-1,4,7-triazonan-1-yl)methyl)-4-hydroxyphenyl)buta-1,3-dien-1-yl)-4H-chromen-4-ylidene)malononitrile (DCM-OH-2-MT); (E)-2-(2-(3,5-bis((4,7-dimethyl-1,4,7-triazonan-1-yl)methyl)-4-hydroxystyryl)-4H-chromen-4-ylidene)malononitrile (DCM-OH-1-DT); 2-(2-((1E,3E)-4-(3,5-bis((4,7-dimethyl-1,4,7-triazonan-1-yl)methyl)-4-hydroxyphenyl)buta-1,3-dien-1-yl)-4H-chromen-4-ylidene)malononitrile (DCM-OH-2-DT); or a salt, metal complex, or combination thereof.

In some other embodiments, the compound is: (E)-2-((4,7-dimethyl-1,4,7-triazonan-1-yl)methyl)-4-(2-(5-(4-(dimethylamino)phenyl)thiophen-2-yl)vinyl)phenol (ZY-5-MT); (E)-2-((4,7-dimethyl-1,4,7-triazonan-1-yl)methyl)-4-(2-(5-(4-(dimethylamino)phenyl)thiophen-2-yl)vinyl)-6-methoxyphenol (ZY-5-OMe); (E)-2,6-bis((4,7-dimethyl-1,4,7-triazonan-1-yl)methyl)-4-(2-(5-(4-(dimethylamino)phenyl)thiophen-2-yl)vinyl)phenol (ZY-5-DT); (E)-4-(5-(3-((4,7-dimethyl-1,4,7-triazonan-1-yl)methyl)-4-hydroxystyryl)thiophen-2-yl)benzaldehyde (ZY-15-MT); (E)-4-(5-(3-((4,7-dimethyl-1,4,7-triazonan-1-yl)methyl)-4-hydroxy-5-methoxystyryl)thiophen-2-yl)benzaldehyde (ZY-15-OMe); (E)-4-(5-(3,5-bis((4,7-dimethyl-1,4,7-triazonan-1-yl)methyl)-4-hydroxystyryl)thiophen-2-yl)benzaldehyde (ZY-15-DT); or a salt, metal complex, or combination thereof.

In yet other embodiments, the compound is: (E)-4-(2-(3′,5′-dimethoxy-[1,1′-biphenyl]-4-yl)vinyl)-2-((4,7-dimethyl-1,4,7-triazonan-1-yl)methyl)phenol (ZY-17-MT); (E)-4-(2-(3′,5′-dimethoxy-[1,1′-biphenyl]-4-yl)vinyl)-2-((4,7-dimethyl-1,4,7-triazonan-1-yl)methyl)-6-methoxyphenol (ZY-17-OMe); (E)-4-(2-(3′,5′-dimethoxy-[1,1′-biphenyl]-4-yl)vinyl)-2,6-bis((4,7-dimethyl-1,4,7-triazonan-1-yl)methyl)phenol (ZY-17-DT); or a salt, metal complex, or combination thereof.

In additional embodiments, the compound is: (E)-2-((4,7-dimethyl-1,4,7-triazonan-1-yl)methyl)-4-(5-(4-(dimethylamino)styryl)thiophen-2-yl)phenol (ZY-12-MT); (E)-2-((4,7-dimethyl-1,4,7-triazonan-1-yl)methyl)-4-(5-(4-(dimethylamino)styryl)thiophen-2-yl)-6-methoxyphenol (ZY-12-OMe); (E)-2,6-bis((4,7-dimethyl-1,4,7-triazonan-1-yl)methyl)-4-(5-(4-(dimethylamino)styryl)thiophen-2-yl)phenol (ZY-12-DT); or a salt, metal complex, or combination thereof.

Also, this disclosure provides a metal complex of a compound disclosed herein wherein the metal of the metal complex is copper, a copper ion, or a radioisotope thereof.

In addition, this disclosure provides a method for positron emission tomography (PET) imaging amyloid-beta oligomers comprising:
a) administering to a subject a composition comprising a ⁶⁴Cu metal complex of a compound disclosed herein;
b) imaging the brain of the subject for the presence or absence of amyloid-beta oligomers by PET;
wherein the ⁶⁴Cu metal complex selectively binds to the amyloid-beta oligomers when present, thereby providing an early diagnosis for Alzheimer's disease.

This disclosure also provides a method for near infrared (NIR) imaging amyloid-beta oligomers comprising:
a) administering to a subject a composition comprising a compound disclosed herein;
b) imaging the brain of the subject for the presence or absence of amyloid-beta oligomers by NIR;
wherein the compound selectively binds to the amyloid-beta oligomers when present, thereby providing an early diagnosis for Alzheimer's disease.

Additionally, this disclosure provides a method for reducing the neurotoxicity of amyloid-beta oligomers comprising administering to a subject suffering from an early onset of Alzheimer's disease an effective amount of a composition comprising a compound disclosed herein, wherein the compound selectively binds to amyloid-beta oligomers present in the brain of the subject and reduces the neurotoxicity of amyloid-beta oligomers.

In various embodiments, the amyloid-beta oligomers are amyloid-beta-42 oligomers. In other embodiments, the amyloid-beta oligomers comprise Cu²⁺.

### EMBODIMENTS OF THE INVENTION

Embodiment 1. A compound of Formula I:

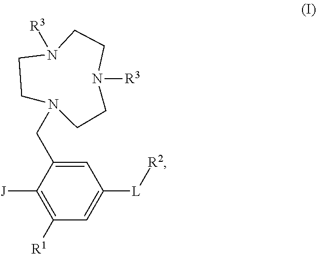

a salt, metal complex, or combination thereof, wherein optionally a metal of the metal complex is copper, a copper ion, or a radioisotope thereof;
wherein
J is OH, SH, or N(R³)₂;
L is —(CH═CH)ₙ— wherein n is 2, 1, or 0 wherein L is a direct bond between its two points of attachment when n is 0;
R¹ is —CH₂N(CH₂CH₂NR³CH₂)₂, H, or —OR³;
R² is:

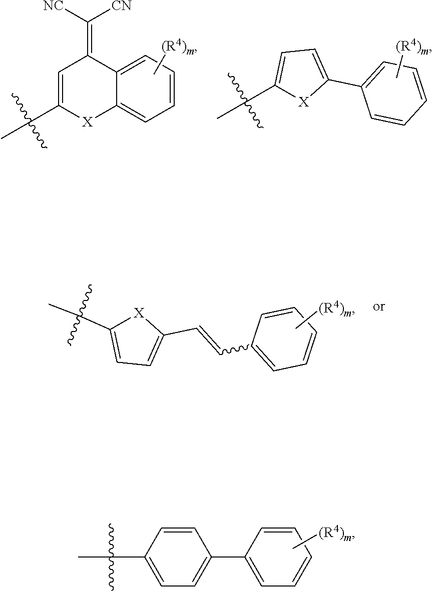

wherein
R⁴ is —C(═O)R³, —N(R³)₂, —OR³, or halo;
X is O, S, or NR³; and
m is 0, 1, 2, or 3; or
R² is a substituted phenyl; and
each R³ is independently —(C₁-C₆)alkyl or H;
   Embodiment 2. The compound of claim 1 wherein J is OH.
   Embodiment 3. The compound of claim 1 or 2 wherein L is —(CH═CH)₂— or —CH═CH—, wherein optionally L has the E-configuration.
   Embodiment 4. The compound of any one of claims 1-3 wherein R¹ is —CH₂N(CH₂CH₂NR³CH₂)₂.
   Embodiment 5. The compound of any one of claims 1-4 wherein R is:

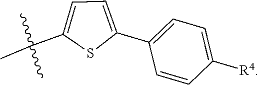

Embodiment 6. The compound of claim 5 wherein R⁴ is —C(═O)H or —N(CH₃)₂.
Embodiment 7. The compound of any one of claims 1-4 wherein L is the direct bond and R² is:

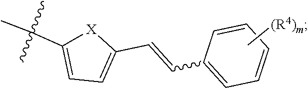

wherein
R⁴ is —C(═O)R³, —N(R³)₂, —OR³, or halo;
X is O, S, or NR³; and
m is 1, 2, or 3.
   Embodiment 8. The compound of claim 7 wherein R² is:

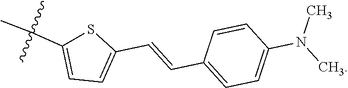 [SMILES: *c1ccc(/C=C/c2ccc(N(C)C)cc2)s1]

Embodiment 9. The compound of any one of claims 1-4 wherein R² is:

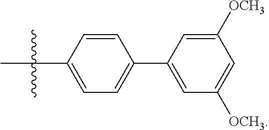 [SMILES: *c1ccc(-c2cc(OC)cc(OC)c2)cc1]

Embodiment 10. The compound of any one of claims 1-3 represented by Formula II:

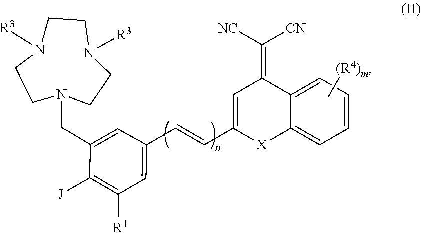

or a salt, metal complex, or combination thereof;
wherein n is 2 or 1.
Embodiment 11. The compound of any one of claims 1-3 represented by Formula III, IV, or V:

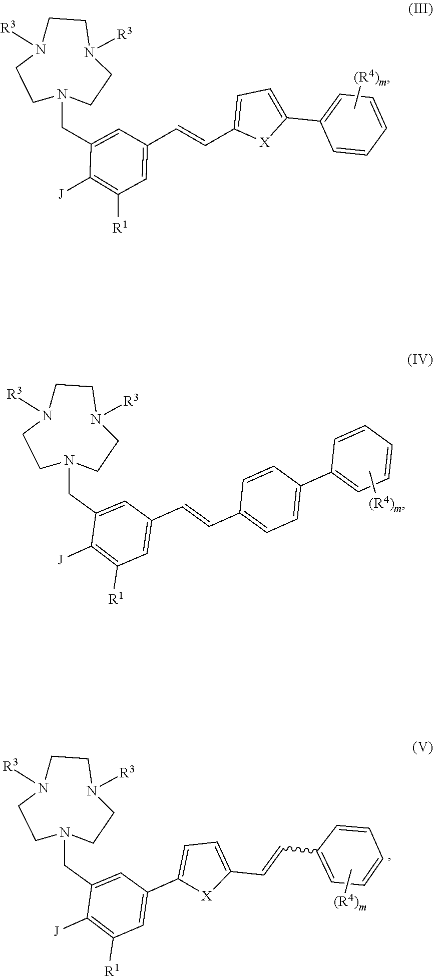

a salt, metal complex, or combination thereof.
   Embodiment 12. The compound of claim 1 wherein the compound is:

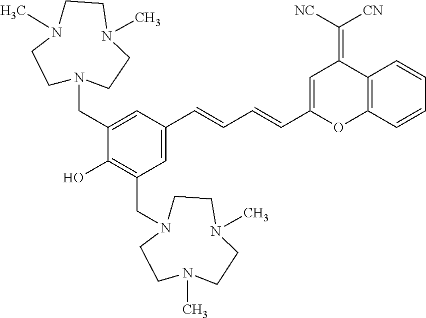 [SMILES: CN1CCN(C)CCN(Cc2cc(/C=C/C=C/C3=CC(=C(C#N)C#N)c4ccccc4O3)cc(CN3CCN(C)CCN(C)CC3)c2O)CC1]

2-(2-((1E,3E)-4-(3,5-bis((4,7-dimethyl-1,4,7-triazonan-1-yl)methyl)-4-hydroxyphenyl)buta-1,3-dien-1-yl)-4H-chromen-4-ylidene)malononitrile (DCM-OH-2-DT); any other compound disclosed herein; or a salt, metal complex, or combination thereof.

Embodiment 13. A metal complex of the compound of any one of claims 1-12 wherein the metal of the metal complex is copper, a copper ion, or a radioisotope thereof.

### Results and Discussion

Design and synthesis of the amphiphilic compounds. Inspired by the amphiphilic nature of the AR peptide, we proposed that developing compounds with amphiphilic properties targeting Aβ species could effectively treat AD. Recently, peptidomimetic-based amphiphilic compounds were shown to inhibit Aβ fibrillation process and attenuate Aβ cytotoxicity in both neuroblastoma N2a and human neuroblastoma SH-SY5Y cells. Moreover, our group successfully developed an amphiphilic small molecule, LS-4, that can serve as a therapeutic and imaging agent for Aβ oligomers in AD. LS-4 was synthesized by attaching a hydrophilic azamacrocycle, 2,4-dimethyl-1,4,7-triazacyclononane (Me₂HTACN), to a hydrophobic distyryl stilbene derivative. Interestingly, because of the incorporation of the hydrophilic TACN fragment, the binding affinity of LS-4 toward both Aβ fibrils and oligomers increased dramatically vs. the Pre-LS-4 precursor, which does not contain the Me₂TACN group. Although LS-4 showed high binding affinity to Aβ fibrils (K_{d}=58+15 nM) and Aβ oligomers (K_{d}=50+9 nM), there is no selectivity of LS-4 toward Aβ oligomers. Considering the higher neurotoxicity of Aβ oligomers and their appearance in the early stages of AD, it is beneficial to develop compounds with high affinity and selectivity toward Aβ oligomers. For this purpose, we designed a series of compounds with different amphiphilicity by adding different hydrophobic aromatic ring systems and changing the number of hydrophilic Me₂TACN groups attached to the (hetero)aromatic conjugated fragments. More specifically, we designed the TACN-Bz component of the compounds with 1) a hydroxyl group with one Me₂TACN (where R1=H, yielding the ZY−#-MT series), 2) a methoxy group with one Me₂TACN (where R1=OMe, yielding the ZY-#-OMe series), and 3) two Me₂TACN groups (where R1=Me₂TACN, giving ZY-#-DT series). In the ZY-#-OMe series, the methoxy group ortho to the hydroxyl group was introduced as its interaction with Aβ oligomers was reported previously (J. Biol. Chem. 2007, 282 (14), 10311). For the R² component of the molecules, we incorporated different aromatic ring systems (thiophene-benzene or benzene-benzene) with different substituents for their potential hydrophobic π-π interactions with the Aβ species. Based on this design approach, twelve compounds were designed for structure-activity relationships (SAR) studies and further analysis.

These 12 compounds were synthesized following a streamlined synthetic process (Scheme 1). For ZY-5-OMe/MT/DT and ZY-17-OMe/MT/DT compounds (Scheme 1A and Scheme 1B), the Horner-Wadsworth-Emmons (HWE) olefination reaction was used to form (E)-selective olefins between the MOM-protected benzyl diethyl phosphonate and different aldehyde groups under basic conditions. The free hydroxyl group was obtained after MOM deprotection using HCl, followed by a Mannich reaction with paraformaldehyde and Me₂HTACN to generate the corresponding final “OMe” or “MT” compounds. Interestingly, an additional Mannich reaction on the “MT” compounds can be performed to add a second Me₂TACN group on the unsubstituted ortho position, using paraformaldehyde and Me₂HTACN to obtain the “DT” compounds. For ZY-15-OMe/MT/DT (Scheme 1C), due to the presence of an aldehyde group on the R² component of the compound, we modified the synthesis by first forming the double bond via the HWE olefination reaction, followed by the addition of the benzaldehyde by using a Suzuki coupling reaction. In order to study the effect of the position of the double bond relative to the heterocycles, we also synthesized ZY-12-OMe/MT/DT (Scheme 1D) with similar structures to ZY-5-OME/MT/DT except for the position of the double bonds. Due to the structural differences, the synthetic steps for ZY-12-OMe/MT/DT were modified accordingly. First, a Suzuki coupling was performed between MOM-protected boron pinacol esters and thiophene bromide, followed by the HWE olefination reaction to synthesize the second intermediate containing the double bond. The MOM deprotection and the Mannich reaction were performed to obtain the final “OMe” or “MT” compounds. Similar to the synthesis of other “DT” compounds, ZY-12-DT was synthesized through an additional Mannich reaction.

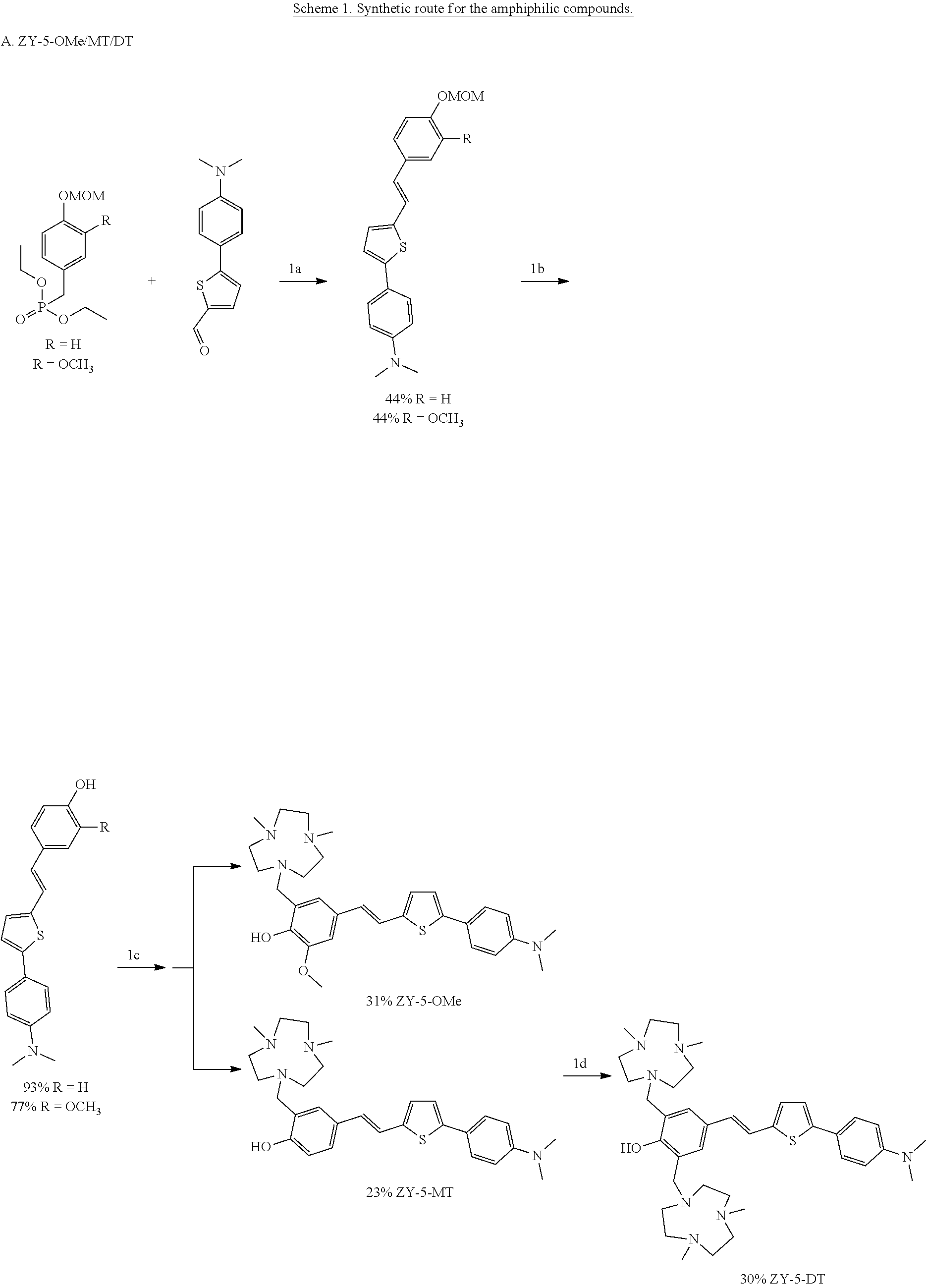

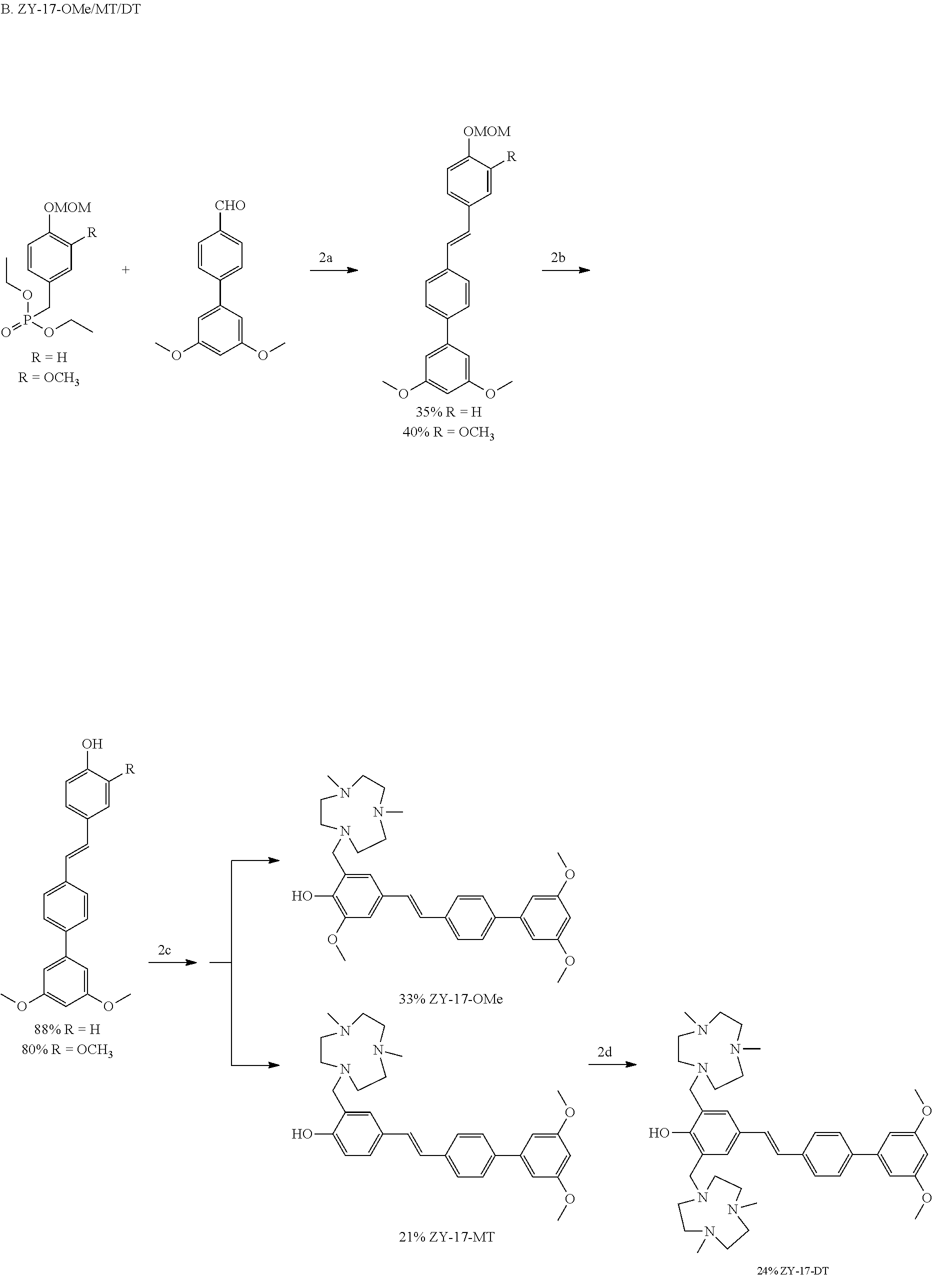

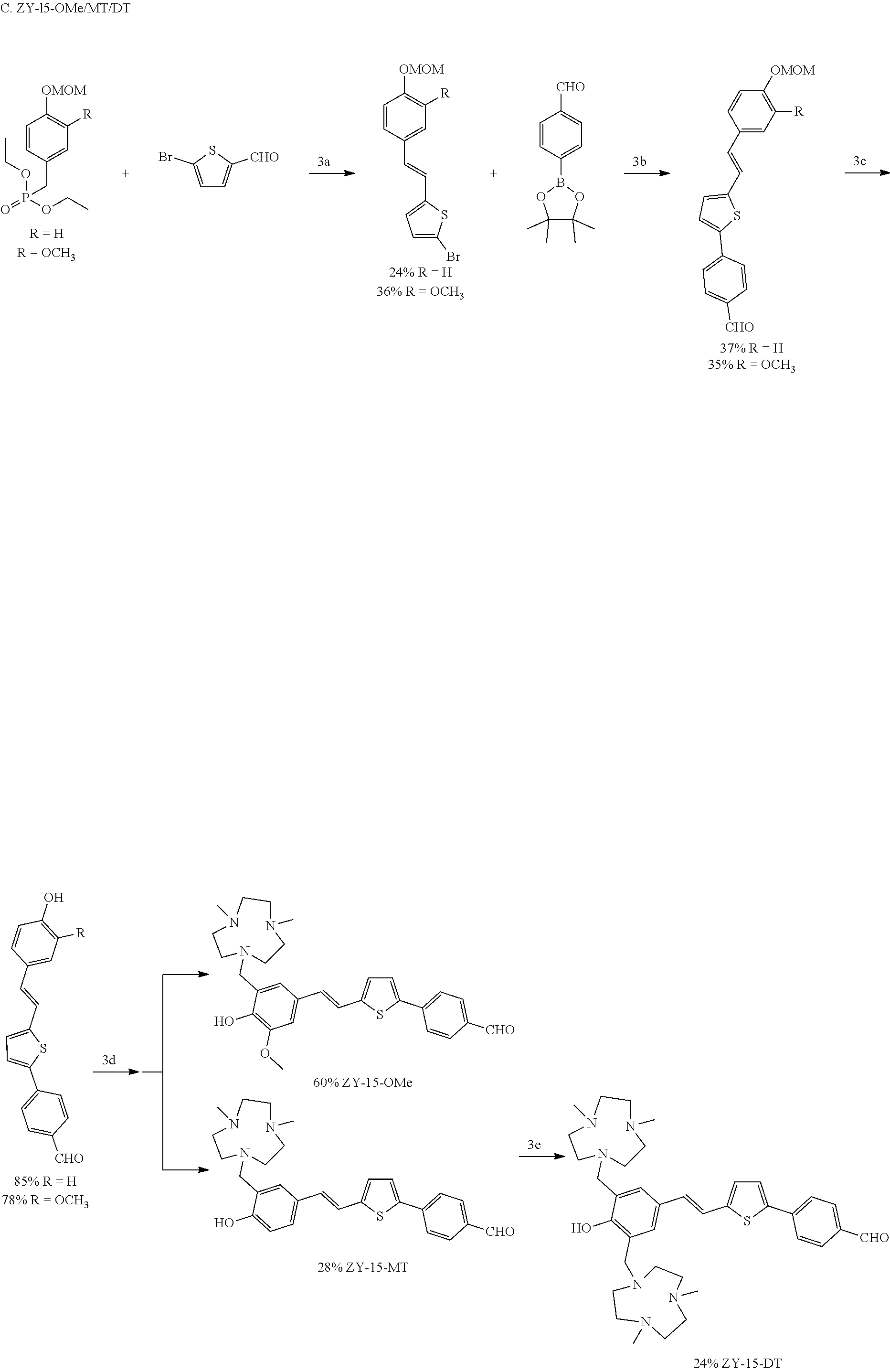

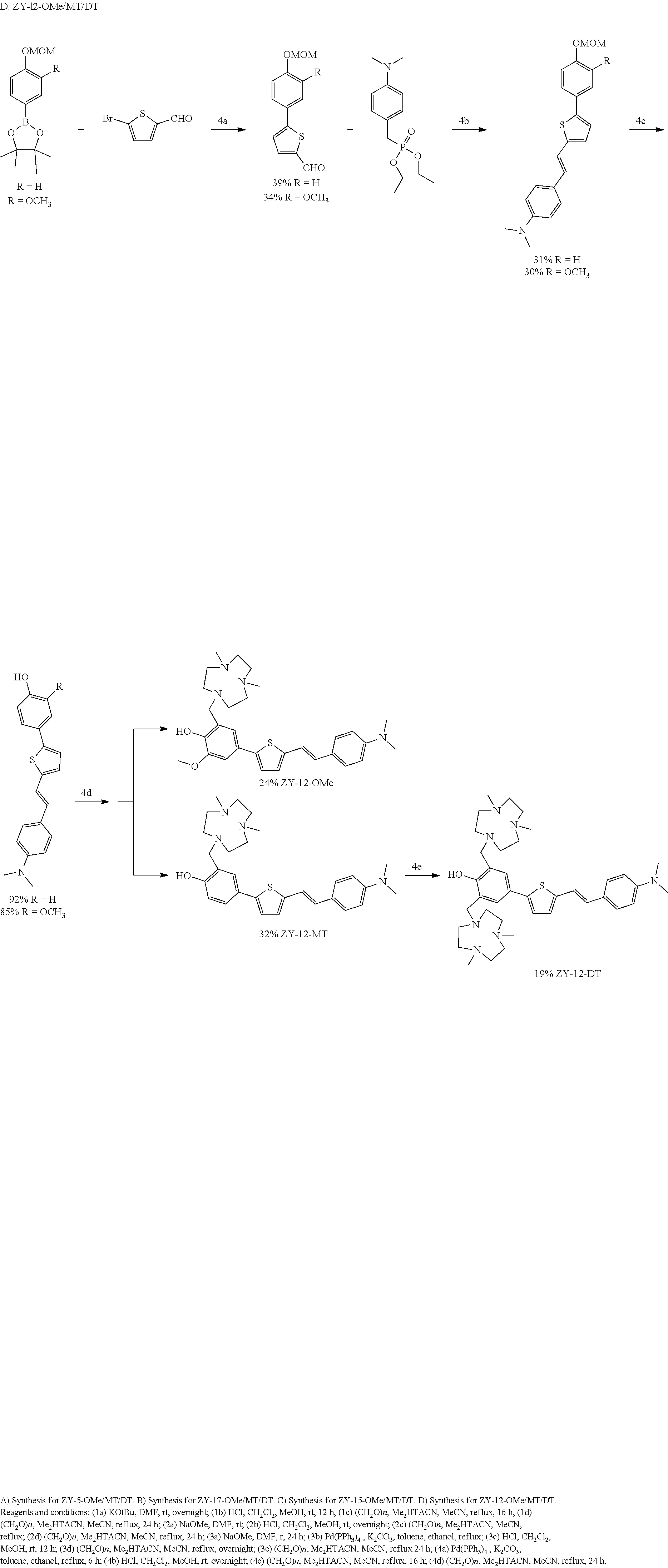

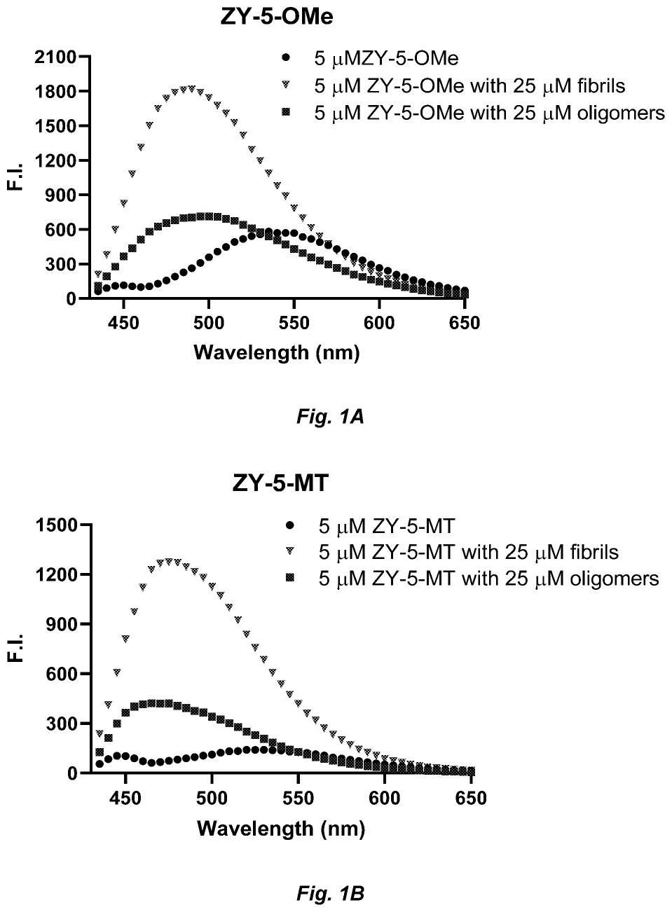

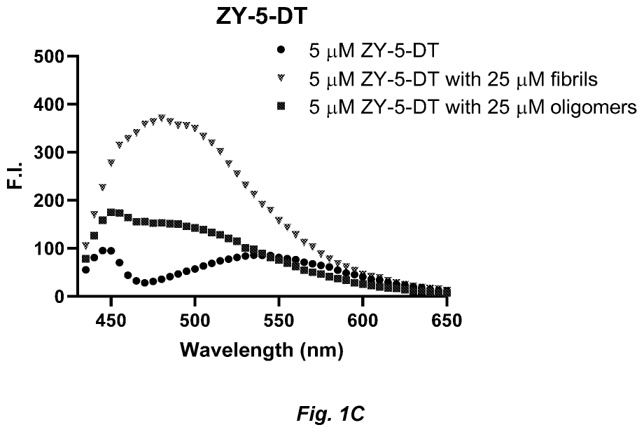

Fluorescence binding assays. To investigate whether these amphiphilic compounds can interact with different Aβ species, we recorded the fluorescence intensity changes in the absence and presence of Aβ₄₂ fibrils and oligomers, respectively prepared as previously reported (Neurosci. 2001, 24 (4), 219). These compounds, such as the ZY-5 series exhibit a fluorescence turn-on effect when binding to Aβ species, along with a dramatic blue shift of the emission wavelength (FIG. 1). The enhanced fluorescence intensity and blue shift are possibly due to the restriction in a rotation of the aromatic rings and the changes of hydrophobicity at the Aβ binding sites.

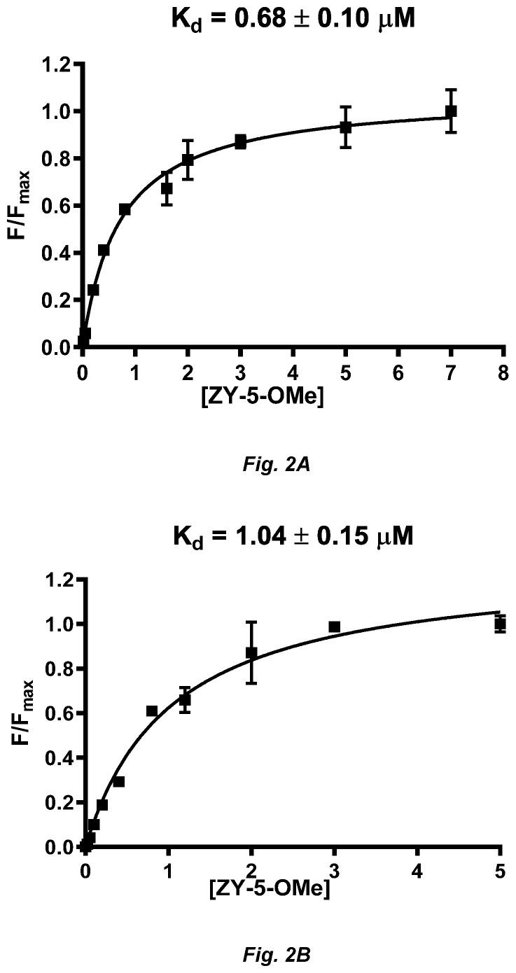

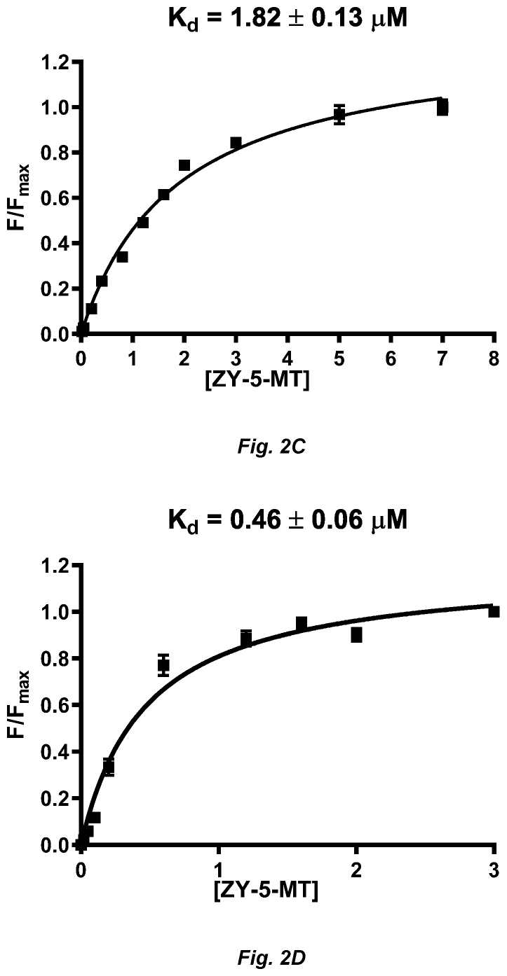

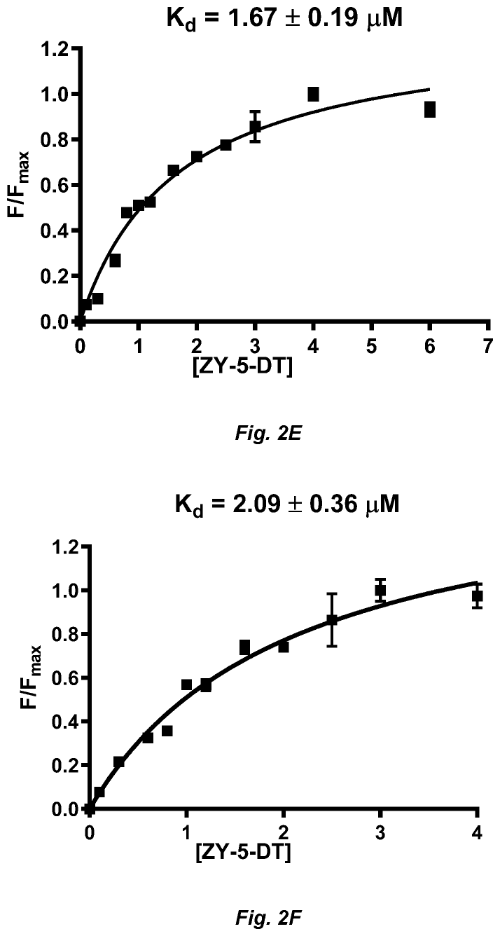

Inspired by the observed fluorescence turn-on effect, we then measured the binding constants (K_{d} values) toward Aβ₄₂ fibrils and oligomers using fluorescence saturation assays. Representative K_{d} curves and K_{d} values obtained for the ZY-5 series are shown in FIG. 2. K_{d} values for all twelve compounds provide a direct comparison of their interaction with fibrils and oligomers (Table 1). It is worth noting that some compounds have high binding affinity to Aβ₄₂ oligomers evidenced by the low K_{d} values (e.g., ZY-17-OMe: K_{d}=0.12 μM, ZY-12-OMe: K_{d}=0.32 μM. These values are comparable to the K_{d} values of other reported oligomer-selective probes: CRANAD-102 (7.5+10 nM), BD-Oligo (K_{d}=0.4 μM), and F-SLOSH (K_{d}=0.66 μM). More interestingly, six of the compounds (ZY-5-MT, ZY-12-OMe, ZY-12-DT, ZY-15-OMe, ZY-17-OMe, and ZY-17-DT) showed relatively lower K_{d} values for Aβ oligomers than Aβ fibrils, indicating their higher binding affinity to oligomers over fibrils. Except for ZY-5-OMe, the other compounds bearing methoxy groups ZY-12-OMe, ZY-15-OMe, and ZY-17-OMe showed higher affinity toward Aβ oligomer suggesting that the methoxy group might increase the compounds' interactions with Aβ oligomers. Moreover, the ZY-5 and ZY-12 series bind to Aβ species differently despite their similar chemical structures. More specifically, ZY-5-MT showed a high affinity to both fibrils and oligomers, with an increased selectivity to Aβ oligomers (of 3.9 times higher than that for Aβ fibrils). In contrast, ZY-12-MT exhibited low affinity to both fibrils and oligomers. Taken together, we consider the different structures developed along with the K_{d} values will be beneficial for developing more oligomer-specific compounds.

TABLE 1
Summary of K_{d} values of amphiphilic compounds
binding to Aβ₄₂ fibrils and oligomers.

| Compounds | Fibrils (μM) | Oligomers (μM) |
|---|---|---|
| ZY-5-OMe | 0.68 ± 0.10 | 1.04 ± 0.15 |
| ZY-5-MT | 1.82 ± 0.13 | 0.46 ± 0.06 |
| ZY-5-DT | 1.67 ± 0.19 | 2.09 ± 0.36 |
| ZY-12-OMe | 0.39 ± 0.04 | 0.32 ± 0.04 |
| ZY-12-MT | 4.19 ± 0.87 | 6.27 ± 1.01 |
| ZY-12-DT | 1.38 ± 0.24 | 0.67 ± 0.15 |
| ZY-15-OMe | 1.24 ± 0.18 | 0.47 ± 0.13 |
| ZY-15-MT | 0.53 ± 0.03 | 2.03 ± 0.11 |
| ZY-15-DT | 1.27 ± 0.10 | 4.55 ± 0.32 |
| ZY-17-OMe | 0.18 ± 0.02 | 0.12 ± 0.02 |
| ZY-17-MT | 0.94 ± 0.07 | 2.08 ± 0.34 |
| ZY-17-DT | 2.25 ± 0.46 | 1.03 ± 0.20 |

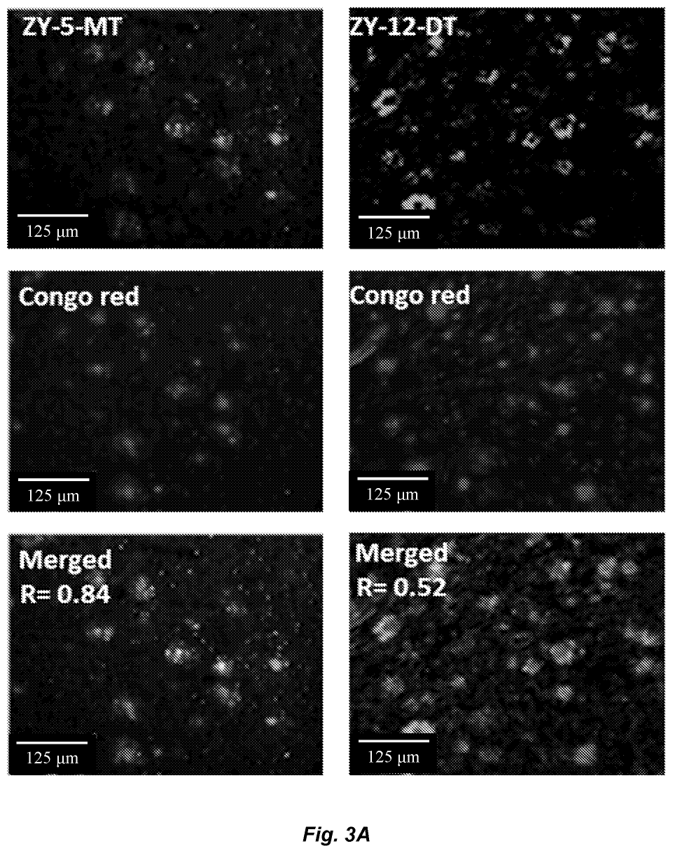

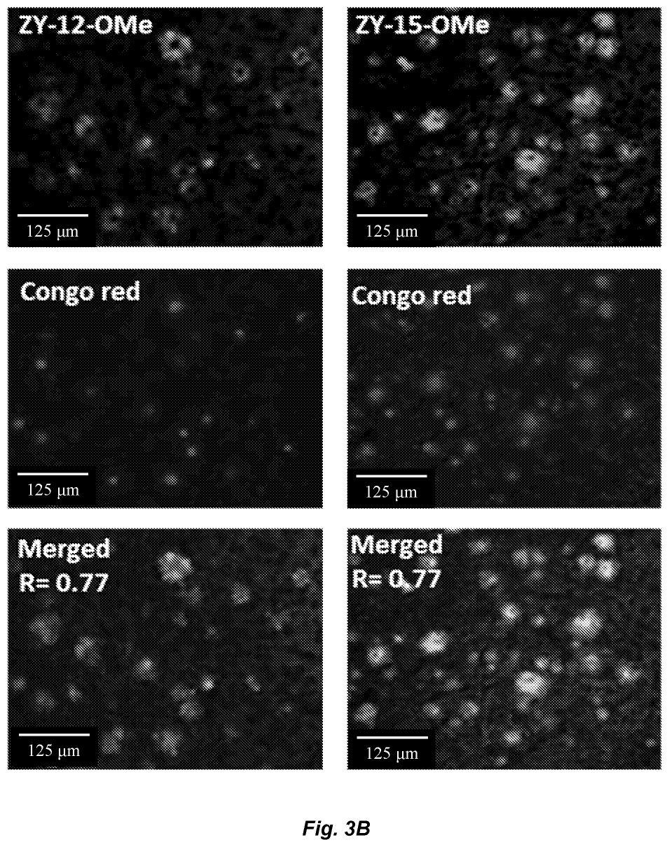

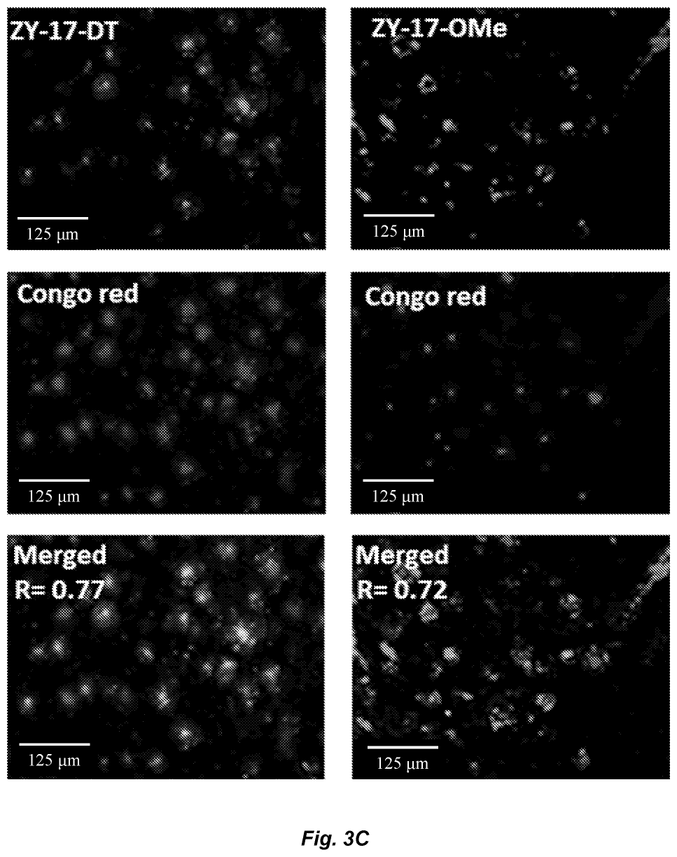

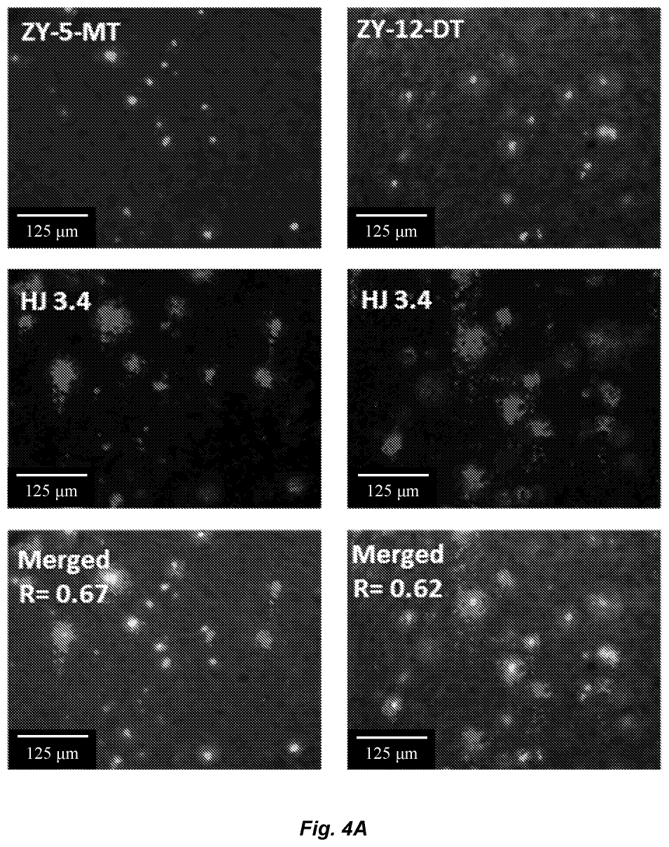

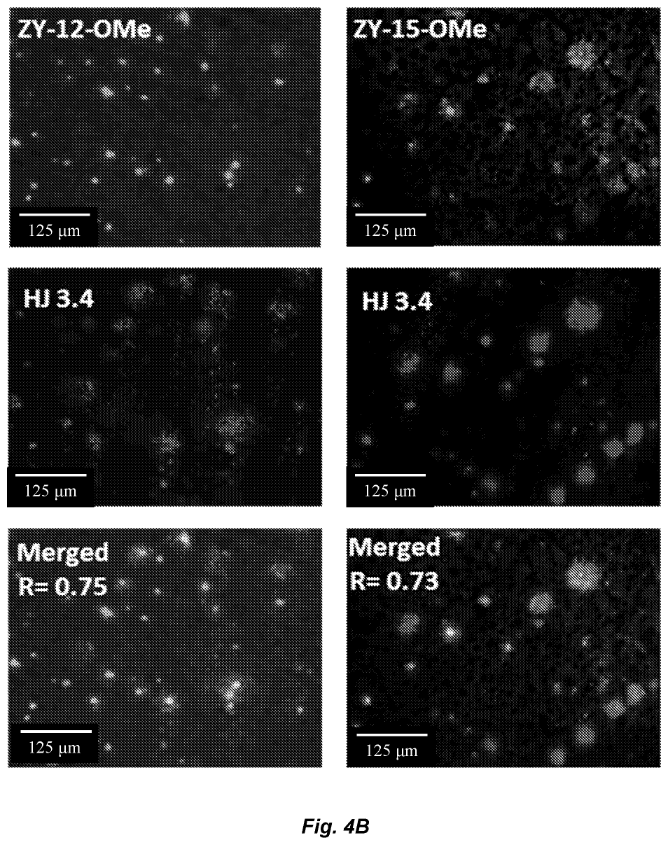

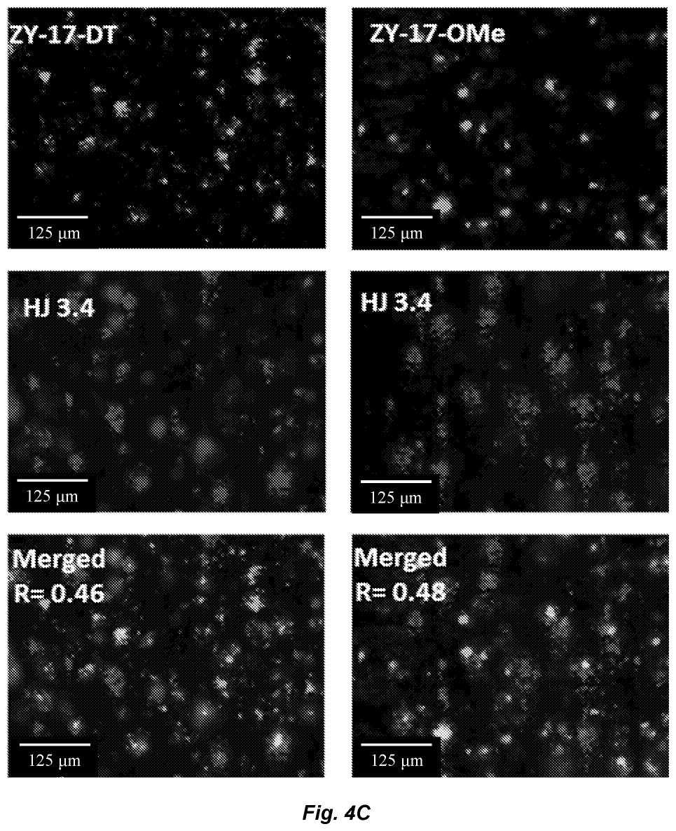

Fluorescence Imaging of 5×FAD Mouse Brain Sections. In order to confirm that the compounds can also bind to native Aβ species, brain sections collected from 9-month-old 5×FAD transgenic mice have been employed in the fluorescence imaging studies. 5×FAD transgenic mice were shown to develop AD pathologies at a young age, and aggregated Aβ species were commonly observed in brain sections from 5×FAD transgenic mice. The brain sections were first incubated with our compounds, followed by Congo red (CR), which is a well-established fluorescent probe that can bind to Aβ plaques. The treated brain sections were then imaged using fluorescence microscope. The six compounds with high binding affinities for Aβ₄₂ oligomers based on K_{d} measurements showed well-defined fluorescence staining signals and good colocalization with Congo Red (CR), as indicated by the Pearson's correlation coefficients (FIG. 3). Interestingly, these compounds tend to bind preferentially to the periphery of the amyloid plagues, while CR binds to the dense core region of the plaques. Immunostaining with the CF594-conjugated HJ 3.4 antibody (CF594-HJ3.4) was also performed to confirm that these six compounds can bind to Aβ species specifically in the brain sections (FIG. 4). Moreover, the Log D values of the compounds were also measured by octanol-PBS partition assays. These twelve compounds exhibit similar Log D values ranging from 0.8 to 1.2 (Table 2), indicating their ability to cross the blood-brain barrier (BBB) for potential in vivo applications.

TABLE 2
Summarized optical properties.

| Compounds | λₑₓ (nm) | λₑₘ (nm) |
|---|---|---|
| ZY-5-OMe | 385 | 545 |
| ZY-5-MT | 355 | 530 |
| ZY-5-DT | 390 | 545 |
| ZY-12-OMe | 370 | 540 |
| ZY-12-MT | 380 | 445 |
| ZY-12-DT | 380 | 540 |
| ZY-15-OMe | 405 | 500 |
| ZY-15-MT | 405 | 500 |
| ZY-15-DT | 405 | 590 |
| ZY-17-OMe | 335 | 470 |
| ZY-17-MT | 335 | 470 |
| ZY-17-DT | 335 | 520 |

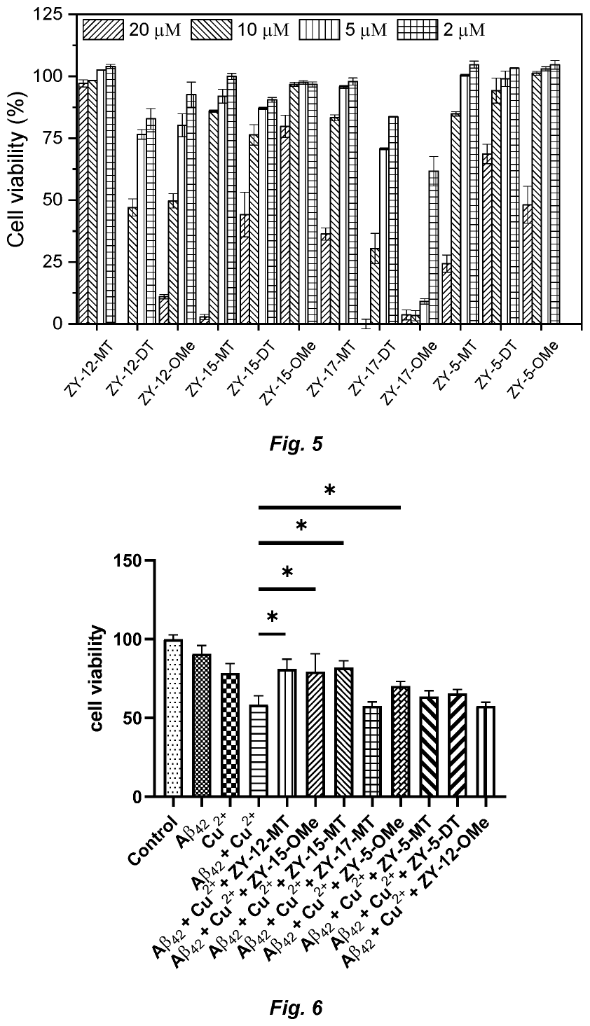

Modulation of Cu²⁺-Aβ₄₂ neurotoxicity. After confirming these amphiphilic compounds can bind to Aβ species both in vitro and ex vivo, we next investigated whether these compounds could attenuate the toxicity of the Cu-AD species, as the Cu²⁺ ions were reported to promote the formation of neurotoxic soluble Aβ₄₂ oligomers. Firstly, the Alamar blue cell viability assay was used to measure the cytotoxicity of the compounds at different concentrations ranging from 2 μM to 2 μM in mouse neuroblastoma N2a cells (FIG. 5). Some compounds (ZY-12-MT, ZY-15-MT, ZY-15-OMe, ZY-17-MT, ZY-5-MT, ZY-5-DT, and ZY-5-OMe) exhibited no significant cytotoxicity (indicated by >80% cell viability) up to 1 μM. Hence, these compounds are good candidates for the Cu²⁺-Aβ₄₂-induced cytotoxicity studies (see below). For ZY-12-OMe, the compound was quite toxic even at 10 μM (cell viability less than 50%), yet at 5 μM it exhibited less cytotoxicity (more than 75% cell viability). Considering its high binding affinity to both Aβ fibrils and oligomers, we also further tested its ability to alleviate Cu²⁺-Aβ₄₂-induced toxicity. Unfortunately, some of the compounds showed a high binding affinity to oligomers, such as ZY-12-OMe, ZY-12-DT, ZY-17-DT, and ZY-17-OMe, exhibited higher citotoxicity than others. Additionally, ZY-17-OMe, which has the highest binding affinity to oligomers, showed the highest toxicity. The underlying mechanism is unclear, but we propose that these compounds might perform similarly to the toxic oligomers that bind to some cell membrane receptors or insert into the membrane lipids to form porous channels.

Since our compounds exhibit high binding affinity to both Aβ fibrils and oligomers and contain TACN group(s) that can potentially bind to Cu and disrupt the Cu-Aβ₄₂ interaction, it is essential to study their roles in alleviating Cu²⁺-Aβ₄₂-induced toxicity. As mentioned above, compounds that are not cytotoxic at 10 μM, including ZY-12-MT, ZY-15-MT, ZY-15-OMe, ZY-17-MT, ZY-5-MT, ZY-5-DT, ZY-5-OMe, and ZY-12-OMe, were chosen for this study. Firstly, during the control studies, we observed that monomeric Aβ₄₂ led to negligible neurotoxicity. Nevertheless, in the presence of both Cu²⁺ and monomeric Aβ₄₂, there was a significant cell death, which is likely due to the Cu²⁺ associated neurotoxic Aβ₄₂ oligomers formation. We observed compounds ZY-12-MT, ZY-15-OMe, ZY-15-MT, and ZY-5-OMe could significantly increase cell viability, while the other ones could not reduce the neurotoxicity of the Cu²⁺-Aβ₄₂ species (FIG. 6). Interestingly, when compared to ZY-5-MT, even though ZY-15-OMe is less selective toward Aβ oligomers, it can alleviate Cu²⁺-Aβ₄₂-induced toxicity, likely due to its interaction with Tyr10 via hydrogen bond and 7r-7c interactions. According to the docking results, ZY-12-MT, ZY-15-OMe, ZY-15-MT, and ZY-5-OMe interact with Aβ₄₂ tetramers mainly through residues His6, Asp7, Tyr10 (via hydrogen bonds), and Tyr10 (via π-π interactions). Some reports have also shown that His6, Asp7, and Tyr10 are potentially involved in the Cu-Aβ interactions, which explains why the compounds are able to attenuate the Cu-Aβ toxicity.

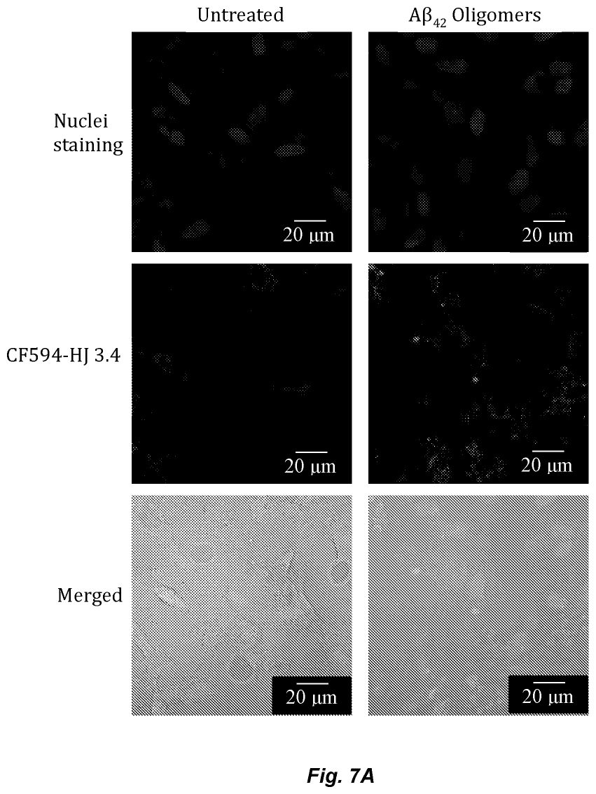

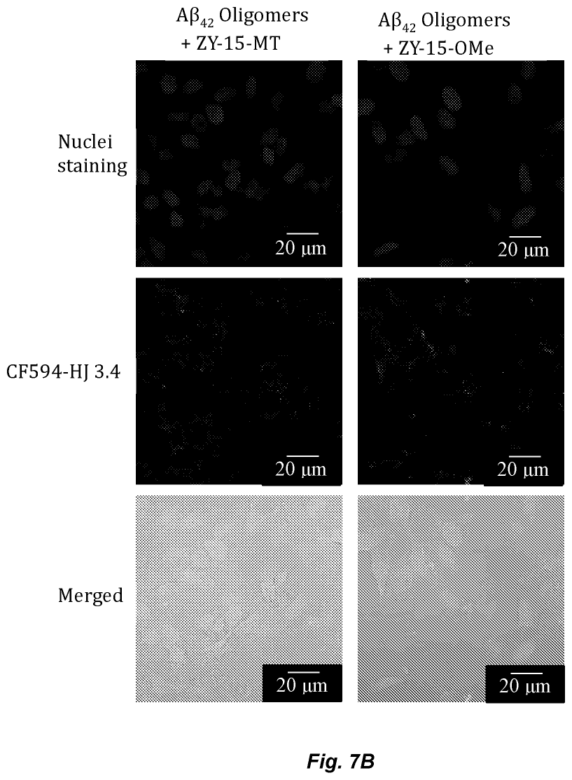

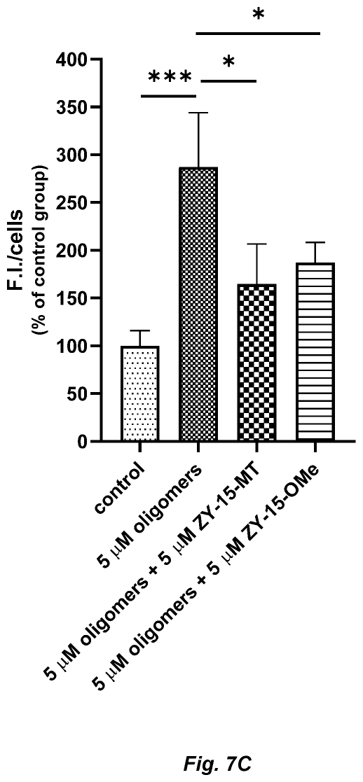

Modulating the Aβ-cell membrane interactions. Encouraged by the ability of the developed amphiphilic compounds to attenuate the neurotoxicity of Cu-Aβ species, we sought out to study the possible molecular mechanisms for this beneficial effect. The Aβ oligomers were reported to interact with cell membranes in various ways, such as binding to receptors on cell membranes, inserting into membranes, or even showing cellular uptake via endocytosis. The abnormal interactions between Aβ oligomers and neuron cells, which could disrupt the neuronal ion homeostasis and neuron cell membrane integrity, might be why Aβ oligomers are highly neurotoxic. Moreover, molecules that can disrupt interactions between oligomers and cell membranes are promising candidates for drug development. Thus, we proceeded to probe the interactions of the Aβ₄₂ oligomers with SH-SY5Y cellular membranes, in the absence and presence of the compounds, via confocal microscopy. Since ZY-15-MT and ZY-15-OMe can rescue cell viability to a higher extent and exhibit a higher affinity for Aβ oligomers than ZY-12-MT, they were chosen for this experiment. SH-SY5Y cells were treated with Aβ oligomers or a combination of Aβ oligomers and compounds for 24 h, followed by the immunofluorescence staining with the CF594-labeled anti-AD antibody HJ 3.4 (FIG. 7) and nuclei staining, shown in the red and blue channels, respectively. Compared to the untreated group, the Aβ oligomers were found mainly bound to cell membranes. Moreover, in the presence of both ZY-15-MT and ZY-15-OMe, there were fewer numbers of Aβ oligomers bound to the cell membranes, as shown in the red channel (FIG. 7a). Interesting, ZY-15-MT is able to decrease the numbers of the Aβ oligomers binding to the cell membranes to a larger extent (about 50%, FIG. 7b), even though ZY-15-MT shows a lower affinity to oligomers than ZY-15-OMe. To understand this unusual behavior, we posited that the Aβ oligomers would start the fibrilization process when incubated in the cell media. ZY-15-MT, which exhibits a higher affinity to Aβ fibrils than ZY-15-OMe, might be able to bind to the more aggregated Aβ species and can also decrease their interactions with cell membranes.

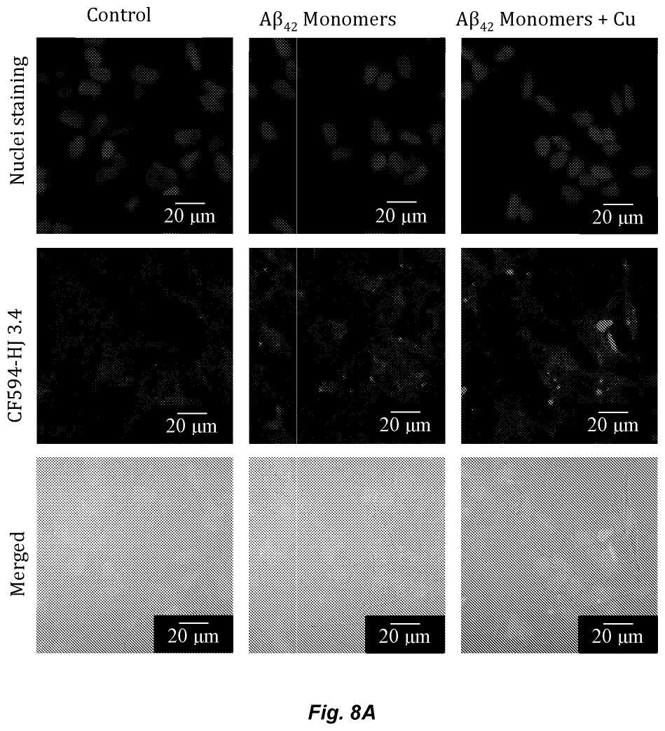

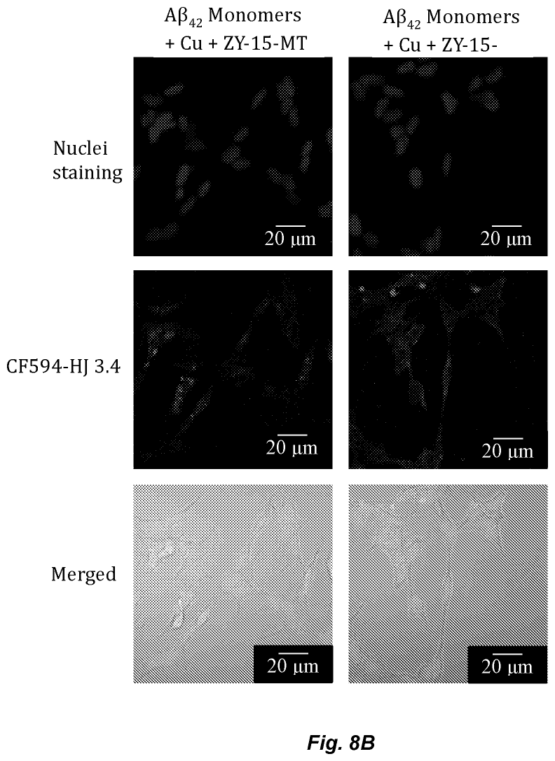

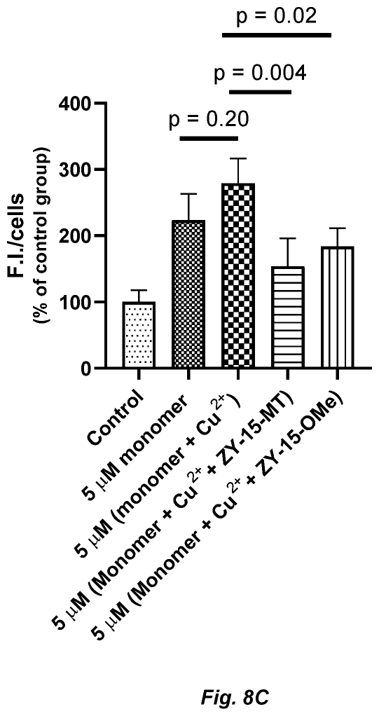

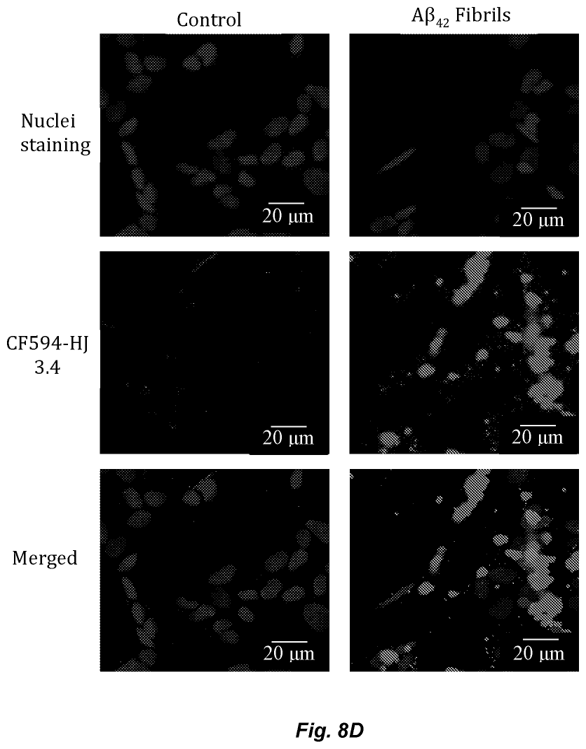

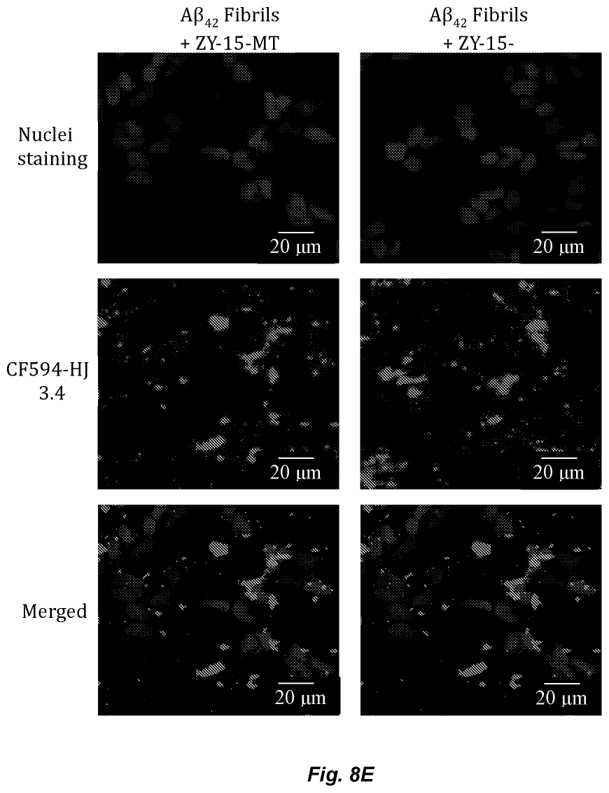

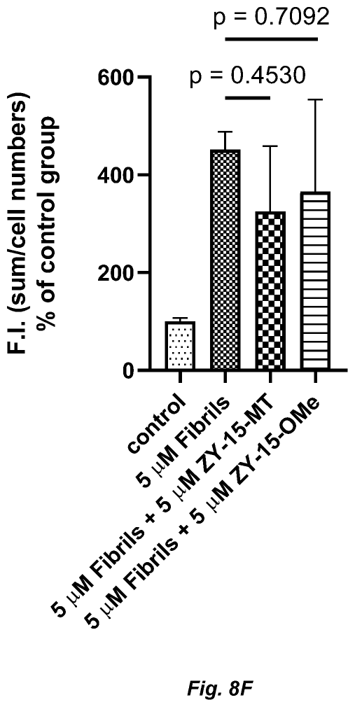

Therefore, proceeded to probe if our compounds could prevent the binding of Aβ fibrils to cell membranes. When SH-SY5Y cells were treated with Aβ fibrils or a combination of fibrils and compounds for 24 h, both ZY-15-MT and ZY-15-OMe were not able to significantly decrease the numbers of the Aβ fibrils binding to cell membranes, while ZY-15-MT seems to decrease the interactions between Aβ fibrils and SH-SY5Y more than ZY-15-OMe (FIG. 8c, FIG. 8d). These results further suggest that both ZY-15-MT and ZY-15-OMe are more potent to alleviate the neurotoxicity of the Aβ oligomeric species. Given these encouraging results, we moved to study if the amphiphilic compounds can further control the interactions between Cu-Aβ species and cell membranes. Firstly, when SH-SY5Y cells were treated with Aβ monomers for 48 h, the aggregated Aβ species interacting with cell membranes were observed (FIG. 8a). Moreover, in the presence of Cu², even though there were only slightly increased interactions with membranes, larger aggregated AR species were observed comparing to the group only with Aβ monomers treated (FIG. 8a and FIG. 8b). However, these aggregates are still smaller than the insoluble fibrils (FIG. 8c, FIG. 8d), indicating they are still soluble oligomeric Aβ species. Excitingly, in the presence of either compound, the Cu-Aβ species showed significantly decreased interactions with cell membranes (FIG. 8a and FIG. 8b), which might explain why the compounds can attenuate the toxicity of the Cu-Aβ aggregates. Taken together, we believe these findings suggest the developed amphiphilic compounds are neuroprotective by controlling the interactions between the Aβ oligomers and cell membranes, which we consider is a novel approach for the development of AD therapeutics.

In summary, we designed and synthesized twelve amphiphilic compounds and studied their binding affinity toward Aβ₄₂ species in vitro and ex vivo. More importantly, according to molecular docking studies, we found that targeting amino acids His6 and Asp7 might increase the binding affinity and selectivity toward Aβ₄₂ oligomers. Moreover, according to cellular studies, we found that compounds with a higher binding affinity toward Aβ₄₂ oligomers exhibit higher neurotoxicity, and most of them interact mainly with the amino acids His6 and Asp7. The only exception is compound ZY-15-OMe, which maintains its selectivity to Aβ₄₂ oligomers via hydrogen bonds and π-π interactions with Tyr10, and while exhibiting lower inherent cell toxicity, it was able to alleviates the Cu-Aβ₄₂ neurotoxicity. Finally, confocal microscopy imaging studies show that both ZY-15-MT and ZY-15-OMe were able to decrease the interactions between Aβ oligomers and SH-SY5Y cell membranes, demonstrating their ability to target Aβ oligomers. These studies strongly suggest that such amphiphilic compounds could be an effective strategy to differentiate between Aβ oligomers and Aβ fibrils. These encouraging results will help design Aβ oligomer-selective lead compounds to can be used for AD therapeutic agent development.

Synthesis of Novel Dicyanomethylene Derivatives and their Applications as Dual Imaging Agents for Alzheimer's Disease.

Although the pathogenesis of AD is still not clear, two misfolded protein deposits, amyloid-β (Aβ) aggregates and neurofibrillary tangles (NFT) are considered as the pathologic hallmarks of AD. More importantly, soluble Aβ oligomers were found to be the most neurotoxic species directly implicated in synapse loss and neuronal injury. Since the Aβ oligomer aggregates appear dominantly in the early stage of the disease, the soluble Aβ oligomers are attractive targets for early diagnosis of AD. Our group has reported an effective strategy that shows amphiphilic molecules exhibit high binding affinity to Aβ oligomers (Journal of the American Chemical Society 2021, 143 (27), 10462-10476). Inspired by these results, a series of new amphiphilic compounds were designed by attaching one or two of the hydrophilic azamacrocycle 2,4-dimethyl-1,4,7-triazacyclononane (Me2TACN, shown in Chart 2a, b), to a hydrophobic fluorophore dicyanomethylene-4H-pyran fragment (DCM, Chart 2). With the addition of the hydrophilic moieties, the constructed compounds have shown increased selectivity toward Aβ oligomers. Moreover, the DCM-OH-2-DT compound, which has two conjugated double bonds and two Me2TACN groups ortho to the phenol group, exhibits the maximum emission wavelength of 730 nm when binding to various Aβ aggregates (AD fibrils and oligomers). As the result, this is an ideal candidate for in vivo NIR fluorescence imaging. Finally, due to the rapid complexation of Cu by the Me2TACN chelating moiety (Journal of the American Chemical Society 2017, 139 (36), 12550-12558; Inorg Chem 2017, 56 (22), 13801-13814), we can perform PET imaging studies with the ⁶⁴Cu-radiolabeled complexes (Chart 2c).

Herein is disclosed the synthesis of novel amphiphilic DCM derivatives and their applications as dual imaging agents in both NIR fluorescence and PET imaging for the early diagnosis of AD. The developed imaging agents have two potential applications. On one hand, the newly developed compounds can chelate to ⁶⁴Cu isotope to form PET imaging tracers. ⁶⁴Cu-radiolabeled tracers are attractive alternatives to ¹⁸F-labeled tracers due to the relative longer half-life time of ⁶⁴Cu (12.7 h), resulting in a wider application in the clinic since these imaging agents can be shipped throughout the country, while also being more effective in the early diagnosis of AD.

On the other hand, the developed compounds can also be used for NIR fluorescence imaging. In small animal studies, which are essential for the development of therapeutics, NIR imaging has several advantages, such as the lack of need for using radioactive nuclei, expensive equipment, or a time-consuming data acquisition process. At the same time, this noninvasive technique can provide enough light for deep tissue penetration and a real-time imaging process. Therefore, the developed compounds can be used to monitor the in vivo response to therapies in different small animal models, assisting the discovery of potential drug candidates.

Chart 2. a), b) Molecular structures for DCM-based compounds. c) Me2TACN-based ⁶⁴Cu labeled complexes.

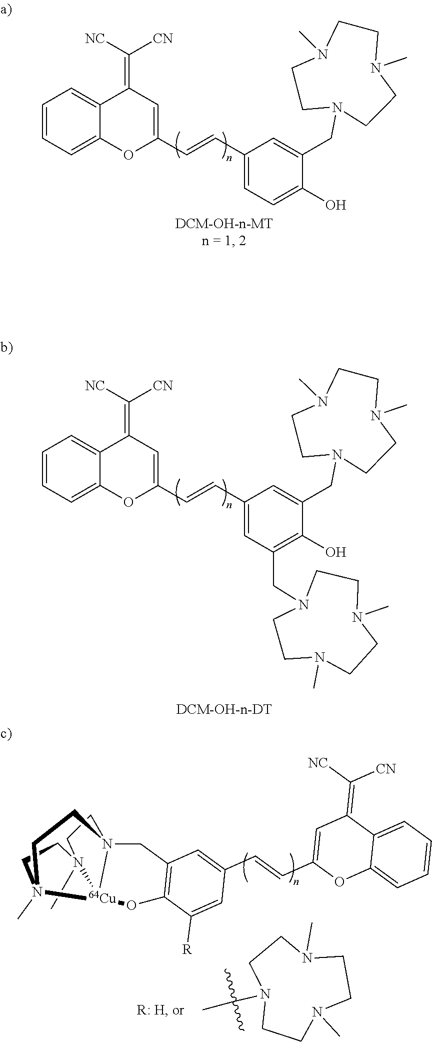

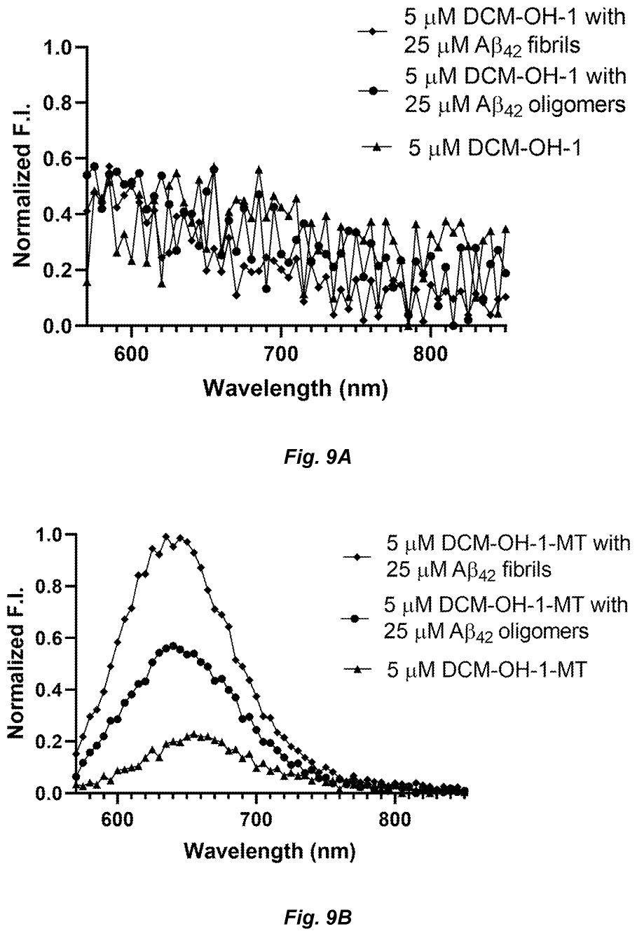

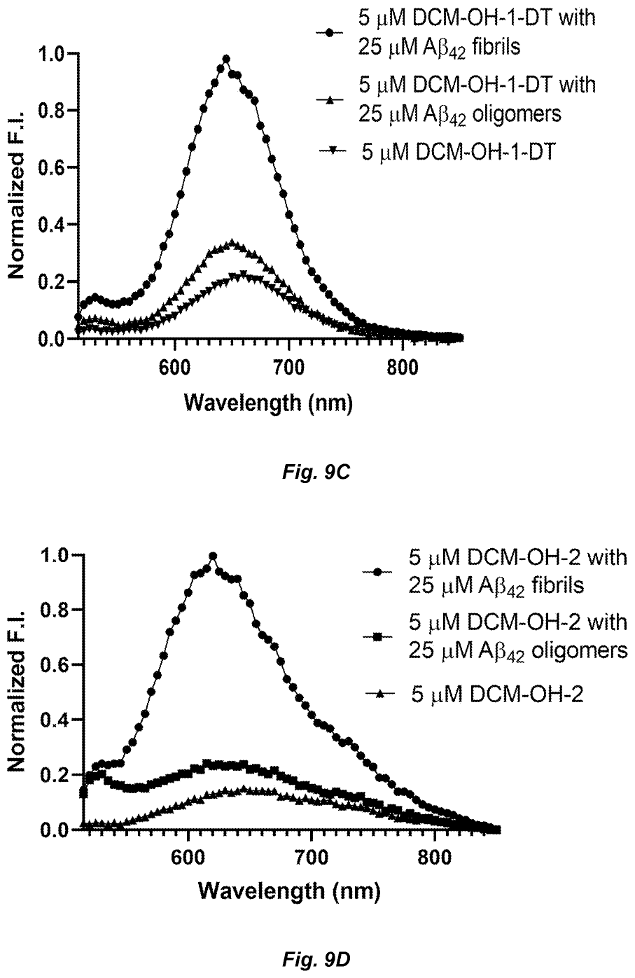

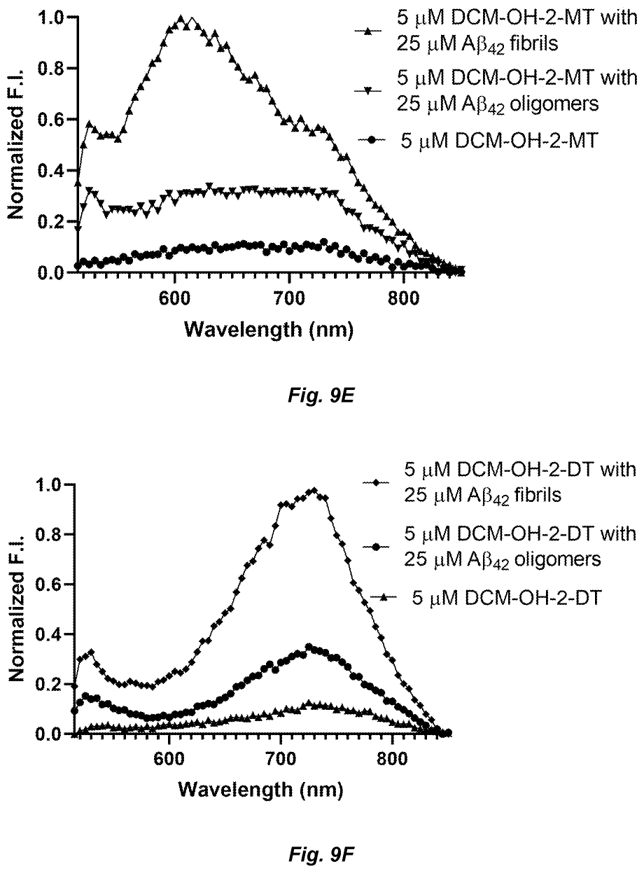

Fluorescence binding assays. The developed compounds exhibit significant fluorescence enhancement when binding to Aβ₄₂ fibrils and oligomers (FIG. 9). Due to the fluorescence turn-on effect, we obtained the binding affinity (K_{d} values) of the compounds towards both Aβ₄₂ fibrils and oligomers by performing fluorescence saturation assays (FIG. 9 and Table 3). It is worth noting that these compounds show low micromolar K_{d} values, indicating that their strong interactions with Aβ₄₂ fibrils and oligomers. DCM-OH-2 has the highest binding affinity, with a K_{d} value of 80 nM to Aβ₄₂ fibrils and a K_{d} value of 120 nM to Aβ₄₂ oligomers. Importantly, with a hydrophilic Me₂TACN groups attached, the selectivity of the compounds towards Aβ₄₂ oligomers increased. The compound DCM-OH-2-MT has three times higher binding affinity towards Aβ₄₂ oligomers over fibrils, which demonstrates the importance of including hydrophilic fragments for the development of oligomers-selective compounds. Furthermore, Log D values of the compounds were obtained via PBS-Octanol partition coefficient measurement. The compounds have appreciable lipophilicity with Log D values vary from 1 to 2 (Table 4), which indicates their abilities to cross the blood-brain barrier (BBB).

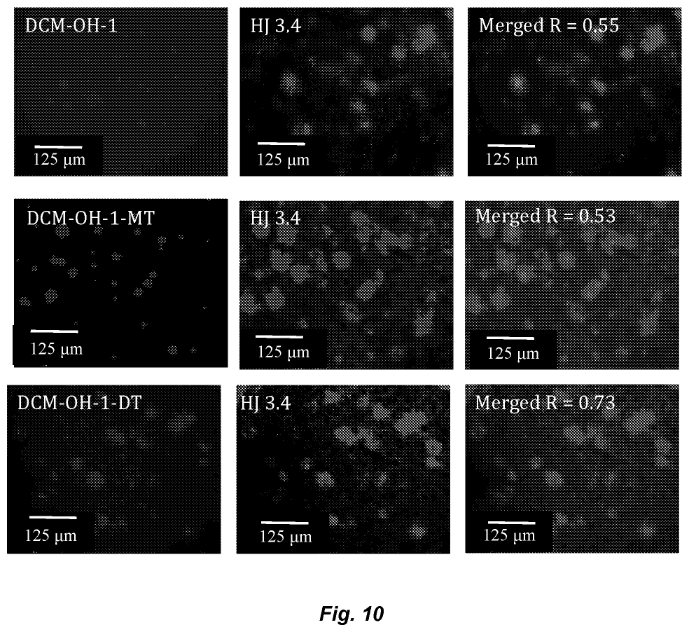

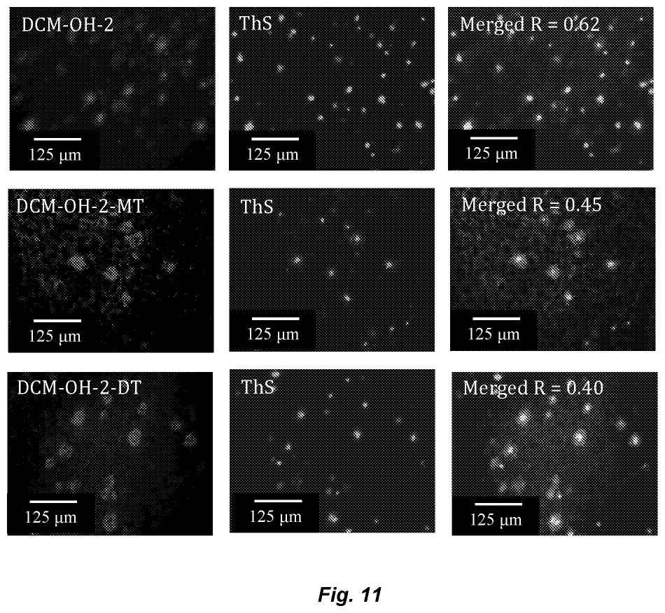

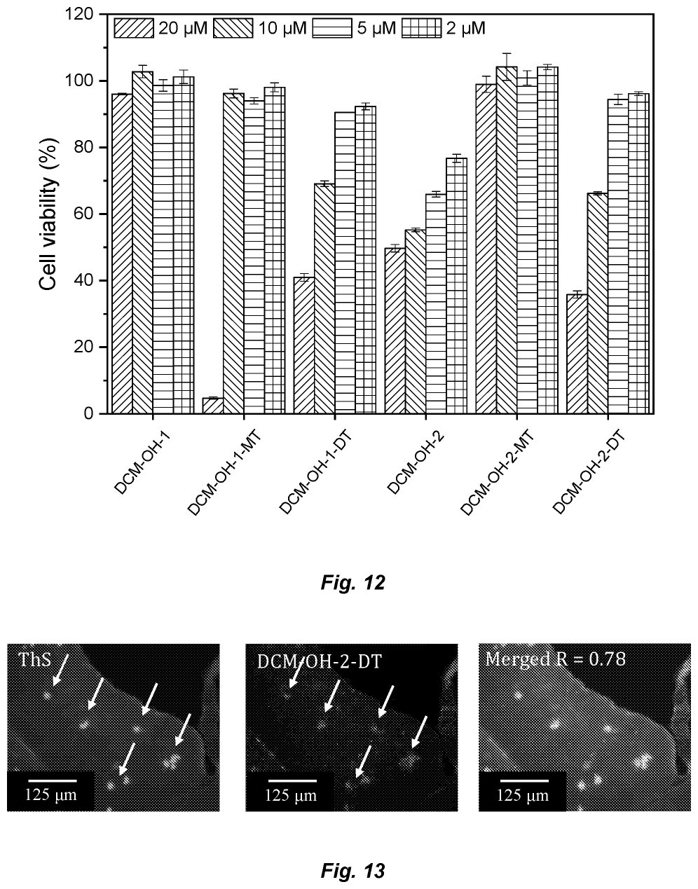

Fluorescence Imaging of 5×FAD Mouse Brain Sections and blood-brain barrier (BBB) permeability. To confirm the developed compounds can also bind to native Aβ aggregates, we performed fluorescence imaging studies and colocalization experiments on 5×FAD mouse brain sections. As shown in FIG. 10, DCM-OH-1 series of compounds colocalize well with anti-Ap antibody HJ 3.4, and DCM-OH-2 series of compounds colocalize well with ThS (FIG. 11). Interestingly, ThS binds to the core of Aβ aggregates, which represent the Aβ plaques/fibrils. While for the amphiphilic compounds, such as DCM-OH-2-MT and DCM-OH-2-DT, they bind to the peripheral region of the Aβ aggregates, which represent the less aggregated Aβ oligomers. This further supports that the compounds are selective and can bind to native soluble Aβ oligomers. Before investigating the blood-brain barrier (BBB) permeability of the developed compounds, their neurotoxicity was measured first (FIG. 12). These compounds are generally not toxic towards SH-SY5Y cells, except for DCM-OH-2. DCM-OH-2 leads to less than 80% cell viability even at 2 μM, which is probably due to its poor solubility. Nevertheless, the DCM-OH-2-DT was chosen as the candidate to test the blood-brain barrier (BBB) permeability. 12-month-old AD mice were treated with compound DCM-OH-2-DT (0.1 mg/Kg) for 10 days via intraperitoneal injection. At the end of treatment, mice brains were harvested and colocalization experiments were performed on the mice brain sections. As seen in FIG. 13, DCM-OH-2-DT exhibits good colocalization with ThS, indicating that the compound can successfully cross the BBB and bind the native Aβ aggregates.

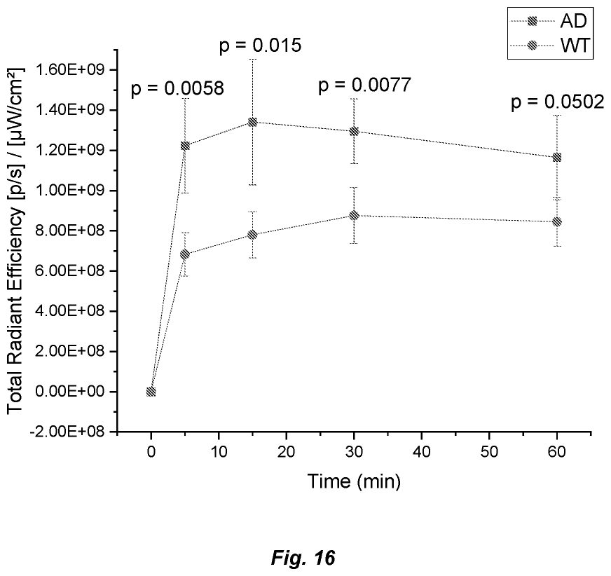

DCM-OH-2-DT was chosen as the best candidate for real-time in vivo fluorescence imaging studies. Seven-month-old AD mice and age-matched wild type (WT) mice were injected with the compound at 5 mg/kg. The NIR fluorescence intensities from the brain region were collected and analyzed over time. The signal from DCM-OH-2-DT increased to the maximum about 15 min after the injection, followed by a slow clearance of the compound. When comparing the signal intensity with WT mice, excitingly, DCM-OH-2-DT exhibited significantly higher fluorescence signal in AD mice at 5-, 15-, and 30-min post injection (FIG. 16). These imaging results further support the potential in vivo application of the developed compounds. Additionally, DCM-OH-2-DT can differentiate the AD mice vs. WT mice at a relative early age (7 months old), suggesting its potential application as an early diagnostic agent for AD.

In summary, the developed compounds can bind to soluble Aβ oligomers in vitro and on the mice brain sections. Moreover, the compounds can also cross the BBB and have the potential to be used as early diagnostic agents for AD.

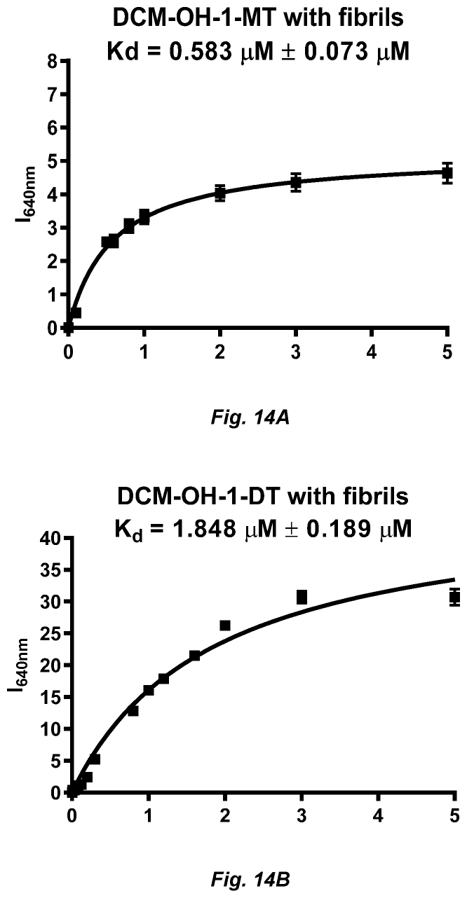

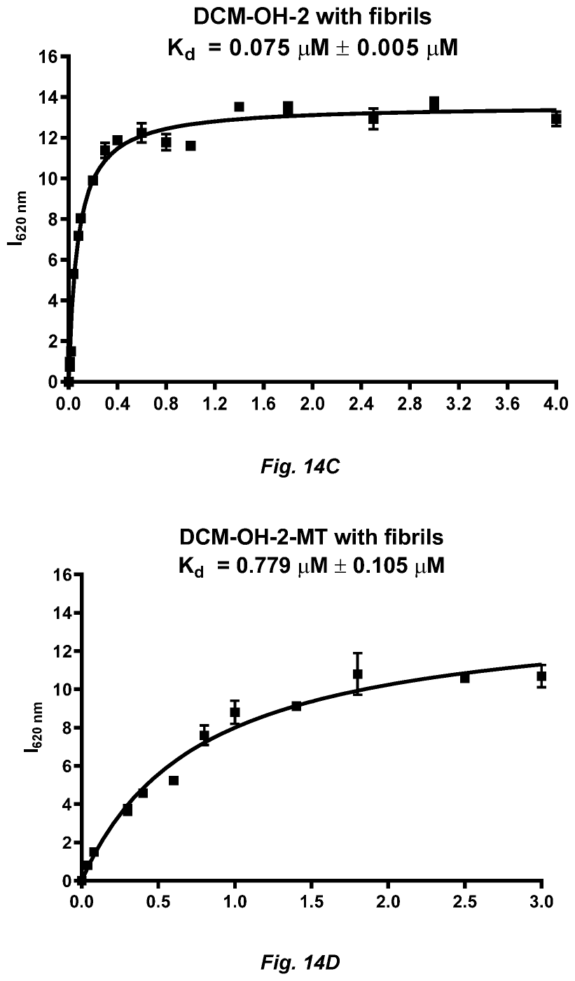

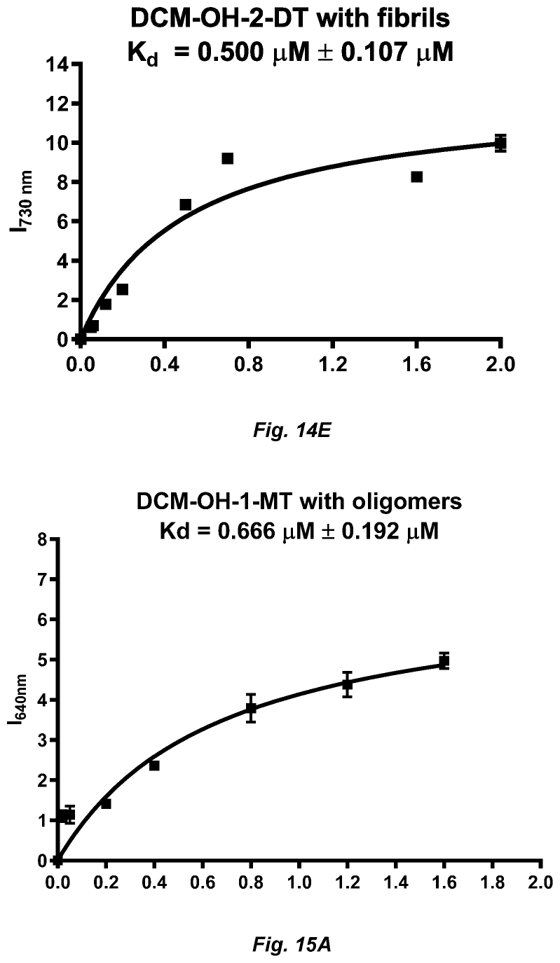

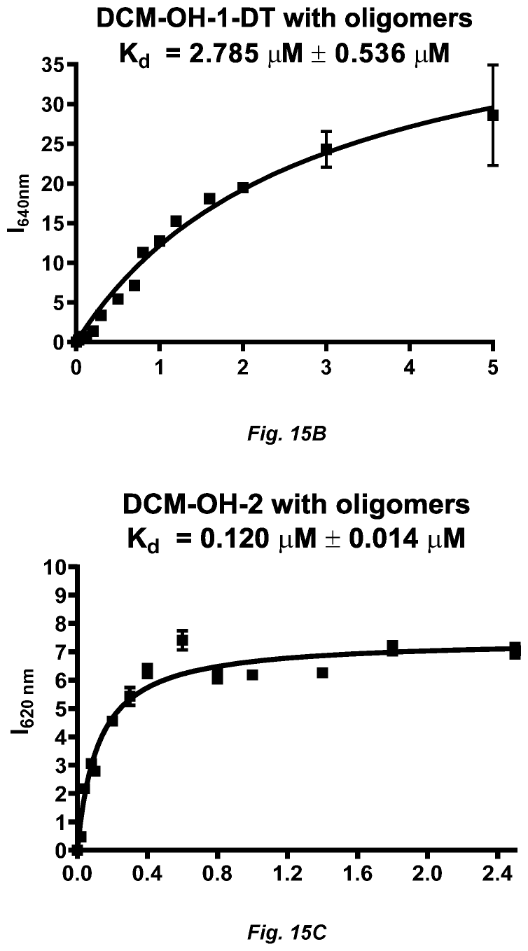

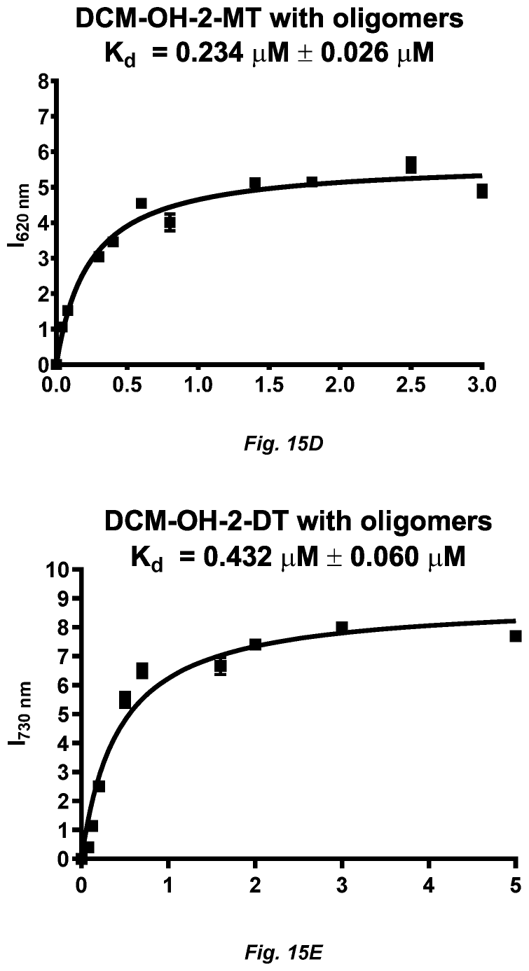

TABLE 3
Summary of K_{d} values of DCM compounds binding to
Aβ42 fibrils and oligomers (FIG. 14 and FIG. 15).

| | | Oligomers |
|---|---|---|
| Compounds | Fibrils (μM) | (μM) |
| DCM-OH-1 | N.A. | N.A. |
| DCM-OH-1-MT | 0.58 ± 0.07 | 0.67 ± 0.19 |
| DCM-OH-1-DT | 1.85 ± 0.19 | 2.88 ± 0.54 |
| DCM-OH-2 | 0.08 ± 0.01 | 0.12 ± 0.01 |
| DCM-OH-2-MT | 0.78 ± 0.11 | 0.23 ± 0.03 |
| DCM-OH-2-DT | 0.50 ± 0.11 | 0.43 ± 0.06 |

TABLE 4
Summary of Log D values of DCM compounds.

| Compounds | Log D |
|---|---|
| DCM-OH-1 | 1.00 ± 0.04 |
| DCM-OH-1-MT | 1.65 ± 0.06 |
| DCM-OH-1-DT | 1.51 ± 0.018 |
| DCM-OH-2 | 0.96 ± 0.07 |
| DCM-OH-2-MT | 1.13 ± 0.16 |
| DCM-OH-2-DT | 1.20 ± 0.07 |

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds or complexes described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating Alzheimer's disease in a mammal, which involve administering to a mammal having Alzheimer's disease an effective amount of a compound or composition described herein. A mammal includes a primate, human, rodent, canine, feline, bovine, ovine, equine, swine, caprine, bovine and the like.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. General Methods and Experimental Details

General methods. All reagents and solvents were purchased from commercial sources and used without further purification unless otherwise stated. 1,4-dimethyl-1,4,7-triazacyclononane (Me₂HTACN) was synthesized according to known procedures. HRMS data were obtained on a high-resolution electrospray ionization mass spectrometry (HR-ESI-MS, Thermo Scientific™ LTQ Orbitrap XL™ Hybrid Ion Trap-Orbitrap) (Thermo Scientific, USA). UV-visible spectra were recorded on a Varian Cary 50 Bio spectrophotometer, and fluorescence emission spectra were measured by a SpectraMax M2e plate reader (Molecular Devices, USA). 5×FAD transgenic mice overexpressing mutant human APP (695) with the Swedish (K670N, M671L), Florida (1716V), and London (V717I) were purchased from Jackson Laboratories (USA). Monoclonal anti-Ap-antibody (HJ 3.4) was obtained from Dr. Holtzman lab in the department of neurology at Washington University. The antibody was directly labeled with CF™ 594 dye using Mix-n-Stain™ CF™ 594 Antibody Labeling Kit purchased from Millipore Sigma (USA). Fluorescence images for brain sections were visualized using an Invitrogen EVOS FL Auto 2 Imaging System (Thermo Fisher, USA). Colocalization analysis and determination of the Pearson's correlation coefficient was performed with the imaging software Fiji. Mouse neuroblastoma N2a cell line and human neuroblastoma SH-SY5Y cell line were purchased from Millipore Sigma (USA). Confocal cell imaging studies were performed on a Zeiss LSM 880 confocal microscope, cell images were analyzed with Fiji and Zeiss Zen lite software. Compound purification was performed on a Teledyne Isco Combiflash Rf+. ¹H and ¹³C NMR spectra were recorded on Varian 400, Varian 500, or Carver B500 spectrometers. Spectra were analyzed and visualized with MestReNova (15.0). All other data analysis was performed using GraphPad Prism (8.0) or Origin 2020.

### Experimental Details

Preparation of the Aβ₄₂ Fibrils and Aβ₄₂ Oligomers. Aβ₄₂ fibrils: 1 mg of commercial Aβ₄₂ monomer powder (from GL biochem) was dissolved in 1 mL 1,1,1,3,3,3-hexafluoro-2-propanol (HFIP) at room temperature and incubated for 1 h. The resulting solution was divided equally into two Eppendorf tubes. The solution in each Eppendorf tube containing 0.5 mg Aβ₄₂ monomer was then evaporated overnight and dried by vacuum centrifuge to generate monomeric films. 10 μM Aβ₄₂ fibrils solution was prepared by dissolving the 0.5 mg monomeric films in 1.1 mL PBS buffer (with final DMSO concentration less than 1%) and stirring at 37° C. for 3 days.

Aβ₄₂ oligomers: The 0.5 mg monomeric films were dissolved in 1.1 mL PBS buffer (with final DMSO concentration less than 1%) and incubated at 4° C. overnight. The resulting solution was then centrifuged at 3000 rpm for 15 mins to remove the insoluble aggregates. The clear supernatant was then used for the following experiments. Aβ₄₂ oligomers concentration was checked by measuring the absorbance of the supernatant at 280 nm.

Fluorescence Spectral Testing of Compounds with Aps2 Fibrils and Aβ₄₂ Oligomers. The formation of Aβ₄₂ fibrils and oligomers was confirmed by the ThT fluorescence turn-on assay. The fluorescence spectra of ThT in PBS (100 μL PBS, 10.0 μM) were recorded as the baseline. After that, either Aβ₄₂ fibrils or oligomers were added to ThT in PBS. The final volume of the solution mixture should be 100 μL, in which the final concentrations of ThT and Aβ₄₂ species were10. μM and 25 μM, respectively.

For the interactions between Aps and compounds, the fluorescence spectra of the compounds' solution (100 μL PBS, 5.0 μM) were recorded as the baseline without adding various Aβ₄₂ species.

After that, either Aβ₄₂ fibrils or oligomers were added to the compounds' solutions in PBS. The final volume of the solution mixture should be 100 μL, in which the final concentrations of the compound and Aβ₄₂ species were 5. μM and 25 μM, respectively.

In Vitro Saturation Binding Studies with Aβ₄₂ Fibrils and Aβ₄₂ Oligomers. To a solution of increasing concentrations of compounds, a fixed concentration of the generated aggregated Aβ₄₂ fibrils or Aβ₄₂ oligomers solutions (5 to 15 NM) was added to yield a total volume of 100 μL. Nonspecific binding was determined without compounds. The mixture was incubated for 15 min at room temperature. All fluorescence data were obtained on a SpectraMax M2e plate reader (Molecular Devices). The fluorescent intensity was measured at the corresponding emission wavelength of each compound, and the K_{d} binding curves were generated by GraphPad Prism 8.0 with one site-specific binding model. Equation: Y=Bₘₐₓ*X/(K_{d}+X).

Cytotoxicity Studies. Alamar Blue assay was chosen for the cytotoxicity studies. Mouse neuroblastoma Neuro2A (N2A) cells were grown with DMEM/10% FBS in a petri dish at 37° C. in a humidified atmosphere with 5% CO2. Then N2A cells were seeded in a 96-well plate (1.0×10⁴/well). After 24 h incubation, cell media was changed to DMEM/N-2, and N2A cells were incubated for another 1 h. Then the cells were treated with different concentrations of compounds for 40 h, Alamar Blue solution (10 μL) was added, and the cells were incubated for another 90 min at 37° C. The fluorescence intensity of each well was measured at 590 nm (excitation wavelength=560 nm).

For the Cu²⁺-Ap-induced toxicity studies, cells treated were monomeric Aβ₄₂, Cu²⁺, monomeric Aβ₄₂+Cu²⁺, and monomeric Aβ₄₂+Cu²⁺+ compounds, respectively. After 40 h incubation, Alamar Blue solution (10 μL) was added, and the cells were incubated for another 90 min at 37° C. The fluorescence intensity of each well was measured at 590 nm (excitation wavelength=560 nm).

Histological Staining of 5×FAD Mice Brain Sections. 5×FAD transgenic mice brain sections were blocked with bovine serum albumin (2% BSA in PBS, pH 7.4, 10 min). Then the mice brain sections were transferred to a PBS solution of the compound and incubated for 1 h. After which, the brain sections were transferred to a PBS solution of Congo Red or antibody HJ 3.4 (Professor David Holtzman, 1 μg/ml) and incubated for another 1 h. Then, the brain sections were treated with BSA again (5 min) followed by washing with PBS (3×2 min), DI water (3×2 min). Finally, the mice brain sections were mounted with non-fluorescent mounting media, and the images were obtained by using EVOS FL Auto 2. ImageJ Fiji program was used for colocalization analysis and determination of the Pearson's correlation coefficient. The primary antibodies were labeled with dye CF594 via Mix-n-Stain™ CF™ 594 Antibody Labeling Kit (Sigma Aldrich).

Log D measurements. The Log D value was measured following a published procedure using slight modifications. In general, compounds in 0.5 mL octanol was subjected to partition with 0.5 mL octanol-saturated PBS. The whole mixture was stirred vigorously for 5 min, and centrifuged at 2,000 rpm for 5 min. The top octanol layer was separated for later fluorescence measurement. The remaining PBS layer was partitioned with 0.5 mL PBS-saturated octanol, and the second top octanol layer was then separated after vigorous stirring and centrifuging at 2,000 rpm for another 5 min. The two octanol layers' fluorescence spectra were recorded. The log D value was calculated by the fluorescence intensity ratio at the compounds' emission wavelength for the above two octanol extractions.

Cell Imaging Procedures. For oligomers and fibrils imaging: Human neuroblastoma SH-SY5Y cells were grown with DMEM/10% FBS at 37° C. in a humidified atmosphere with 5% CO2, and an 8-well μ-slides (ibidi) chambered coverslip was seeded the SH-SY5Y cells. At about 80% confluency (24 h after seeding), the cells were treated with DMSO as control group, 5 μM oligomers/fibrils and 5 μM (oligomers/fibrils+ compounds), respectively. After 24 h incubation, the media was removed from each well. The cells were then fixed with 200 μL 3.7% formaldehyde for 15 min and blocked with 200 μL 3% BSA for 10 min. 3% BSA was then removed from the cells and replaced with 100 μL CF594-HJ 3.4 (1 μg/mL in 3% BSA). The cells were incubated with CF594-HJ 3.4 for 2 h at room temperature and were washed with PBS (5×100 μL). Before imaging, the cells in each well were stained with 100 μL NucBlue reagent (Thermo Fisher Scientific) for 15 min and mounted with non-fluorescent mounting media. 5-10 images of each well were taken, and the images were processed and analyzed by Fiji and Zeiss Zen lite software. Three individual replicates were conducted and subjected to the statistical analysis.

For monomers +Cu²⁺ imaging: SH-5Y5Y were seeded as mentioned above, and cells were treated with DMSO as control group, 5 μM monomers, 5 μM (monomers +CuCl₂) and 5 μM (monomers +CuCl₂+compounds) respectively. After 48 h incubation, cells were treated and imaged the same way as mentioned above.

In vivo Imaging Studies. The real-time in vivo fluorescence imaging studies were performed with IVIS imaging system. 7-month-old 5×FAD mice (n=3) and age-matched control B6SJLF1/J mice (n=3) were shaved at the brain region before background imaging. All mice were injected with DCM-OH-2-DT (5 mg/kg, 15% DMSO, 85% PBS) via jugular vein injection. The mice were kept on the imaging stage under anesthesia with 2.5% isoflurane gas in an oxygen flow (1.5 L/min) before the imaging and were woken up after the imaging. Fluorescence signals from the brain were recorded at 5-, 15-, 30-, 60-min after the injection of DCM-OH-2-DT with a filter set (excitation at 470 nm and emission at 740 nm). Imaging data was analyzed by Living Image software, and an ROI was drawn around the brain region. The data were analyzed by subtracting the background fluorescence intensity of each mouse. P values were calculated by Student's t test.

### Synthetic Details.

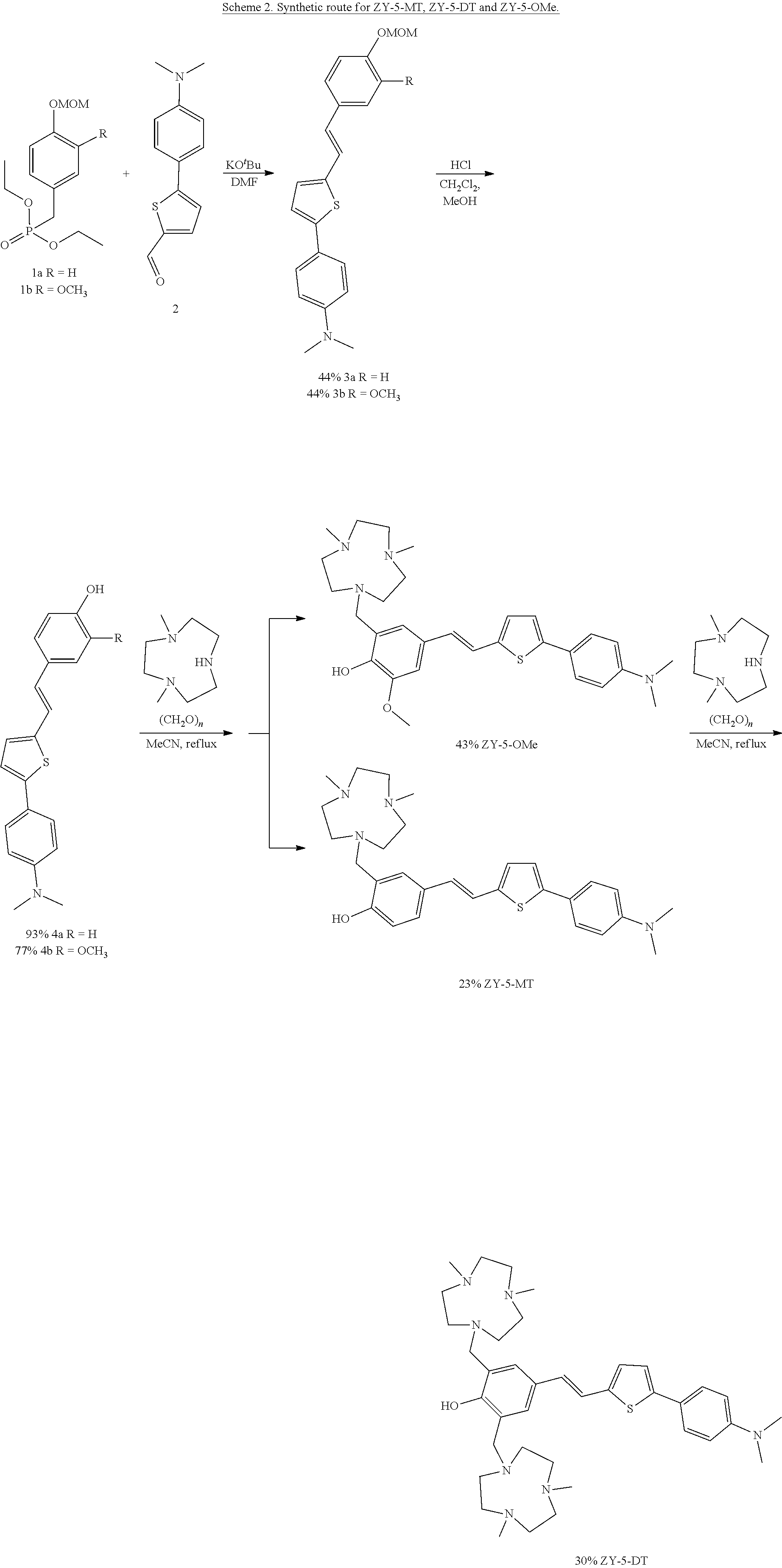

Compound 3a, 3b: Compound 1a (74 mg, 0.26 mmol, 1.5 eq) or 1b (70 mg, 0.22 mmol, 2.0 eq) was added to a solution of compound 2 (40 mg, 0.17 mmol or 25 mg, 0.11 mmol, 1.0 eq) in 5 mL DMF. Then potassium tert-butoxide (39 mg, 0.35 mmol, 2.0 eq or 30 mg, 0.27 mmol, 2.5 eq) was added to the solution slowly, and the solution color turned dark red. The whole reaction mixture was stirred at room temperature overnight. Then the mixture was diluted with dichloromethane, and the organic solution was washed with brine 3 times. The organic layer was dried over MgSO₄ and concentrated. The residue was purified by flash column chromatography on silica gel (eluent: hexane and ethyl acetate, 3:1). Compound 3a (28 mg) and 3b (19 mg) were both obtained in 44% isolated yield.

3a: ¹H NMR (400 MHz, CDCl₃) δ (ppm): 7.57-7.46 (m, 2H), 7.43-7.33 (m, 2H), 7.12-6.99 (m, 4H), 6.95 (d, J=3.7 Hz, 1H), 6.83 (d, J=16.2 Hz, 1H), 6.73 (d, J=8.4 Hz, 2H), 5.19 (s, 2H), 3.50 (s, 3H), 3.00 (s, 6H).

3b: ¹H NMR (500 MHz, CDCl₃): δ (ppm): 7.49 (d, J=8.8 Hz, 2H), 7.12 (d, J=8.3 Hz, 1H), 7.08 (d, J=16.0 Hz, 1H), 7.05 (d, J=3.6 Hz, 1H), 7.02 (d, J=2.0 Hz, 1H), 6.99 (dd, J=8.3, 2.0 Hz, 1H), 6.96 (d, J=3.7 Hz, 1H), 6.84 (s, 1H), 6.73 (d, J=8.4 Hz, 2H), 5.24 (s, 2H), 3.94 (s, 3H), 3.53 (s, 3H), 2.99 (s, 6H).

Compound 4a and 4b: Concentrated hydrogen chloride (2 mL) was added to a solution of 3a (28 mg, 0.077 mmol) or 3b (19 mg, 0.097 mmol) in dichloromethane (5 mL) and methanol (5 mL). The resulting mixture was stirred at room temperature for 12 h. The mixture was diluted with dichloromethane, and the organic solution was washed with a saturated sodium bicarbonate solution. The organic layer was dried over MgSO₄ and concentrated. The residue was purified by flash column chromatography on silica gel (eluent: hexane and ethyl acetate, 3:1). Compound 4a (23 mg, 0.072 mmol) and 4b (13 mg, 0.036 mmol) were obtained in 93% and 77% isolated yield, respectively. 4a: ¹H NMR (500 MHz, (CD3)₂CO) δ (ppm): 7.50 (d, J=8.6 Hz, 2H), 7.40 (d, J=9.0, 2H), 7.16 (d, J=16.1, 1H), 7.13 (d, J=3.6 Hz, 1H), 7.01 (d, J=3.7 Hz, 1H), 6.85 (m, 3H), 6.77 (d, J=8.8 Hz, 2H), 2.97 (d, J=1.5 Hz, 6H). ¹³C NMR (500 MHz, (CD3)₂CO) δ (ppm): 206.15, 157.45, 150.27, 143.12, 140.56, 128.64, 127.55, 127.01, 126.21, 122.41, 121.11, 119.27, 115.67, 112.50, 39.57.4b: ¹H NMR (400 MHz, CDCl₃) δ (ppm): 7.49 (d, J=8.3 Hz, 2H), 7.11-6.93 (m, 4H), 6.95 (d, J=3.8 Hz, 1H), 6.89 (d, J=8.6 Hz, 1H), 6.81 (d, J=16.0 Hz, 1H), 6.76-6.65 (d, J=8.3 Hz, 2H), 3.95 (s, 3H), 2.99 (s, 6H).

Compound ZY-5-MT and ZY-5-OMe: Paraformaldehyde (3 mg, 0.010 mmol) was added to a solution of 1,4-dimethyl-1,4,7-triazacyclononane (15 mg, 0.095 mmol) in MeCN (10 mL), and the mixture was refluxed for 30 min. Then a solution of compound 4a (23 mg, 0.072 mmol) or 4b (13 mg, 0.036 mmol) in MeCN (5 mL) was added to the reaction mixture, and the solution was further refluxed for another 16 h. The solvent was removed under vacuum, and the resulting residue was purified by C-18 reversed-phase column (eluent: MeCN/H₂O with 0.1% TFA, gradient wash from 10:90 to 30:70). The crude product collected from the reversed-phase column was then neutralized with saturated NaHCO₃ solution, extracted by dichloromethane (3×50 mL). The organic solvent was dried over MgSO₄ and concentrated to yield the final products ZY-5 (8.0 mg) and ZY-5-OMe (8.3 mg) in 23% and 43% isolated yield, respectively.

ZY-5-MT: ¹H NMR (500 MHz, CDCl₃) δ (ppm): 7.53-7.49 (m, 2H), 7.31 (dd, J=8.7, 2.2 Hz, 1H), 7.13 (d, J=2.2 Hz, 1H), 7.07-7.00 (m, 2H), 6.95-6.90 (m, 2H), 6.81 (d, J=16.0 Hz, 1H), 6.77-6.72 (m, 2H), 3.85 (s, 2H), 3.01 (s, 6H), 2.90 (dd, J=6.7, 3.6 Hz, 4H), 2.78-2.69 (m, 7H), 2.49 (s, 6H). ¹³C NMR (126 MHz, CDCl₃) δ (ppm): 157.93, 150.09, 143.34, 141.02, 128.28, 127.38, 127.30, 127.10, 126.71, 126.60, 123.58, 123.05, 121.30, 119.43, 117.06, 112.67, 60.33, 56.81, 52.77, 45.82, 40.62, 32.08, 29.85, 29.52, 22.84, 14.27. HRMS: calculated exact mass=491.2872 for [M+H]⁺, found 491.2849.

ZY-5-OMe: ¹H NMR (400 MHz, CD3CN) δ (ppm): 7.55-7.45 (m, 2H), 7.22 (dd, J=16.1, 0.7 Hz, 1H), 7.19-7.07 (m, 2H), 7.01 (d, J=3.8 Hz, 1H), 6.97 (d, J=2.0 Hz, 1H), 6.90-6.68 (m, 3H), 3.91 (s, 3H), 3.89 (s, 2H), 2.97 (s, 6H), 2.81 (m, 12H), 2.49 (s, 6H). HRMS: calculated exact mass=521.2972 for [M+H]⁺, found 521.2950.

Compound ZY-5-DT: Paraformaldehyde (5 mg, 0.167 mmol) was added to a solution of 1,4-dimethyl-1,4,7-triazacyclononane (20 mg, 0.127 mmol) in MeCN (10 mL), and the mixture was refluxed for 30 min. Then a solution of compound ZY-5-MT (23 mg, 0.047 mmol) in MeCN (5 mL) was added to the reaction mixture, which was refluxed for another 24 h. The solvent was removed under vacuum, and the resulting residue was purified by C-18 reversed-phase column (eluent: MeCN/H₂O with 0.1% TFA, gradient wash from 10:90 to 30:70). The crude product collected from the reversed-phase column was then neutralized with saturated NaHCO₃ solution, extracted by dichloromethane (3×50 mL). The organic solvent was dried over MgSO₄ and concentrated to yield the final product ZY-5-DT (9.3 mg) in 30% isolated yield.

ZY-5-DT: ¹H NMR (500 MHz, CDCl₃) δ (ppm): 7.53-7.48 (m, 2H), 7.22 (s, 2H), 7.07 (m, 2H), 6.97 (d, J=3.7 Hz, 1H), 6.80-6.71 (m, 3H), 3.96 (s, 4H), 3.02 (m, 14H), 2.97 (m, 8H), 2.91-2.83 (m, 8H), 2.57 (s, 12H). ¹³C NMR (126 MHz, CDCl₃) δ (ppm): 154.73, 149.03, 142.79, 139.19, 127.84, 127.54, 126.11, 125.58, 125.08, 123.46, 121.64, 120.19, 119.41, 111.47, 57.23, 53.58, 52.65, 51.01, 43.42, 39.43, 30.91, 28.68, 28.35, 21.68, 13.11. HRMS: calculated exact mass=660.4373 for [M+H]⁺, found 660.4402.

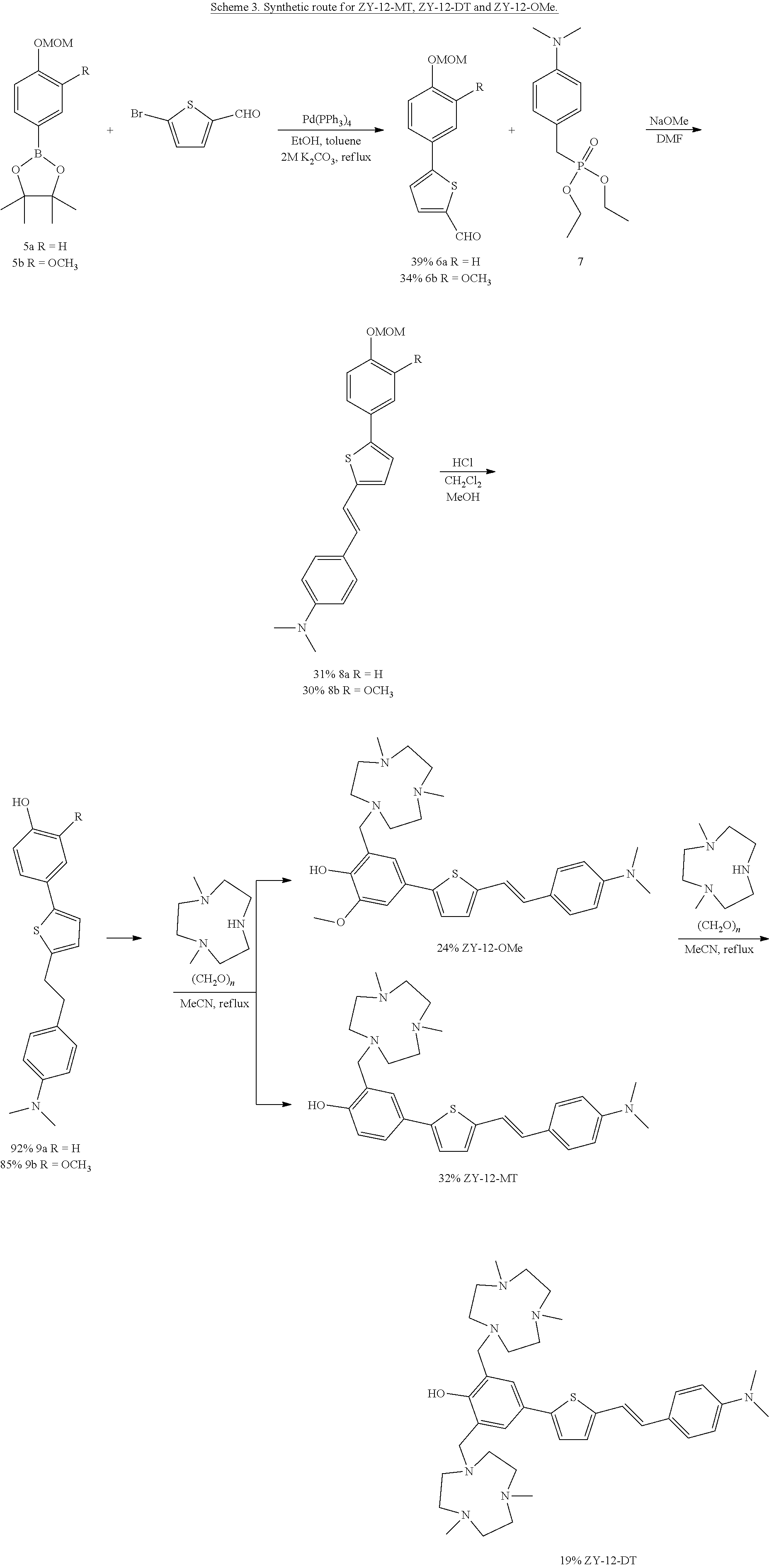

Compound 6a and 6b: (4-bromobenzaldehyde) (300 mg, 1.57 mmol, 1.3 eq) and 5a (327 mg, 1.24 mmol, 1 eq) or 5b (365 mg, 1.24 mmol, 1.0 eq) were dissolved in a mixture of 10 ml ethanol and 10 ml toluene. 2 ml of an aqueous K₂CO3 (2M) solution was added to the reaction mixture followed by the addition of Pd(PPh₃)₄ (70 mg, 0.062 mmol, 0.05 eq). Then the mixture was stirred and refluxed for 6 h under a nitrogen atmosphere. The solvent was removed under vacuum, and the residue was washed with brine and extracted with dichloromethane. Then the organic layer was dried over MgSO₄ and concentrated. The residue was then purified by flash column chromatography on silica gel (eluent: hexane and ethyl acetate, 5:1). Compounds 6a (120 mg) and 6b (117 mg) were obtained in 39% and 34% isolated yield, respectively.

6a: ¹H NMR (500 MHz, CDCl₃) δ (ppm): 9.89 (s, 1H), 7.74 (d, J=4.0, 1H), 7.63 (d, J=8.8 Hz, 2H), 7.33 (d, J=3.9, 1H), 7.12 (d, J=8.8 Hz, 2H), 5.25 (s, 2H), 3.52 (s, 3H), 1.27 (s, 3H).

6b: ¹H NMR (500 MHz, CDCl₃) δ (ppm): 9.89 (s, 1H), 7.74 (d, J=3.9, 1H), 7.34 (d, J=4.0, 1H), 7.25 (d, J=2.0 Hz, 1H), 7.22 (s, 1H), 7.18 (d, J=1.9 Hz, 1H), 5.29 (s, 2H), 3.97 (s, 3H), 3.55 (s, 3H). ¹³C NMR (126 MHz, CDCl₃) δ (ppm): 182.94, 154.62, 150.30, 148.04, 142.14, 137.78, 132.40, 128.82, 127.76, 123.76, 119.68, 116.69, 110.23, 95.58, 56.59, 56.30, 29.95.

Compound 8a and 8b: 6a (90 mg, 0.36 mmol, 1.0 eq) or 6b (100 mg, 0.36 mmol, 1.0 eq) was added to a solution of compound 7 (diethyl (4-(dimethylamino)benzyl)phosphonite) (100 mg, 0.36 mmol, 1.0 eq) in DMF (5 mL). Then sodium methoxide (59 mg, 1.09 mmol, 3.0 eq) was added to the reaction vessel in portions, and the reaction mixture was stirred at room temperature overnight. After which, dichloromethane was added to the reaction mixture, and the organic layer was washed with brine 3 times. The organic layer was then dried over MgSO₄ and concentrated. The residue was purified by flash column chromatography on silica gel (eluent: hexane and ethyl acetate, 3:1).

Compound 8a (41 mg, 0.11 mmol) and 8b (43 mg) were obtained in 31% and 30% yield, respectively.

8a: ¹H NMR (500 MHz, CDCl₃) δ (ppm): 7.55 (d, J=8.8 Hz, 2H), 7.40 (d, J=8.7 Hz, 2H), 7.12 (d, J=3.7 Hz, 1H), 7.08 (d, J=8.7 Hz, 2H), 7.03 (d, J=16.0 Hz, 2H), 6.95 (d, J=3.7 Hz, 1H), 6.89 (d, J=16.0 Hz, 1H), 6.74 (d, J=8.6 Hz, 4H), 5.23 (s, 2H), 3.53 (s, 4H), 3.02 (s, 6H).

8b: ¹H NMR (500 MHz, CDCl₃) δ (ppm): 7.45-7.37 (m, 2H), 7.23-7.15 (m, 3H), 7.14 (d, J=3.6 Hz, 1H), 7.04 (d, J=16.0 Hz, 1H), 6.96 (d, J=3.7 Hz, 1H), 6.90 (d, J=16.0 Hz, 1H), 6.79-6.71 (m, 2H), 5.40-5.27 (m, 2H), 3.98 (s, 3H), 3.57 (s, 3H), 3.19-2.83 (s, 6H).

Compound 9a and 9b: Concentrated hydrogen chloride (2 mL) was added to a solution of 8a (60 mg, 0.164 mmol) or 8b (42 mg, 0.106 mmol) in dichloromethane (5 mL) and methanol (5 mL). The resulting mixture was stirred at room temperature overnight. The solvent was then removed under vacuum, and dichloromethane was added to the residue. The organic solution was washed with NaHCO₃ solution and brine. The organic layer was dried over MgSO₄ and concentrated. The residue was then purified by flash column chromatography on silica gel (eluent: hexane and ethyl acetate, 1:1). Compound 9a (50 mg) and 9b (30 mg) were obtained in 92% and 85% isolated yield, respectively.

9a: ¹H NMR (500 MHz, (CD3)₂CO) δ (ppm): 7.51 (d, J=8.3 Hz, 2H), 7.41 (d, J=8.5, 2H), 7.17 (d, J=3.7 Hz, 1H), 7.12 (d, J=16.1 Hz, 1H), 6.99 (d, J=3.7 Hz, 1H), 6.92-6.83 (m, 3H), 6.74 (d, J=8.4 Hz, 2H), 2.97 (s, 6H).

9b: ¹H NMR (500 MHz, CDCl₃) δ (ppm): 7.40 (d, J=8.7 Hz, 2H), 7.17 (dd, J=8.2, 2.0 Hz, 1H), 7.12-7.08 (m, 2H), 7.04 (d, J=16.0 Hz, 1H), 6.98-6.93 (m, 2H), 6.88 (d, J=16.0 Hz, 1H), 6.74 (d, J=8.4 Hz, 2H), 3.99 (s, 3H), 3.02 (s, 6H).

Compound ZY-12-MT and ZY-12-OMe: Paraformaldehyde (5 mg, 0.167 mmol) was added to a solution of 1,4-dimethyl-1,4,7-triazacyclononane (20 mg, 0.127 mmol) in MeCN (10 mL), and the solution was refluxed for 30 min. Then a solution of compound 9a (33 mg, 0.103 mmol) or 9b (30 mg, 0.085 mmol) in MeCN (5 mL) was added to the reaction mixture, and it was further refluxed overnight. The solvent was then removed under vacuum, and the resulting residue was purified by C-18 reversed-phase column (eluent: MeCN/H₂O with 0.1% TFA, gradient wash from 10:90 to 30:70 and 50:50). The crude product collected from the reversed-phase column was then neutralized with saturated NaHCO₃ solution, extracted by dichloromethane (3×50 mL). The organic solvent was dried over MgSO₄ and concentrated to yield the final product. ZY-12-MT and ZY-12-OMe were obtained in 32% and 24% isolated yield, respectively.

ZY-12-MT: ¹H NMR (500 MHz, CDCl₃) δ (ppm): 7.34 (d, J=8.0 Hz, 1H), 7.29 (d, J=8.5 Hz, 2H), 7.17 (s, 1H), 7.09 (d, J=7.8 Hz, 1H), 6.97 (d, J=3.6 Hz, 1H), 6.92 (d, J=16.0 Hz, 1H), 6.83 (d, J=3.6 Hz, 1H), 6.75 (d, J=16.0 Hz, 1H), 6.63 (d, J=8.5 Hz, 2H), 3.79 (s, 2H), 2.92 (s, 6H), 2.80 (m, 4H), 2.67 (m, 8H), 2.46 (s, 6H). ¹³C NMR (126 MHz, CDCl₃) δ (ppm): 148.99, 141.05, 140.98, 128.85, 128.80, 126.94, 126.85, 126.31, 125.31, 124.86, 124.51, 124.48, 122.69, 120.84, 116.91, 116.10, 111.45, 111.07, 39.44, 30.91, 28.69, 28.35, 21.68, 13.11. HRMS: calculated exact mass=491.2845 for [M+H]⁺, found 491.2821.

ZY-12-OMe: ¹H NMR (500 MHz, CDCl₃) δ (ppm): 7.37 (d, J=8.6 Hz, 2H), 7.06 (d, J=3.7 Hz, 1H), 7.04 (d, J=2.0 Hz, 1H), 7.01 (d, J=16.0 Hz, 1H), 6.92 (d, J=3.7 Hz, 1H), 6.89 (d, J=2.0 Hz, 1H), 6.85 (d, J=16.0 Hz, 1H), 6.72 (d, J=8.7 Hz, 2H), 3.95 (s, 3H), 3.87 (s, 2H), 3.00 (m, 10H), 2.81-2.71 (m, 4H), 2.63 (m, 4H), 2.42 (s, 6H). ¹³C NMR (126 MHz, CDCl₃) δ (ppm): 148.99, 147.23, 146.58, 141.25, 141.07, 127.00, 126.31, 124.82, 124.43, 124.14, 122.22, 120.98, 117.20, 116.87, 111.44, 107.39, 59.69, 57.25, 56.92, 54.98, 52.26, 45.58, 39.42, 28.68. HRMS: calculated exact mass =521.2950 for [M+H]⁺, found 521.2943.

Compound ZY-12-DT: Paraformaldehyde (5 mg, 0.167 mmol) was added to a solution of 1,4-dimethyl-1,4,7-triazacyclononane (20 mg, 0.127 mmol) in MeCN (10 mL), and was heated under reflux for 30 min. Then a solution of compound 9a (33 mg, 0.103 mmol) or 9b (30 mg, 0.085 mmol) in MeCN (5 mL) was added to the reaction mixture, and it was further refluxed overnight. The solvent was removed under vacuum, and the resulting residue was purified by C-18 reversed-phase column (eluent: MeCN/H₂O with 0.1% TFA, gradient wash from 10:90 to 30:70 and 50:50). The crude product collected from the reversed-phase column was then neutralized with saturated NaHCO₃ solution, extracted by dichloromethane (3×50 mL). The organic solvent was dried over MgSO₄ and concentrated to yield the final product. ZY-12-DT were obtained in 19% isolated yield.

ZY-12-DT: ¹H NMR (500 MHz, CDCl₃) δ (ppm): 7.37 (d, J=8.8 Hz, 2H), 7.30 (s, 2H), 7.06 (d, J=3.7 Hz, 1H), 7.01 (d, J=16.1 Hz, 1H), 6.92 (d, J=3.7 Hz, 1H), 6.84 (d, J=16.0 Hz, 1H), 6.72 (d, J=8.9 Hz, 2H), 3.88 (s, 4H), 3.00 (s, 6H), 3.00-2.92 (m, 8H), 2.89-2.77 (m, 16H), 2.45 (s, 12H). ¹³C NMR (126 MHz, CDCl₃) δ (ppm): 157.18, 150.19, 142.44, 141.52, 130.05, 128.39, 127.49, 126.63, 126.00, 125.41, 125.25, 124.16, 122.17, 117.82, 112.56, 58.57, 56.09, 52.60, 45.38, 40.55, 32.04, 31.05, 29.78, 29.39, 22.81, 14.25. HRMS: calculated exact mass=660.4424 for [M+H]⁺, found 660.4427.

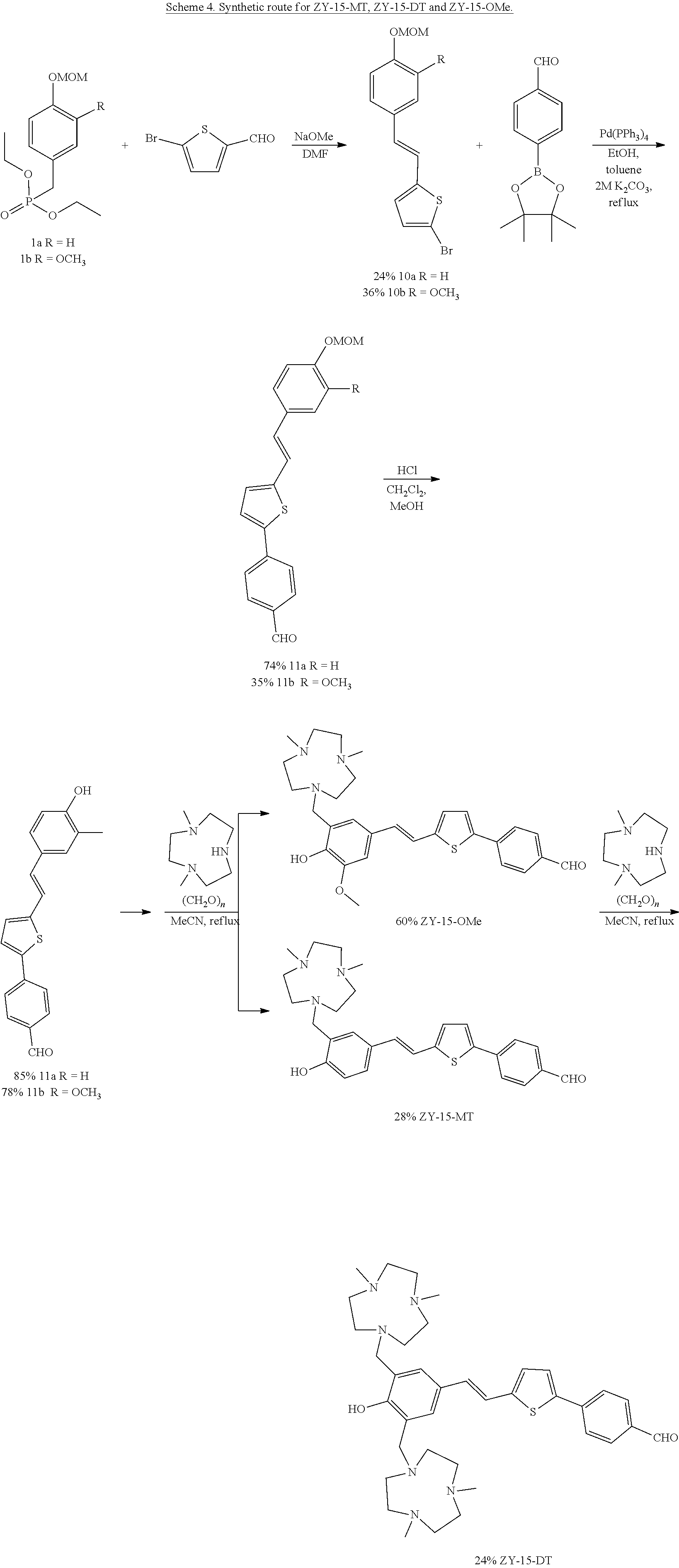

Compound 10a and 10b: 5-bromothiophene-2-carbaldehyde (265 mg, 1.39 mmol, 1.0 eq) was added to a solution of compound 1a (400 mg, 1.39 mmol, 1 eq) or 1b (440 mg, 1.39 mmol, 1.0 eq) in DMF (5 ml). Then sodium methoxide (234 mg, 2.10 mmol, 1.5 eq) was added to the reaction vessel in portions, and the reaction mixture was stirred at room temperature for 24 hr. After which, dichloromethane was added to the reaction mixture, and the organic layer was washed with brine 3 times. The organic layer was then dried over MgSO₄ and concentrated. The residue was purified by flash column chromatography on silica gel (eluent: hexane and ethyl acetate, 5:1). Compound 10a (108 mg) and 10b (178 mg) were obtained in 24% and 36% isolated yield, respectively.

10a: ¹H NMR (500 MHz, CDCl₃) δ (ppm): 7.38 (d, J=8.4 Hz, 2H), 7.07-6.96 (m, 3H), 6.95 (d, J=3.8 Hz, 1H), 6.81-6.74 (m, 2H), 5.20 (s, 2H), 3.50 (s, 3H), 2.18 (s, 3H). ¹³C NMR (500 MHz, CDCl₃) δ (ppm): 157.32, 145.06, 130.72, 128.55, 127.83, 125.97, 119.87, 116.73, 94.60, 56.31, 31.19.

10b: ¹H NMR (500 MHz, CDCl₃) δ (ppm): 7.08 (dd, J=8.4, 1.2 Hz, 1H), 7.02-6.92 (m, 3H), 6.91 (dd, J=3.9, 1.3 Hz, 1H), 6.78-6.69 (m, 2H), 5.21 (s, 2H), 3.90 (s, 3H), 3.49 (s, 3H).

Compound Ha and 11b: (4-(4,4,5,5-tetramethyl-1,3,2-dioxaborolan-2-yl)benzaldehyde) (71 mg, 0.31 mmol, 1.0 eq) and compound 10a (100 mg, 0.31 mmol, 1.0 eq) or 10b (70 mg, 0.30 mmol, 1.0 eq) was dissolved in a mixture of solvent of 10 ml ethanol and 10 ml toluene. 2 ml of an aqueous K₂CO3 (2M) solution was added to the reaction mixture followed by the addition of Pd(PPh₃)₄ (18 mg, 0.015 mmol, 0.05 eq). The mixture was then stirred overnight under reflux. The solvent was removed under vacuum, and the residue was washed with brine and extracted with dichloromethane. Then the organic layer was collected, dried over MgSO₄, and concentrated. The residue was purified by flash column chromatography on silica gel (eluent: hexane and ethyl acetate, 6:1). The compounds 11a (80 mg) and 11b (44 mg) were obtained in 74% and 35% isolated yield, respectively.

11a: ¹H NMR (400 MHz, CDCl₃) δ (ppm): 10.00 (s, 1H), 7.89 (d, J=8.4 Hz, 2H), 7.75 (d, J=8.4 Hz, 2H), 7.42 (d, J=8.7 Hz, 2H), 7.38 (d, J=3.8 Hz, 1H), 7.09 (d, J=16.1 Hz, 1H), 7.06-7.01 (m, 3H), 6.95 (d, J=16.1 Hz, 1H), 5.20 (s, 2H), 3.50 (s, 3H).

11b: ¹H NMR (400 MHz, CDCl₃) δ (ppm): 9.99 (s, 1H), 7.88 (d, J=8.2 Hz, 2H), 7.74 (d, J=8.3 Hz, 2H), 7.38 (d, J=3.8 Hz, 1H), 7.14 (d, J=8.1 Hz, 1H), 7.10 (d, J=16.0 Hz, 1H), 7.04 (m, 4H), 6.94 (d, J=16.0 Hz, 1H), 5.26 (s, 2H), 3.95 (s, 3H), 3.53 (s, 3H).

Compound 12a and 12b: Concentrated hydrogen chloride (2 mL) was added to a solution of 11a (30 mg, 0.086 mmol) or 11b (40 mg, 0.105 mmol) in a mixture of dichloromethane (5 mL) and methanol (5 mL). The resulting mixture was stirred at room temperature for 12 h. The solvent was removed under vacuum, and dichloromethane was added to the residue. The organic solution was washed with NaHCO₃ solution. The organic layer was dried over MgSO₄ and concentrated. The residue was then purified by flash column chromatography on silica gel (eluent: hexane and ethyl acetate, 3:1). Compound 12a (22 mg) and 12b (27 mg) were obtained in 85% and 78% isolated yield, respectively.

12a: ¹H NMR (400 MHz, (CD3)₂CO) δ (ppm): 10.02 (s, 1H), 7.94 (d, J=8.3 Hz, 2H), 7.89 (d, J=8.3 Hz, 2H), 7.59 (d, J=3.8 Hz, 1H), 7.45 (d, J=8.6 Hz, 3H), 7.23 (d, J=16.2 Hz, 1H), 7.16 (d, J=3.9 Hz, 1H), 7.01 (d, J=16.1 Hz, 1H), 6.85 (d, J=8.6 Hz, 2H).

12b: ¹H NMR (500 MHz, CDCl₃) δ (ppm): 10.02 (s, 1H), 7.93-7.88 (m, 2H), 7.79-7.74 (m, 2H), 7.40 (d, J=3.7 Hz, 1H), 7.11-7.06 (m, 2H), 7.03 (m, 2H), 6.98-6.91 (m, 2H), 3.98 (s, 3H). ¹³C NMR (126 MHz, CDCl₃) δ (ppm): 191.42, 146.80, 145.99, 144.93, 140.41, 140.01, 134.93, 130.52, 129.58, 129.29, 126.87, 125.75, 125.60, 120.60, 119.35, 114.73, 108.21, 55.96.

Compound ZY-15-MT and ZY-15-OMe: Paraformaldehyde (3 mg, 0.100 mmol) was added to a solution of 1,4-dimethyl-1,4,7-triazacyclononane (10 mg, 0.063 mmol) in MeCN (10 mL), and the solution was refluxed for 30 min. Then a solution of compound 12a (22 mg, 0.071 mmol) or 12b (20 mg, 0.059 mmol) in MeCN (5 mL) was added to the reaction mixture, and it was heated under reflux overnight. The solvent was removed under vacuum, and the resulting residue was purified by C-18 reversed-phase column (eluent: MeCN/H₂O with 0.1% TFA, gradient wash from 10:90 to 30:70 to 50:50). The crude product collected from the reversed-phase column was then neutralized with saturated NaHCO₃ solution, extracted by dichloromethane (3×50 mL). The organic solvent was dried over MgSO₄ and concentrated to yield the final products. Compounds ZY-15-MT (9 mg) and ZY-15-OMe (18 mg) were obtained in 28% and 60% isolated yield, respectively.

ZY-15-MT: ¹H NMR (500 MHz, CDCl₃) δ (ppm): 9.91 (s, 1H), 7.83-7.74 (d, J=8.3 Hz, 2H), 7.65 (d, J=8.2 Hz, 2H), 7.28 (d, J=3.8 Hz, 1H), 7.23 (dd, J=8.3, 2.2 Hz, 1H), 7.19 (s, 1H), 7.07 (d, J=2.2 Hz, 1H), 7.00-6.89 (m, 2H), 6.82 (d, J=16.0 Hz, 1H), 3.78 (s, 2H), 2.80 (m, 4H), 2.64 (m, 8H), 2.44 (s, 6H). ¹³C NMR (126 MHz, CDCl₃) δ (ppm): 190.35, 144.26, 139.02, 133.82, 129.45, 128.52, 126.52, 126.43, 125.53, 124.72, 124.50, 122.72, 117.49, 116.06, 58.26, 54.64, 50.40, 44.06, 30.91, 28.68, 28.35, 21.68, 13.11. HRMS: calculated exact mass=476.2372 for [M+H]⁺, found 476.2369.

ZY-15-OMe: ¹H NMR (500 MHz, CDCl₃) δ (ppm): 10.01 (s, 1H), 7.90 (d, J=8.3 Hz, 2H), 7.76 (d, J=8.2 Hz, 2H), 7.39 (d, J=3.8 Hz, 1H), 7.11-7.04 (m, 2H), 6.99 (d, J=2.0 Hz, 1H), 6.95-6.81 (m, 2H), 3.96 (s, 3H), 3.87 (s, 2H), 2.96 (m, 4H), 2.91-2.77 (m, 8H), 2.51 (s, 6H). ¹³C NMR (126 MHz, CDCl₃) δ (ppm): 190.35, 147.08, 143.95, 139.30, 138.97, 133.89, 129.47, 128.50, 126.93, 126.72, 125.77, 124.74, 124.55, 122.29, 119.90, 118.06, 107.43, 62.60, 55.06, 51.75, 44.29, 30.89, 29.91, 28.69, 28.35, 21.68, 13.11. HRMS: calculated exact mass=506.2477 for [M+H]⁺, found 506.2464.

Compound ZY-15-DT: Paraformaldehyde (5 mg, 0.167 mmol) was added to a solution of 1,4-dimethyl-1,4,7-triazacyclononane (20 mg, 0.126 mmol) in MeCN (10 mL), and the mixture was heated under reflux for 30 min. Then a solution of compound ZY-15-MT (33 mg, 0.069 mmol) in MeCN (5 mL) was added to the reaction mixture, and the solution mixture was refluxed for another 24 h. The solvent was removed under vacuum, and the resulting residue was purified by C-18 reversed-phase column (eluent: MeCN/H₂O with 0.1% TFA, gradient wash from 10:90 to 30:70). The crude product collected from the reversed-phase column was then neutralized with saturated NaHCO₃ solution, extracted by dichloromethane (3×50 mL). The organic solvent was dried over MgSO₄ and concentrated to yield ZY-15-DT (11 mg) in 24% isolated yield.

ZY-15-DT: ¹H NMR (500 MHz, CDCl₃) δ (ppm): 9.92 (s, 1H), 7.81 (d, J=8.1 Hz, 2H), 7.67 (d, J=8.1 Hz, 2H), 7.30 (d, J=3.7 Hz, 1H), 7.19 (s, 1H), 7.15 (s, 1H), 7.03-6.93 (m, 2H), 6.82 (d, J=16.0 Hz, 1H), 3.78 (s, 4H), 2.89 (m, 8H), 2.69 (m, 16H), 2.34 (s, 12H). ¹³C NMR (126 MHz, CDCl₃) δ (ppm): 190.35, 156.69, 144.12, 139.01, 133.85, 129.47, 128.37, 126.40, 125.98, 125.61, 124.74, 124.54, 123.05, 117.68, 57.50, 55.65, 52.39, 44.82, 30.91, 28.68, 28.30, 21.71, 13.11. HRMS: calculated exact mass=645.3951 for [M+H]⁺, found 645.3931.

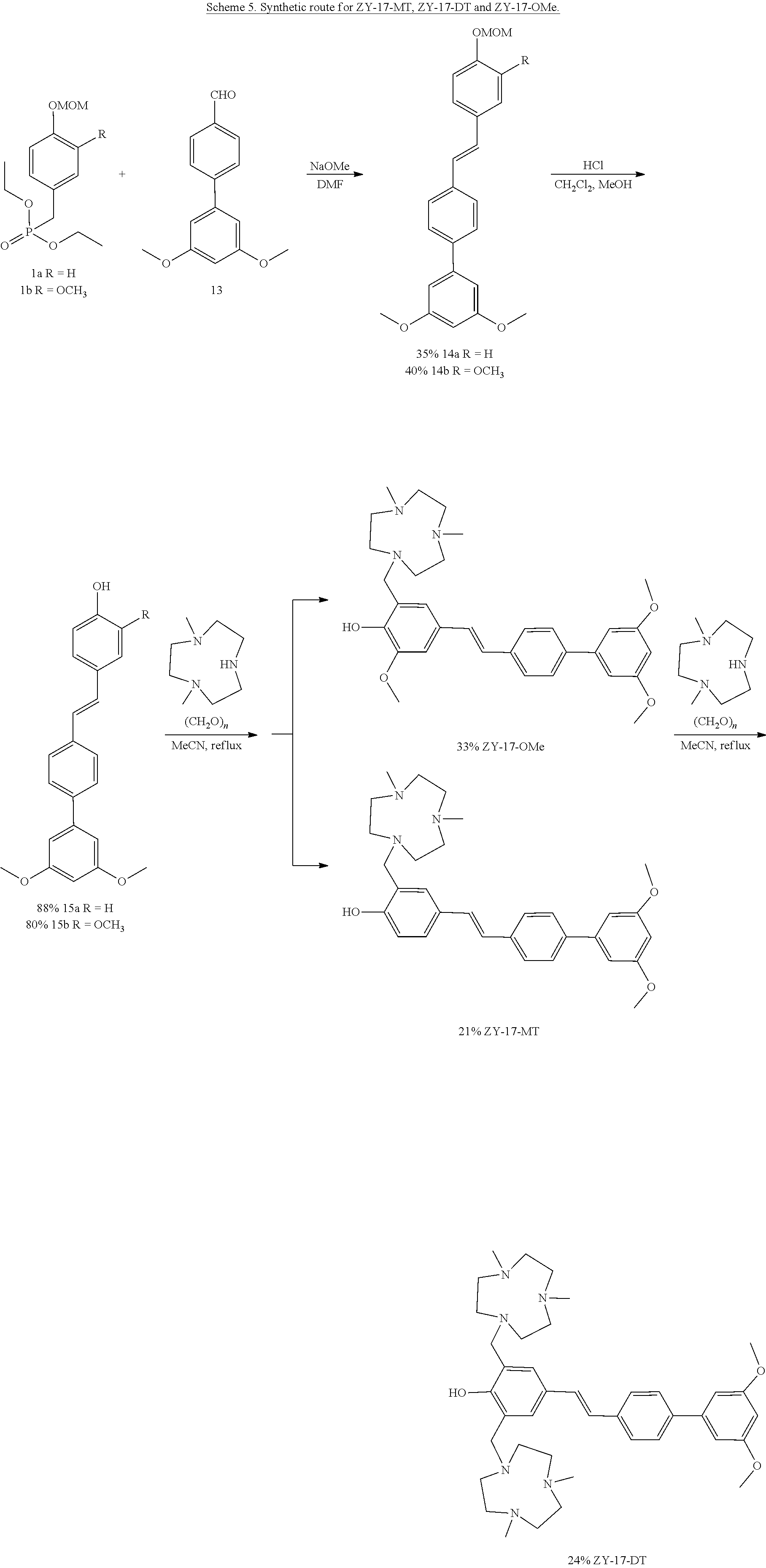

Compound 14a and 14b: Compound 1a (59 mg, 0.206 mmol, 1.0 eq) or 1b (65 mg, 0.206 mmol, 1.0 eq) was added to a solution of compound 13 (50 mg, 0.206 mmol, 1.0 eq) in DMF (5 mL). Then sodium methoxide (33 mg, 0.619 mmol, 3.0 eq) was added to the solution in portions, and the reaction mixture was stirred at room temperature overnight. Then dichloromethane was added to the mixture, and the organic solution was washed with brine 3 times. The organic layer was then dried over MgSO₄ and concentrated. The residue was purified by flash column chromatography on silica gel (eluent: hexane and ethyl acetate, 5:1). Compound 14a (27 mg) and 14b (34 mg) were obtained in 35% and 40% isolated yield, respectively.

14a: ¹H NMR (500 MHz, CDCl₃) δ (ppm): 7.53-7.44 (m, 4H), 7.42-7.36 (d, J=8.6 Hz, 2H), 7.03 (d, J=16.4 Hz, 1H), 6.99-6.89 (m, 3H), 6.68 (d, J=2.3 Hz, 2H), 6.39 (t, J=2.3 Hz, 1H), 5.12 (s, 2H), 3.78 (s, 5H), 3.42 (s, 3H). ¹³C NMR (126 MHz, CDCl₃) δ (ppm): 160.06, 155.91, 141.93, 138.92, 135.92, 130.23, 127.26, 126.69, 126.35, 125.62, 125.50, 115.44, 104.18, 98.26, 93.39, 55.01, 54.40. 14b: ¹H NMR (500 MHz, CDCl₃) δ (ppm): 7.66-7.53 (m, 4H), 7.18 (d, J=8.2 Hz, 1H), 7.15-7.02 (m, 4H), 6.83-6.77 (s, 2H), 6.51 (s, 1H), 5.29 (s, 2H), 3.99 (s, 3H), 3.88 (s, 6H), 3.57 (s, 3H). ¹³C NMR (126 MHz, CDCl₃) δ (ppm): 161.38, 150.16, 146.67, 143.17, 140.30, 137.08, 132.29, 128.80, 127.65, 127.08, 126.96, 120.10, 116.66, 109.67, 105.47, 99.57, 95.74, 56.51, 56.19, 55.66.

Compound 15a, 15b: Concentrated hydrogen chloride (2 mL) was added to a solution of 14a (50 mg, 0.133 mmol) or 14b (65 mg, 0.160 mmol) in a mixture of dichloromethane (5 mL) and methanol (5 mL). The resulting mixture was stirred at room temperature overnight. The solvent was then removed under vacuum, and dichloromethane was added to the residue. The organic solution was washed with NaHCO₃ solution and brine. The organic layer was dried over MgSO₄ and concentrated. The residue was then purified by flash column chromatography on silica gel (eluent: hexane and ethyl acetate, 3:1). Compound 15a (39 mg) and 15b (46 mg) were obtained in 88% and 80% isolated yield, respectively.

15a: ¹H NMR (500 MHz, CDCl₃) δ (ppm): 7.65-7.54 (m, 4H), 7.46 (dd, J=8.0, 4.0 Hz, 2H), 7.13 (d, J=16.2 Hz, 1H), 7.03 (d, J=16.3 Hz, 1H), 6.88 (d, J=8.5 Hz, 2H), 6.79 (d, J=2.3 Hz, 2H), 6.51 (t, J=2.3 Hz, 1H), 3.89 (s, 6H). ¹³C NMR (126 MHz, CDCl₃) δ (ppm): 161.34, 155.63, 143.24, 140.13, 137.26, 130.56, 128.59, 128.23, 127.64, 126.86, 126.36, 115.93, 105.50, 99.55, 55.70.

15b: ¹H NMR (126 MHz, CDCl₃) δ (ppm): 7.59 (q, J=8.4 Hz, 4H), 7.15-7.06 (m, 3H), 7.02 (d, J=16.2 Hz, 1H), 6.96 (d, J=7.9 Hz, 1H), 6.80 (d, J=2.3 Hz, 2H), 6.51 (t, J=2.3 Hz, 1H), 3.98 (s, 3H), 3.89 (s, 6H). ¹³C NMR (126 MHz, CDCl₃) δ (ppm): 160.05, 145.72, 144.65, 141.90, 138.81, 135.92, 128.93, 127.76, 126.34, 125.54, 124.89, 119.52, 113.58, 107.23, 104.16, 98.24, 54.88, 54.39.

Compound ZY-17-MT and ZY-17-OMe: Paraformaldehyde (5 mg, 0.167 mmol) was added to a solution of 1,4-dimethyl-1,4,7-triazacyclononane (20 mg, 0.126 mmol) in MeCN (10 mL), and the solution mixture was heated under reflux for 30 min. Then a solution of compound 15a (30 mg, 0.09 mmol) or 15b (40 mg, 0.11 mmol) in MeCN (5 mL) was added to the reaction mixture, and it was refluxed overnight. The solvent was removed under vacuum, and the resulting residue was purified by C-18 reversed-phase column (eluent: MeCN/H₂O with 0.1% TFA, gradient wash from 10:90 to 30:70 to 50:50). The crude product collected from the reversed-phase column was then neutralized with saturated NaHCO₃ solution, extracted by dichloromethane (3×50 mL). The organic solvent was dried over MgSO₄ and concentrated to yield the final product. Compound ZY-17-MT (9 mg) and ZY-17-OMe (19 mg) were obtained in 21% and 33% isolated yield, respectively.

ZY-17-MT: ¹H NMR (500 MHz, CDCl₃) δ (ppm): 7.47 (q, J=8.3 Hz, 4H), 7.33-7.25 (m, 1H), 7.15-7.05 (m, 2H), 7.01-6.94 (m, 1H), 6.89 (d, J=16.3 Hz, 1H), 6.68 (d, J=2.3 Hz, 2H), 6.39 (t, J=2.2 Hz, 1H), 3.79 (s, 6H), 3.58 (s, 2H), 2.85 (m, 8H), 2.69 (m, 4H), 2.58 (s, 6H). ¹³C NMR (126 MHz, CDCl₃) δ (ppm): 161.21, 158.54, 143.17, 139.72, 137.44, 128.97, 128.29, 127.48, 127.33, 126.59, 125.11, 123.67, 116.96, 105.31, 99.38, 60.63, 57.57, 55.57, 53.04, 46.31. HRMS: calculated exact mass=502.3070 for [M+H]⁺, found 502.3051.

ZY-17-OMe: ¹H NMR (500 MHz, CDCl₃) δ (ppm): 7.57 (q, J=8.4 Hz, 4H), 7.11-7.04 (m, 2H), 6.99 (d, J=16.2 Hz, 1H), 6.90 (d, J=1.9 Hz, 1H), 6.77 (d, J=2.3 Hz, 2H), 6.49 (t, J=2.3 Hz, 1H), 3.96 (s, 3H), 3.88 (s, 6H), 3.00 (m, 4H), 2.90 (m, 8H), 2.57 (s, 6H). ¹³C NMR (126 MHz, CDCl₃) δ (ppm): 161.11, 147.94, 142.89, 139.91, 136.87, 128.72, 127.41, 126.57, 125.95, 123.48, 121.52, 108.65, 105.20, 98.94, 61.64, 58.27, 56.14, 55.45, 54.72, 53.73, 51.50, 44.19, 31.94, 30.94, 29.67, 22.70, 14.14. HRMS: calculated exact mass=532.3175 for [M+H]⁺, found 532.3163.

Compound ZY-17-DT: Paraformaldehyde (5 mg, 0.167 mmol) was added to a solution of 1,4-dimethyl-1,4,7-triazacyclononane (20 mg, 0.126 mmol) in MeCN (10 mL), and the mixture was heated under reflux for 30 min. Then a solution of compound ZY-17-MT (23 mg, 0.046 mmol) in MeCN (5 mL) was added to the reaction mixture, and it was refluxed for another 24 h. The solvent was removed under vacuum, and the resulting residue was purified by C-18 reversed-phase column (eluent: MeCN/H₂O with 0.1% TFA, gradient wash from 10:90 to 30:70). The crude product collected from the reversed-phase column was then neutralized with saturated NaHCO₃ solution, extracted by dichloromethane (3×50 mL). The organic solvent was dried over MgSO₄ and concentrated to yield ZY-17-DT (7 mg) in 24% isolated yield.

ZY-17-DT: ¹H NMR (500 MHz, CDCl₃) δ (ppm): 7.47 (q, J=8.3 Hz, 4H), 7.19 (s, 2H), 6.98 (d, J=16.2 Hz, 1H), 6.90 (d, J=16.3 Hz, 1H), 6.68 (d, J=2.3 Hz, 2H), 6.39 (t, J=2.3 Hz, 1H), 3.77 (m, 10H), 2.87 (m, 8H), 2.67 (m, 16H), 2.32 (s, 12H). ¹³C NMR (126 MHz, CDCl₃) δ (ppm): 160.06, 156.24, 141.96, 138.64, 136.17, 128.19, 127.68, 126.61, 126.35, 126.25, 125.72, 125.44, 124.15, 123.19, 104.15, 98.23, 57.54, 55.82, 54.41, 52.58, 44.98, 28.68. HRMS: calculated exact mass=671.4649 for [M+H]⁺, found 671.4651.

### Example 2. Synthesis Of DCM Compounds

All reagents and solvents were purchased from commercial sources and used without further purification unless otherwise stated. 1,4-dimethyl-1,4,7-triazacyclononane (Me2HTACN) was synthesized according to our reported procedures (Inorg Chem 2017, 56 (22), 13801-13814).

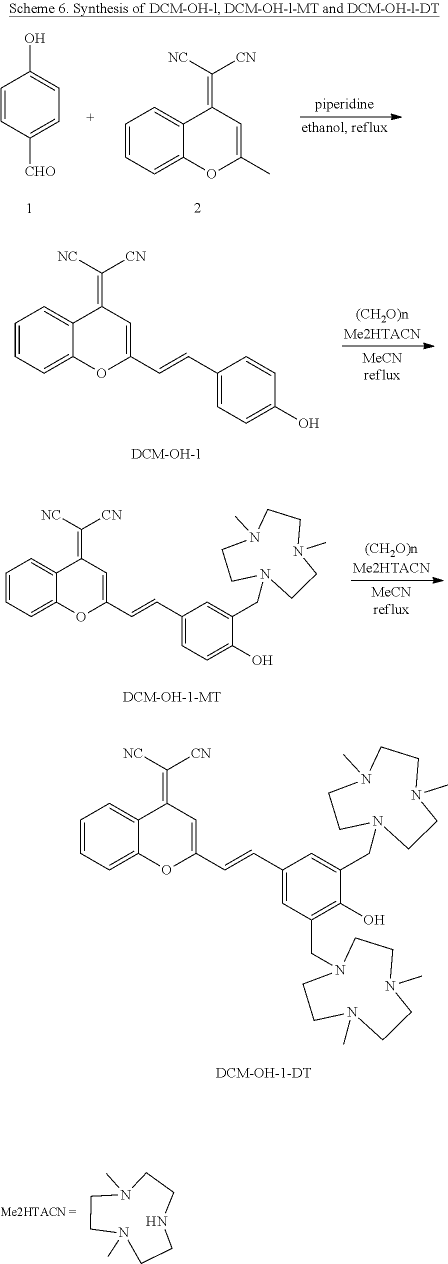 [SMILES: O=Cc1ccc(O)cc1 ; CC1=CC(=C(C#N)C#N)c2ccccc2O1 ; N#CC(C#N)=C1C=C(/C=C/c2ccc(O)cc2)Oc2ccccc21 ; CN1CCN(C)CCN(Cc2cc(/C=C/C3=CC(=C(C#N)C#N)c4ccccc4O3)ccc2O)CC1 ; CN1CCN(C)CCN(Cc2cc(/C=C/C3=CC(=C(C#N)C#N)c4ccccc4O3)cc(CN3CCN(C)CCN(C)CC3)c2O)CC1 ; CN1CCNCCN(C)CC1]

Compound 2 was obtained by following a reported procedure (Chemical Communications 2019, 55 (17), 2541-2544).

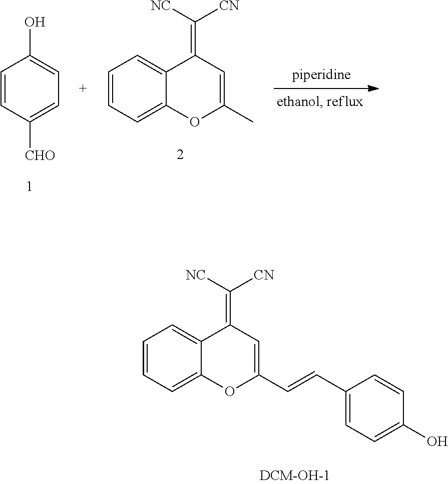 [SMILES: O=Cc1ccc(O)cc1 ; CC1=CC(=C(C#N)C#N)c2ccccc2O1 ; N#CC(C#N)=C1C=C(/C=C/c2ccc(O)cc2)Oc2ccccc21]

Synthesis of DCM-OH-1. To a solution of compound 1, 4-hydroxybenzaldehyde (364 mg, 2.98 mmol, 1 eq) and compound 2, 2-(2-methyl-4H-chromen-4-ylidene)malononitrile (620 mg, 2.98 mmol, 1 eq) in 20 mL ethanol, piperidine (1.02 g, 11.8 mmol, 4 eq) was added dropwise. The whole mixture was refluxed overnight. The reaction was then cooled down to room temperature and the solvent was removed under vacuum. The resulting residue was redissolved in ethyl acetate and washed with brine (containing 0.5 mL conc. HCl), the organic layer was collected and concentrated. The following recrystallization was done in MeCN and the compound DCM-OH-1 (372 mg, 1.19 mmol) was obtained as a dark red solid in 40% yield. ¹H NMR (500 MHz, Chloroform-d) δ 8.92 (d, J=8.4 Hz, 1H), 7.75 (t, J=7.7 Hz, 1H), 7.62-7.54 (m, 2H), 7.51 (d, J=8.4 Hz, 2H), 7.46 (t, J=7.7 Hz, 2H), 6.94 (d, J=8.4 Hz, 2H), 6.83 (s, 1H), 6.67 (d, J=15.9 Hz, 1H). HRMS-ESI (m/z): [M−H]⁻ calcd for C₂₀H₁₃N₂O₂, 311.0826; found 311.0816.

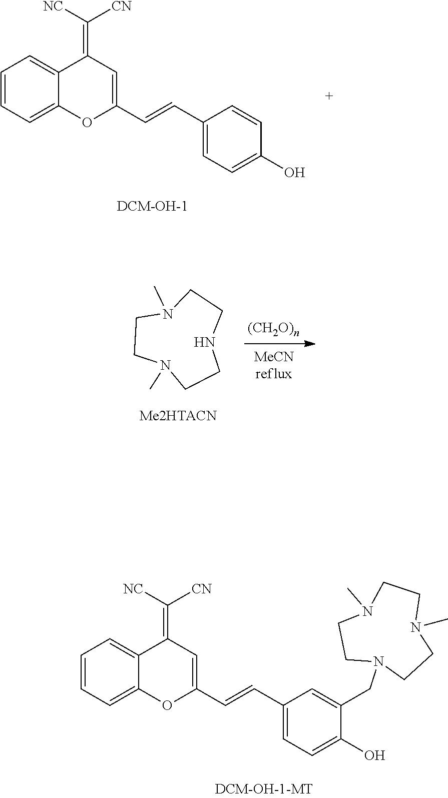 [SMILES: N#CC(C#N)=C1C=C(/C=C/c2ccc(O)cc2)Oc2ccccc21 ; CN1CCNCCN(C)CC1 ; CN1CCN(C)CCN(Cc2cc(/C=C/C3=CC(=C(C#N)C#N)c4ccccc4O3)ccc2O)CC1]

Synthesis of DCM-OH-1-MT. Paraformaldehyde (8 mg, 0.30 mmol) was added to a solution of 1,4-dimethyl-1,4,7-triazacyclononane Me2HTAC (32 mg, 0.20 mmol) in MeCN (20 mL), and the mixture was refluxed for half an hour. After which, DCM-OH-1 (64 mg, 0.20 mmol) was added to the reaction mixture. After refluxing for another 16 h, the reaction was cooled down to room temperature. The solvent was then removed under vacuum, and the resulting residue was purified by C-18 reversed-phase column (eluent: MeCN/H₂O with 0.1% TFA, gradient wash from 10:90 to 50:50). Compound DCM-OH-1-MT (41 mg 0.085 mmol) was obtained in 42% isolated yield. ¹H NMR (500 MHz, Chloroform-d) δ 8.84 (d, J=8.3 Hz, 1H), 7.65 (t, J=7.8 Hz, 1H), 7.54-7.42 (m, 2H), 7.41-7.28 (m, 2H), 7.20 (d, J=6.7 Hz, 3H), 6.86 (d, J=8.3 Hz, 1H), 6.73 (s, 1H), 6.55 (d, J=15.8 Hz, 1H), 3.81 (s, 2H), 2.89-2.79 (m, 4H), 2.66-2.59 (m, 4H), 2.57 (s, 4H), 2.37 (s, 6H). ¹³C NMR (126 MHz, CDCl₃) δ 160.85, 157.41, 151.91, 151.37, 138.46, 133.38, 128.82, 127.66, 124.80, 124.24, 123.12, 117.48, 116.94, 116.15, 115.08, 113.82, 104.77, 60.31, 59.00, 56.19, 55.88, 51.48, 44.88, 30.91, 28.68, 28.35, 21.67, 13.11. HRMS-ESI (m/z): [M+H]⁺ calcd for C₂₉H₃₂N₅O₂, 482.2550; found 482.2552.

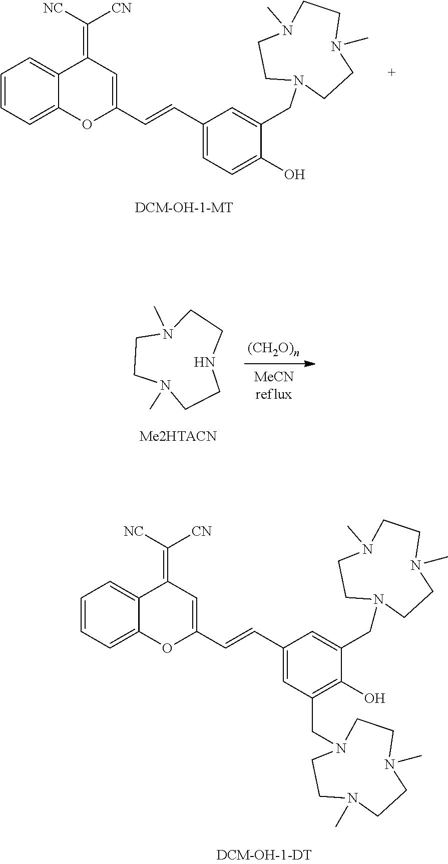 [SMILES: CN1CCN(C)CCN(Cc2cc(/C=C/C3=CC(=C(C#N)C#N)c4ccccc4O3)ccc2O)CC1 ; CN1CCNCCN(C)CC1 ; CN1CCN(C)CCN(Cc2cc(/C=C/C3=CC(=C(C#N)C#N)c4ccccc4O3)cc(CN3CCN(C)CCN(C)CC3)c2O)CC1]

Synthesis of DCM-OH-1-DT. Paraformaldehyde (8 mg, 0.30 mmol) was added to a solution of 1,4-dimethyl-1,4,7-triazacyclononane Me2HTAC (32 mg, 0.20 mmol) in MeCN (20 mL), and the mixture was refluxed for half an hour. Then DCM-OH-1-MT (30 mg, 0.06 mmol) was added to the reaction mixture. After refluxing for another 16 h, the reaction was cooled down to room temperature. The solvent was then removed under vacuum, and the resulting residue was purified by C-18 reversed-phase column (eluent: MeCN/H₂O with 0.1% TFA, gradient wash from 10:90 to 30:70). Compound DCM-OH-1-DT (41 mg 0.085 mmol) was obtained in 25% isolated yield. ¹H NMR (500 MHz, Chloroform-d) δ 8.87-8.83 (m, 2H), 7.66 (td, J=7.9, 7.3, 1.5 Hz, 2H), 7.48 (dd, J=12.1, 3.8 Hz, 2H), 7.38 (td, J=7.8, 7.3, 1.4 Hz, 1H), 7.30 (s, 2H), 6.76 (s, 1H), 6.59 (d, J=15.9 Hz, 1H), 3.91 (s, 4H), 2.91 (m, 12H), 2.79 (m, 12H), 2.48 (s, 12H). ¹³C NMR (126 MHz, Chloroform-d) δ 157.37, 151.89, 151.37, 138.40, 133.40, 129.18, 128.14, 124.79, 124.25, 123.42, 117.48, 116.93, 116.67, 116.12, 115.07, 113.89, 104.77, 60.36, 44.18, 30.91, 28.69, 28.35, 21.68, 13.11. HRMS-ESI (m/z): [M+H]⁺ calcd for C₃₈H₅₁N₈O₂, 651.4129; found 651.4110.

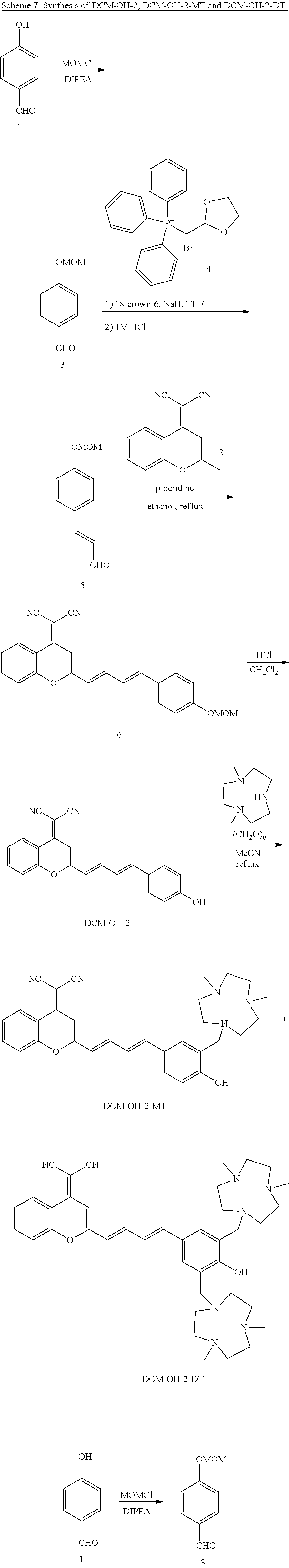

Synthesis of 4-(methoxymethoxy)benzaldehyde (3). To a solution of compound 1, 4-hydroxybenzaldehyde (500 mg, 4.09 mmol, 1 eq) in 30 mL CH₂Cl₂, N-ethyl-N-isopropylpropan-2-amine, and DIPEA (1.59 g, 12.3 mmol, 3 eq) were added dropwise, followed by the addition of chloro(methoxy)methane, MOMCl (989 mg, 12.3 mmol, 3 eq). The resulting reaction mixture was stirred at room temperature for 12 h and then poured into another 50 mL CH₂Cl₂. The combined solution was washed with brine and the organic layer was dried over MgSO₄ and concentrated. The residue was purified by flash column chromatography on silica gel (eluent: ethyl acetate/hexane from 0 to 30%). Compound 3 (490 mg, 2.95 mmol) was obtained in 75% isolated yield. ¹H NMR (500 MHz, Chloroform-d) δ 9.87 (s, 1H), 7.81 (d, J=8.7 Hz, 2H), 7.12 (d, J=8.6 Hz, 2H), 5.23 (s, 2H), 3.46 (s, 3H). ¹³C NMR (126 MHz, Chloroform-d) δ 191.11, 162.42, 132.08, 130.92, 116.48, 94.29, 56.57.

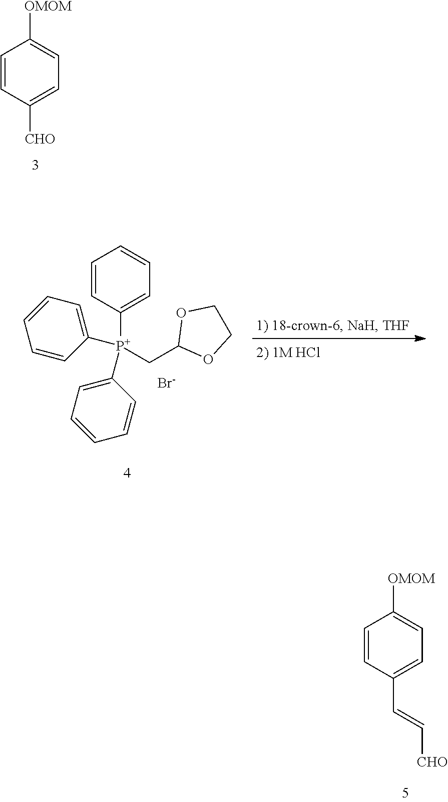

Synthesis of (E)-3-(4-(methoxymethoxy)phenyl)acrylaldehyde (5). Compound 3 (190 mg, 1.14 mmol, 1.0 eq), compound 4, ((1,3-dioxolan-2-yl)methyl)triphenylphosphonium bromide (690 mg, 1.6 mmol, 1.4 eq), and 18-crown-6 (3.7 mg, 0.11 mmol, 0.1 eq) were dissolved in 30 mL anhydrous THF. To the solution mixture, 60% NaH (212 mg, 5.1 mmol, 4.5 eq) in THF was added portion-wise. The whole mixture was stirred under nitrogen for 6 h. The reaction was then quenched by 1M HCl and neutralized with ammonia water. The volatile was removed under vacuum, and the residue was dilute with CH₂Cl₂ and then washed with brine. The organic layer was collected, dried over MgSO₄ and concentrated. The residue was purified by flash column chromatography on silica gel (eluent: ethyl acetate/hexane from 0 to 30%). Compound 5 (96 mg, 0.50 mmol) was obtained in 44% isolated yield. ¹H NMR (500 MHz, Chloroform-d) δ 9.61 (dd, J=7.8, 1.2 Hz, 1H), 7.47 (d, J=8.7 Hz, 1H), 7.38 (d, J=15.8 Hz, 1H), 7.05 (d, J=8.7 Hz, 1H), 6.57 (ddd, J=15.9, 7.7, 1.2 Hz, 1H), 5.18 (d, J=1.2 Hz, 2H), 3.44 (d, J=1.2 Hz, 3H). ¹³C NMR (126 MHz, Chloroform-d) δ 193.82, 159.99, 152.72, 130.49, 127.98, 127.09, 116.85, 94.35, 56.41.

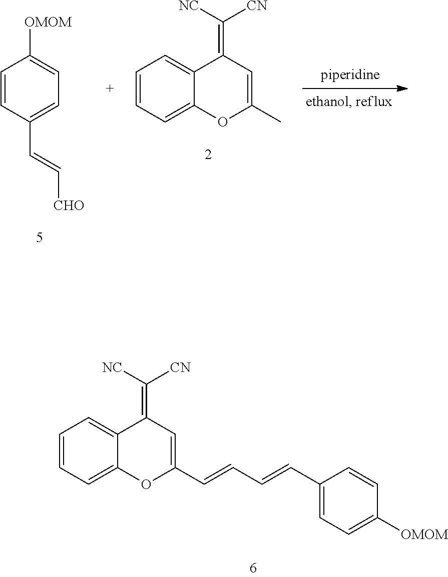

Synthesis of 2-(2-((1E, 3E)-4-(4-(methoxymethoxy)phenyl)buta-1, 3-dien-1-yl)-4H-chromen-4-ylidene)malononitrile (6). To a solution of compound 5 (58 mg, 0.3 mmol, 1 eq) and compound 2 (63 mg, 0.3 mmol, 1 eq) in 15 mL ethanol, piperidine (128 mg, 1.5 mmol, 5 eq) was added. The reaction mixture was refluxed for 12 h, after which it was cooled down to room temperature. The solid precipitate was collected and washed with cold methanol to generate compound 6 (42 mg, 0.11 mmol) in 37% yield. Compound 6 was used for the next step reaction without further purification. ¹H NMR (500 MHz, Methylene Chloride-d2) δ 8.79 (d, J=8.3 Hz, 1H), 7.66 (s, 1H), 7.47 (d, J=8.3 Hz, 1H), 7.44-7.32 (m, 4H), 6.96 (d, J=8.6 Hz, 2H), 6.92-6.80 (m, 2H), 6.70 (s, 1H), 6.30 (d, J=15.1 Hz, 1H), 5.12 (s, 2H), 3.38 (s, 3H).

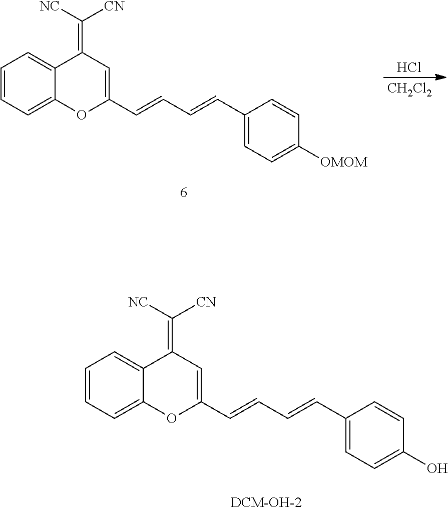

Synthesis of compound DCM-OH-2. Compound 6 (35 mg, 0.10 mmol) was dissolved in a mixture of 10 mL CH₂Cl₂ and 10 mL methanol, then 1 mL conc. HCl was added dropwise to the mixture. The reaction was stirred at room temperature overnight. The mixture was then diluted with CH₂Cl₂, and the organic solution was washed with a saturated sodium bicarbonate solution. The organic layer was dried over MgSO₄ and concentrated. The residue was purified by flash column chromatography on silica gel (eluent: ethyl acetate/hexane from 0 to 50%). Compound DCM-OH-2 (27 mg, 0.08 mmol) was obtained in 87% isolated yield. ¹H NMR (500 MHz, Acetone-d6) δ 8.85 (dd, J=8.3, 1.4 Hz, 1H), 7.94-7.85 (m, 1H), 7.77-7.71 (m, 1H), 7.67-7.54 (m, 2H), 7.54-7.43 (m, 2H), 7.10-7.01 (m, 2H), 6.92-6.87 (m, 2H), 6.84 (s, 1H), 6.71 (d, J=15.1 Hz, 1H). HRMS-ESI (m/z): [M]⁺ calcd for C₂₂H₁₅N₂O₂, 339.1127; found 339.1093.

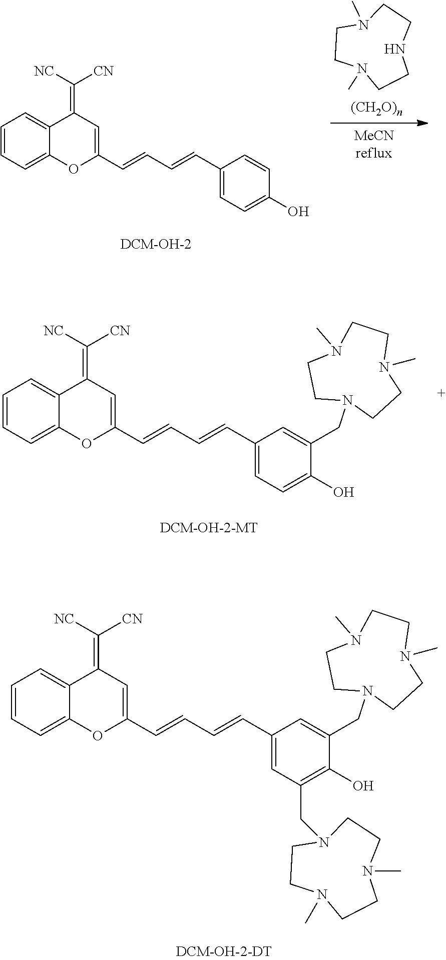 [SMILES: N#CC(C#N)=C1C=C(/C=C/C=C/c2ccc(O)cc2)Oc2ccccc21 ; CN1CCNCCN(C)CC1 ; CN1CCN(C)CCN(Cc2cc(/C=C/C=C/C3=CC(=C(C#N)C#N)c4ccccc4O3)ccc2O)CC1 ; CN1CCN(C)CCN(Cc2cc(/C=C/C=C/C3=CC(=C(C#N)C#N)c4ccccc4O3)cc(CN3CCN(C)CCN(C)CC3)c2O)CC1]

Synthesis of compound DCM-OH-2-MT and DCM-OH-2-DT. Paraformaldehyde (4.8 mg, 0.16 mmol) was added to a solution of 1,4-dimethyl-1,4,7-triazacyclononane Me2HTAC (12.5 mg, 0.08 mmol) in MeCN (20 mL), and the mixture was refluxed for half an hour. Then DCM-OH-2 (27 mg, 0.08 mmol) was added to the reaction mixture. After refluxing for another 24 h, the reaction was cooled down to room temperature. The solvent was then removed under vacuum, and the resulting residue was purified by C-18 reversed-phase column (eluent: MeCN/H₂O with 0.1% TFA, gradient wash from 10:90 to 30:70 to 50:50). Compound DCM-OH-2-MT (8 mg) and compound DCM-OH-2-DT (6 mg) was obtained in 20% and 11% isolated yield, respectively.

DCM-OH-2-DT, ¹H NMR (500 MHz, Acetone-d6) δ 8.86 (dd, J=8.3, 1.5 Hz, 1H), 7.91 (ddd, J=8.6, 7.2, 1.5 Hz, 1H), 7.73 (dd, J=8.5, 1.4 Hz, 1H), 7.69-7.55 (m, 2H), 7.49 (s, 2H), 7.09 (m, 2H), 6.83 (s, 2H), 6.69 (d, J=15.0 Hz, 1H), 4.05 (s, 4H), 3.04 (m, 8H), 2.90 (m, 16H), 2.56 (s, 12H). ¹³C NMR (126 MHz, Acetone-d6) δ 158.68, 152.65, 152.51, 141.32, 140.78, 134.96, 129.70, 125.86, 125.12, 118.84, 117.59, 117.04, 115.66, 105.60, 57.70, 54.58, 51.40, 44.04, 31.74, 24.87, 22.44, 13.46. HRMS-ESI (m/z): [M+H]⁺ calcd for C₄₀H₅₃N₈O₂, 677.4285; found 677.4250.

DCM-OH-2-MT, ¹H NMR (500 MHz, Acetone-d6) δ 8.86 (dd, J=8.4, 1.4 Hz, 1H), 7.91 (ddd, J=8.5, 7.2, 1.5 Hz, 1H), 7.74 (dd, J=8.4, 1.3 Hz, 1H), 7.68-7.55 (m, 2H), 7.42 (dd, J=8.4, 2.2 Hz, 1H), 7.39 (d, J=2.2 Hz, 1H), 7.09-7.03 (m, 2H), 6.96 (d, J=8.3 Hz, 1H), 6.85 (s, 1H), 6.70 (d, J=15.0 Hz, 1H), 3.92 (s, 2H), 2.97-2.88 (m, 4H), 2.75 (dd, J=9.3, 3.8 Hz, 8H), 2.49 (s, 6H). ¹³C NMR (126 MHz, Acetone-d6) δ 159.04, 153.08, 152.90, 135.32, 129.46, 129.03, 127.38, 126.22, 125.50, 125.08, 124.53, 120.59, 119.23, 117.89, 117.14, 115.99, 106.02, 61.02, 32.13, 22.82, 13.84. HRMS-ESI (m/z): [M+H]⁺ calcd for C₃₁H₃₄N₅O₂, 508.2713; found 508.2715.

### Example 3. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’, which also includes complexes of the compound):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 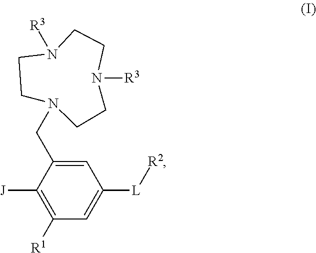
or
a salt, metal complex, or combination thereof, wherein optionally a metal of the metal complex is copper, a copper ion, or a radioisotope thereof;
wherein
J is OH, SH, or N(R³)₂;
L is —(CH═CH)ₙ— wherein n is 2, 1, or 0 wherein L is a direct bond between its two points of attachment when n is 0;
R¹ is —CH₂N(CH₂CH₂NR³CH₂)₂, H, or —OR³;
R² is: 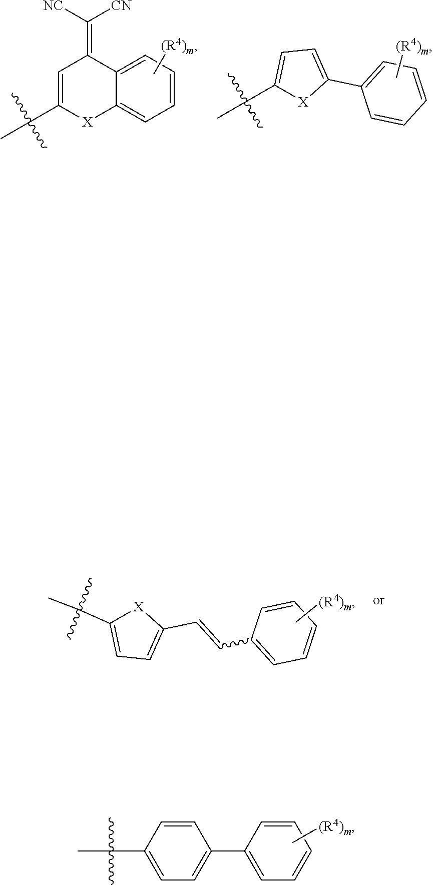
wherein
R⁴ is —C(═O)R³, —N(R³)₂, —OR³, or halo;
X is O, S, or NR³; and
m is 0, 1, 2, or 3; or
R² is a substituted phenyl; and
each R³ is independently —(C₁-C₆)alkyl or H;

2. The compound of claim 1 wherein J is OH.

3. The compound of claim 1 wherein L is —(CH═CH)₂— or —CH═CH—, wherein optionally L has the E-configuration.

4. The compound of claim 1 wherein R¹ is —CH₂N(CH₂CH₂NR³CH₂)₂.

5. The compound of claim 1 wherein R² is:

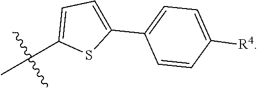

6. The compound of claim 5 wherein R⁴ is —C(═O)H or —N(CH₃)₂.

7. The compound of claim 1 wherein L is the direct bond and R² is: 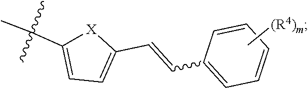
wherein
R⁴ is —C(═O)R³, —N(R³)₂, —OR³, or halo;
X is O, S, or NR³; and
m is 1, 2, or 3.

8. The compound of claim 7 wherein R² is

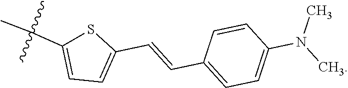 [SMILES: *c1ccc(/C=C/c2ccc(N(C)C)cc2)s1]

9. The compound of claim 1 wherein R² is:

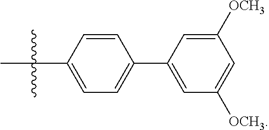 [SMILES: *c1ccc(-c2cc(OC)cc(OC)c2)cc1]

10. The compound of claim 1 represented by Formula II: 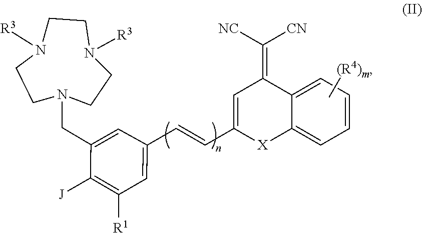
or a salt, metal complex, or combination thereof;
wherein n is 2 or 1.

11. The compound of claim 1 represented by Formula III, IV, or V: 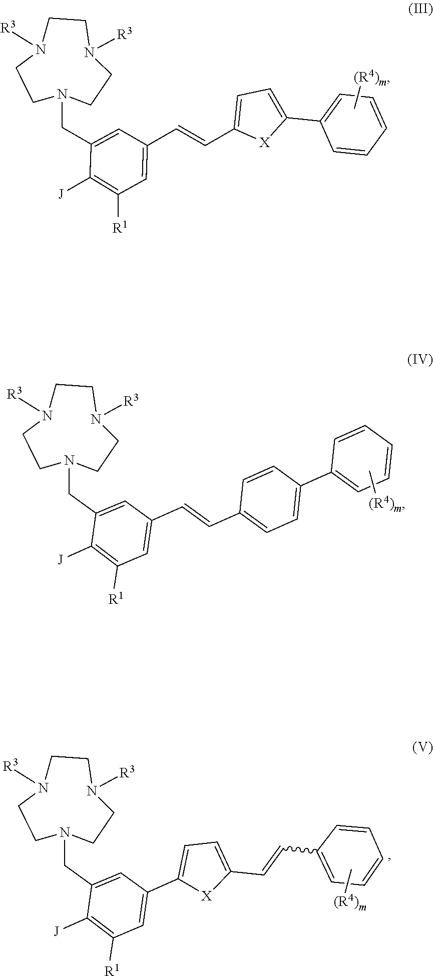
or
a salt, metal complex, or combination thereof.

12. The compound of claim 1 wherein the compound is:
2-(2-((1E,3E)-4-(3,5-bis((4,7-dimethyl-1,4,7-triazonan-1-yl)methyl)-4-hydroxyphenyl)buta-1,3-dien-1-yl)-4H-chromen-4-ylidene)malononitrile (DCM-OH-2-DT);
(E)-2-(2-(3-((4,7-dimethyl-1,4,7-triazonan-1-yl)methyl)-4-hydroxystyryl)-4H-chromen-4-ylidene)malononitrile (DCM-OH-1-MT);
2-(2-((1E,3E)-4-(3-((4,7-dimethyl-1,4,7-triazonan-1-yl)methyl)-4-hydroxyphenyl)buta-1,3-dien-1-yl)-4H-chromen-4-ylidene)malononitrile (DCM-OH-2-MT);
(E)-2-(2-(3, 5-bis((4,7-dimethyl-1,4,7-triazonan-1-yl)methyl)-4-hydroxystyryl)-4H-chromen-4-ylidene)malononitrile (DCM-OH-1-DT);
or a salt, metal complex, or combination thereof; or
wherein the compound is:
(E)-2-((4,7-dimethyl-1,4,7-triazonan-1-yl)methyl)-4-(2-(5-(4-(dimethylamino)phenyl)thiophen-2-yl)vinyl)phenol (ZY-5-MT);
(E)-2-((4,7-dimethyl-1,4,7-triazonan-1-yl)methyl)-4-(2-(5-(4-(dimethylamino)phenyl)thiophen-2-yl)vinyl)-6-methoxyphenol (ZY-5-OMe);
(E)-2,6-bis((4,7-dimethyl-1,4,7-triazonan-1-yl)methyl)-4-(2-(5-(4-(dimethylamino)phenyl)thiophen-2-yl)vinyl)phenol (ZY-5-DT);
(E)-4-(5-(3-((4,7-dimethyl-1,4,7-triazonan-1-yl)methyl)-4-hydroxystyryl)thiophen-2-yl)benzaldehyde (ZY-15-MT);
(E)-4-(5-(3-((4,7-dimethyl-1,4,7-triazonan-1-yl)methyl)-4-hydroxy-5-methoxystyryl)thiophen-2-yl)benzaldehyde (ZY-15-OMe);
(E)-4-(5-(3,5-bis((4,7-dimethyl-1,4,7-triazonan-1-yl)methyl)-4-hydroxystyryl)thiophen-2-yl)benzaldehyde (ZY-15-DT);
or a salt, metal complex, or combination thereof; or
wherein the compound is:
(E)-4-(2-(3′,5′-dimethoxy-[1,1′-biphenyl]-4-yl)vinyl)-2-((4,7-dimethyl-1,4,7-triazonan-1-yl)methyl)phenol (ZY-17-MT);
(E)-4-(2-(3′,5′-dimethoxy-[1,1′-biphenyl]-4-yl)vinyl)-2-((4,7-dimethyl-1,4,7-triazonan-1-yl)methyl)-6-methoxyphenol (ZY-17-OMe);
(E)-4-(2-(3′,5′-dimethoxy-[1,1′-biphenyl]-4-yl)vinyl)-2,6-bis((4,7-dimethyl-1,4,7-triazonan-1-yl)methyl)phenol (ZY-17-DT);
or a salt, metal complex, or combination thereof; or
wherein the compound is:
(E)-2-((4,7-dimethyl-1,4,7-triazonan-1-yl)methyl)-4-(5-(4-(dimethylamino)styryl)thiophen-2-yl)phenol (ZY-12-MT);
(E)-2-((4,7-dimethyl-1,4,7-triazonan-1-yl)methyl)-4-(5-(4-(dimethylamino)styryl)thiophen-2-yl)-6-methoxyphenol (ZY-12-OMe);
(E)-2,6-bis((4,7-dimethyl-1,4,7-triazonan-1-yl)methyl)-4-(5-(4-(dimethylamino)styryl)thiophen-2-yl)phenol (ZY-12-DT);
or a salt, metal complex, or combination thereof.

13. A method for positron emission tomography (PET) imaging amyloid-beta oligomers comprising:
a) administering to a subject a composition comprising a ⁶⁴Cu metal complex of the compound according to claim 1;
b) imaging the brain of the subject for the presence or absence of amyloid-beta oligomers by PET;
wherein the ⁶⁴Cu metal complex selectively binds to the amyloid-beta oligomers when present, thereby providing an early diagnosis for Alzheimer's disease.

14. A method for near infrared (NIR) imaging amyloid-beta oligomers comprising:
a) administering to a subject a composition comprising a compound according to claim 1;
b) imaging the brain of the subject for the presence or absence of amyloid-beta oligomers by NIR;
wherein the compound selectively binds to the amyloid-beta oligomers when present, thereby providing an early diagnosis for Alzheimer's disease.

15. A method for reducing the neurotoxicity of amyloid-beta oligomers comprising administering to a subject suffering from an early onset of Alzheimer's disease an effective amount of a composition comprising a compound according to claim 1, wherein the compound selectively binds to amyloid-beta oligomers present in the brain of the subject and reduces the neurotoxicity of amyloid-beta oligomers.

16. The method of claim 15 wherein the amyloid-beta oligomers are amyloid-beta-42 oligomers;

17. The method of claim 15 wherein the amyloid-beta oligomers comprise Cu²⁺.
